SEQUENCE LISTING (44 sequences)

SEQ ID NO: 1 - DNA, 3992 bp, Homo sapiens
ataaaagctgcccggggaagccaggagagcgaagggcggacgtactcgccacggcacccaggctgcgcgcacgcggtcccggtgtgcagctggagagcgagcggccaccgggagcccccggcacagcccgcgcccgccccgcaggagcccgcgaagatgccccggcgcagcctgcacgcggcggccgtgctcctgctggtgatcttaaaggaacagccttccagcccggccccagtgaacggttccaagtggacttattttggtcctgatggggagaatagctggtccaagaagtacccgtcgtgtgggggcctgctgcagtcccccatagacctgcacagtgacatcctccagtatgacgccagcctcacgcccctcgagttccaaggctacaatctgtctgccaacaagcagtttctcctgaccaacaatggccattcagtgaagctgaacctgccctcggacatgcacatccagggcctccagtctcgctacagtgccacgcagctgcacctgcactgggggaacccgaatgacccgcacggctctgagcacaccgtcagcggacagcacttcgccgccgagctgcacattgtccattataactcagacctttatcctgacgccagcactgccagcaacaagtcagaaggcctcgctgtcctggctgttctcattgagatgggctccttcaatccgtcctatgacaagatcttcagtcaccttcaacatgtaaagtacaaaggccaggaagcattcgtcccgggattcaacattgaagagctgcttccggagaggaccgctgaatattaccgctaccgggggtccctgaccacacccccttgcaaccccactgtgctctggacagttttccgaaaccccgtgcaaatttcccaggagcagctgctggctttggagacagccctgtactgcacacacatggacgacccttcccccagagaaatgatcaacaacttccggcaggtccagaagttcgatgagaggctggtatacacctccttctcccaagtgcaagtctgtactgcggcaggactgagtctgggcatcatcctctcactggccctggctggcattcttggcatctgtattgtggtggtggtgtccatttggcttttcagaaggaagagtatcaaaaaaggtgataacaagggagtcatttacaagccagccaccaagatggagactgaggcccacgcttgaggtccccggagctcccgggcacatccaggaaggaccttgctttggaccctacacacttcggctctctggacacttgcgacacctcaaggtgttctctgtagctcaatctgcaaacatgccaggcctcagggatcctctgctgggtgcctccttgccttgggaccatggccaccccagagccatccgatcgatggatgggatgcactctcagaccaagcagcaggaattcaaagctgcttgctgtaactgtgtgagattgtgaagtggtctgaattctggaatcacaaaccaagccatgctggtgggccattaatggttggaaaacactttcatccggggctttgccagagcgtgctttcaagtgtcctggaaattctgctgcttctccaagctttcagacaagaatgtgcactctctgcttaggttttgcttgggaaactcaacttctttcctctggagacggggcatctccctctgatttccttctgctatgacaaaacctttaatctgcaccttacaactcggggacaaatggggacaggaaggatcaagttgtagagagaaaaagaaaacaagagatatacattgtgatatattagggacactttcacagtcctgtcctctggatcacagacactgcacagaccttagggaatggcaggttcaagttccacttcttggtggggatgagaagggagagagagctagagggacaaagagaatgagaagacatggatgatctgggagagtctcactttggaatcagaattggaatcacattctgtttatcaagccataatgtaaggacagaataatacaatattaagtccaaatccaacctcctgtcagtggagcagttatgttttatactctacagattttacaaataatgaggctgttccttgaaaatgtgttgttgctgtgtcctggaggagacatgagttccgagatgacccaatctgcctttgaatctggaggaaataggcagaaacaaaatgactgtagaacttattctctgtaggccaaatttcatttcagccacttctgcaggatccctactgccaacctggaatggagacttttatctacttctctctctctgaagatgtcaaatcgtggtttagatcaaatatatttcaagctataaaagcaggaggttatctgtgcagggggctggcatcatgtatttaggggcaagtaataatggaatgctactaagatactccatattcttccccgaatcacacagacagtttctgacaggcgcaactcctccattttcctcccgcaggtgagaaccctgtggagatgagtcagtgccatgactgagaaggaaccgacccctagttgagagcaccttgcagttccccgagaactttctgattcacagtctcattttgacagcatgaaatgtcctcttgaagcatagctttttaaatatctttttccttctactcctccctctgactctaagaattctctcttctggaatcgcttgaacccaggaggcggaggttgcagtaagccaaggtcatgccactgcactctagcctgggtgacagagcgagactccatctcaaaaaaaaaaaaaaaaaaattattctgtaccatcacaacttttcacaacgatggcaagccttatgtcttgggagcctgttttgctaggcaaagttacaagtgacctaatgggagctcaaatgtgtgtgtgtctctctgtgtgtttgtgtgtgtgtgtgcactcaagacctctaacagcctcgaagcctggggtggcatcccggccttgccattagcatgcctcatgcatcatcagatgacaaggacaaccctcatgacgaagcaacatgaattagggggcctcttggccttggtccaaaattgtcaatcagaaatgaacataaaggactccagagcagtgggactgtctgtcaaaagactctgtatatcttttgtggatgagttttgtgagagaacagagagaccattgtacctggcacaagggctgttcatgaaaagggagacttactgggaggtgcaagacagtggcatttctcctctcctcttgctgctcagcacagccctggattgcagccccgaggctgagaccagacaaagcccgggaggcagaaagatgctccaagaaccaacactatcaatgtctttgcaaatcctcacaggattcctgtgggtccagctttggaactgggaaacctttcttcggatccgcactcattccactgatgccagctgcccctgaaggatgccagtactgtggtgtgtgagtctcagcagccgcccacacgctcctaactctgctgcatggcagatgcctaggtggaaatagcaaaaacaaggcccaggctggggccagggccagaggggaaggccctggattctcactcatgtgagatcttgaatctctttctttgttctgtttgtttagttagtatcatctggtaaaatagttaaaaaacaacaaaaaactctgtatctgtttctagcatgtgctgcattgactctattaatcacatttcaaattcaccctacattcctctcctcttcactagcctctctgaaggtgtcctggccagccctggagaagcactggtgtctgcagcacccctcagttcctgtgcctcagcccacaggccactgtgataatggtctgtttagcacttctgtatttattgtaagaatgattataatgaagatacacactgtaactacaagaaattataaatgtttttcacatcaaaaaaaaaaaaaaaaaa

SEQ ID NO: 2 - DNA, 1561 bp, Homo sapiens
gcccgtacacaccgtgtgctgggacaccccacagtcagccgcatggctcccctgtgccccagcccctggctccctctgttgatcccggcccctgctccaggcctcactgtgcaactgctgctgtcactgctgcttctggtgcctgtccatccccagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaaggggatgaccagagtcattggcgctatggaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtgtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgagatccacgtggttcacctcagcaccgcctttgccagagttgacgaggccttggggcgcccgggaggcctggccgtgttggccgcctttctggaggagggcccggaagaaaacagtgcctatgagcagttgctgtctcgcttggaagaaatcgctgaggaaggctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcagctccacaccctctctgacaccctgtggggacctggtgactctcggctacagctgaacttccgagcgacgcagcctttgaatgggcgagtgattgaggcctccttccctgctggagtggacagcagtcctcgggctgctgagccagtccagctgaattcctgcctggctgctggtgacatcctagccctggtttttggcctcctttttgctgtcaccagcgtcgcgttccttgtgcagatgagaaggcagcacagaaggggaaccaaagggggtgtgagctaccgcccagcagaggtagccgagactggagcctagaggctggatcttggagaatgtgagaagccagccagaggcatctgagggggagccggtaactgtcctgtcctgctcattatgccacttccttttaactgccaagaaattttttaaaataaatatttataataaaaaaaaa

SEQ ID NO: 3 - DNA, 2722 bp, Homo sapiens
gagtctggccgcagtcgcggcagtggtggcttcccatccccaaaaggcgccctccgactccttgcgccgcactgctcgccgggccagtccggaaacgggtcgtggagctccgcaccactcccgctggttcccgaaggcagatcccttctcccgagagttgcgagaaactttcccttgtccccgacgctgcagcggctcgggtaccgtggcagccgcaggtttctgaaccccgggccacgctccccgcgcctcggcttcgcgctcgtgtagatcgttccctctctggttgcacgctggggatcccggacctcgattctgcgggcgagatgcccctgggacacatcatgaggctggacctggagaaaattgccctggagtacatcgtgccctgtctgcacgaggtgggcttctgctacctggacaacttcctgggcgaggtggtgggcgactgcgtcctggagcgcgtcaagcagctgcactgcaccggggccctgcgggacggccagctggcggggccgcgcgccggcgtctccaagcgacacctgcggggcgaccagatcacgtggatcgggggcaacgaggagggctgcgaggccatcagcttcctcctgtccctcatcgacaggctggtcctctactgcgggagccggctgggcaaatactacgtcaaggagaggtctaaggcaatggtggcttgctatccgggaaatggaacaggttatgttcgccacgtggacaaccccaacggtgatggtcgctgcatcacctgcatctactatctgaacaagaattgggatgccaagctacatggtgggatcctgcggatatttccagaggggaaatcattcatagcagatgtggagcccatttttgacagactcctgttcttctggtcagatcgtaggaacccacacgaagtgcagccctcttacgcaaccagatatgctatgactgtctggtactttgatgctgaagaaagggcagaagccaaaaagaaattcaggaatttaactaggaaaactgaatctgccctcactgaagactgaccgtgctctgaaatctgctggccttgttcattttagtaacggttcctgaattctcttaaattctttgagatccaaagatggcctcttcagtgacaacaatctccctgctacttcttgcatccttcacatccctgtcttgtgtgtggtacttcatgttttcttgccaagactgtgttgatcttcagatactctctttgccagatgaagttacttgctaactccagaaattcctgcagacatcctactcggccagcggtttacctgatagattcggtaatactatcaagagaagagcctaggagcacagcgagggaatgaaccttacttgcactttatgtatacttcctgatttgaaaggaggaggtttgaaaagaaaaaaatggaggtggtagatgccacagagaggcatcacggaagccttaacagcaggaaacagagaaatttgtgtcatctgaacaatttccagatgttcttaatccagggctgttggggtttctggagaattatcacaacctaatgacattaatacctctagaaagggctgctgtcatagtgaacaatttataagtgtcccatggggcagacactccttttttcccagtcctgcaacctggattttctgcctcagccccattttgctgaaaataatgactttctgaataaagatggcaacacaattttttctccattttcagttcttacctgggaacctaattccccagaagctaaaaaactagacattagttgttttggttgctttgttggaatggaatttaaatttaaatgaaaggaaaaatatatccctggtagttttgtgttaaccactgataactgtggaaagagctaggtctactgatatacaataaacatgtgtgcatcttgaacaatttgagaggggaggtggagttggaaatgtgggtgttcctgttttttttttttttttttttttagttttcctttttaatgagctcaccctttaacacaaaaaaagcaaggtgatgtattttaaaaaaggaagtggaaataaaaaaatctcaaagctatttgagttctcgtctgtccctagcagtctttcttcagctcacttggctctctagatccactgtggttggcagtatgaccagaatcatggaatttgctagaactgtggaagcttctactcctgcagtaagcacagatcgcactgcctcaataacttggtattgagcacgtattttgcaaaagctacttttcctagttttcagtattactttcatgttttaaaaatccctttaatttcttgcttgaaaatcccatgaacattaaagagccagaaatattttcctttgttatgtacggatatatatatatatagtcttccaagatagaagtttactttttcctcttctggttttggaaaatttccagataagacatgtcaccattaattctcaacgactgctctattttgttgtacggtaatagttatcaccttctaaattactatgtaatttattcacttattatgtttattgtcttgtatcctttctctggagtgtaagcacaatgaagacaggaattttgtatatttttaaccaatgcaacatactctcagcacctaaaatagtgccgggaacatagtaagggctcagtaaatacttgttgaataaactcagtctcctacattagcattctaa

SEQ ID NO: 4 - DNA, 1432 bp, Homo sapiens
ggggcgtgctcgcggctataaggggcggaggctgggcggcgttgctctgcgctctgcggctgacggcgcttttgtctccggtgagttttgtggcgggaagcttctgcgctggtgcttagtaaccgactttcctccggactcctgcacgacctgctcctacagccggcgatccactcccggctgttcccccggagggtccagaggcctttcagaaggagaaggcagctctgtttctctgcagaggagtagggtcctttcagccatgaagcatgtgttgaacctctacctgttaggtgtggtactgaccctactctccatcttcgttagagtgatggagtccctagagggcttactagagagcccatcgcctgggacctcctggaccaccagaagccaactagccaacacagagcccaccaagggccttccagaccatccatccagaagcatgtgataagacctccttccatactggccatattttggaacactgacctagacatgtccagatgggagtcccattcctagcagacaagctgagcaccgttgtaaccagagaactattactaggccttgaagaacctgtctaactggatgctcattgcctgggcaaggcctgtttaggccggttgcggtggctcatgcctgtaatcctagcactttgggaggctgaggtgggtggatcacctgaggtcaggagttcgagaccagcctcgccaacatggcgaaaccccatctctactaaaaatacaaaagttagctgggtgtggtggcagaggcctgtaatcccagctccttgggaggctgaggcgggagaattgcttgaacccggggacggaggttgcagtgagccgagatcgcactgctgtacccagcctgggccacagtgcaagactccatctcaaaaaaaaaagaaaagaaaaagcctgtttaatgcacaggtgtgagtggattgcttatggctatgagataggttgatctcgcccttaccccggggtctggtgtatgctgtgctttcctcagcagtatggctctgacatctcttagatgtcccaacttcagctgttgggagatggtgatattttcaaccctacttcctaaacatctgtctggggttcctttagtcttgaatgtcttatgctcaattatttggtgttgagcctctcttccacaagagctcctccatgtttggatagcagttgaagaggttgtgtgggtgggctgttgggagtgaggatggagtgttcagtgcccatttctcattttacattttaaagtcgttcctccaacatagtgtgtattggtctgaagggggtggtgggatgccaaagcctgctcaagttatggacattgtggccaccatgtggcttaaatgattttttctaactaataaagtggaatatatatttctaaaaaaaaaaaaaaaaaaaaa

SEQ ID NO: 5 - DNA, 3183 bp, Homo sapiens
gacagaagcaatggccgaggcagaagacaagccgaggtgctggtgaccctgggcgtctgagtggatgattggggctgctgcgctcagaggcctgcctccctgccttccaatgcatataaccccacaccccagccaatgaagacgagaggcagcgtgaacaaagtcatttagaaagcccccgaggaagtgtaaacaaaagagaaagcatgaatggagtgcctgagagacaagtgtgtcctgtactgcccccacctttagctgggccagcaactgcccggccctgcttctccccacctactcactggtgatctttttttttttacttttttttcccttttcttttccattctcttttcttattttctttcaaggcaaggcaaggattttgattttgggacccagccatggtccttctgcttcttctttaaaatacccactttctccccatcgccaagcggcgtttggcaatatcagatatccactctatttatttttacctaaggaaaaactccagctcccttcccactcccagctgccttgccacccctcccagccctctgcttgccctccacctggcctgctgggagtcagagcccagcaaaacctgtttagacacatggacaagaatcccagcgctacaaggcacacagtccgcttcttcgtcctcagggttgccagcgcttcctggaagtcctgaagctctcgcagtgcagtgagttcatgcaccttcttgccaagcctcagtctttgggatctggggaggccgcctggttttcctccctccttctgcacgtctgctggggtctcttcctctccaggccttgccgtccccctggcctctcttcccagctcacacatgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccgcaacttggaggagaactgctgtgtgcgccccctctacattgacttccgacaggatctgggctggaagtgggtccatgaacctaagggctactatgccaacttctgctcaggcccttgcccatacctccgcagtgcagacacaacccacagcacggtgctgggactgtacaacactctgaaccctgaagcatctgcctcgccttgctgcgtgccccaggacctggagcccctgaccatcctgtactatgttgggaggacccccaaagtggagcagctctccaacatggtggtgaagtcttgtaaatgtagctgagaccccacgtgcgacagagagaggggagagagaaccaccactgcctgactgcccgctcctcgggaaacacacaagcaacaaacctcactgagaggcctggagcccacaaccttcggctccgggcaaatggctgagatggaggtttccttttggaacatttctttcttgctggctctgagaatcacggtggtaaagaaagtgtgggtttggttagaggaaggctgaactcttcagaacacacagactttctgtgacgcagacagaggggatggggatagaggaaagggatggtaagttgagatgttgtgtggcaatgggatttgggctaccctaaagggagaaggaagggcagagaatggctgggtcagggccagactggaagacacttcagatctgaggttggatttgctcattgctgtaccacatctgctctagggaatctggattatgttatacaaggcaagcattttttttttttttttaaagacaggttacgaagacaaagtcccagaattgtatctcatactgtctgggattaagggcaaatctattacttttgcaaactgtcctctacatcaattaacatcgtgggtcactacagggagaaaatccaggtcatgcagttcctggcccatcaactgtattgggccttttggatatgctgaacgcagaagaaagggtggaaatcaaccctctcctgtctgccctctgggtccctcctctcacctctccctcgatcatatttccccttggacacttggttagacgccttccaggtcaggatgcacatttctggattgtggttccatgcagccttggggcattatgggttcttcccccacttcccctccaagaccctgtgttcatttggtgttcctggaagcaggtgctacaacatgtgaggcattcggggaagctgcacatgtgccacacagtgacttggccccagacgcatagactgaggtataaagacaagtatgaatattactctcaaaatctttgtataaataaatatttttggggcatcctggatgatttcatcttctggaatattgtttctagaacagtaaaagccttattctaaggtg

SEQ ID NO: 6 - DNA, 817 bp, Homo sapiens
agtcctgcgtccgggccccgaggcgcagcagggcaccaggtggagcaccagctacgcgtggcgcagcgcagcgtccctagcaccgagcctcccgcagccgccgagatgctgcgaacagagagctgccgccccaggtcgcccgccggacaggtggccgcggcgtccccgctcctgctgctgctgctgctgctcgcctggtgcgcgggcgcctgccgaggtgctccaatattacctcaaggattacagcctgaacaacagctacagttgtggaatgagatagatgatacttgttcgtcttttctgtccattgattctcagcctcaggcatccaacgcactggaggagctttgctttatgattatgggaatgctaccaaagcctcaggaacaagatgaaaaagataatactaaaaggttcttatttcattattcgaagacacagaagttgggcaagtcaaatgttgtgtcgtcagttgtgcatccgttgctgcagctcgttcctcacctgcatgagagaagaatgaagagattcagagtggacgaagaattccaaagtccctttgcaagtcaaagtcgaggatattttttattcaggccacggaatggaagaaggtcagcagggttcatttaaaatggatgccagctaattttccacagagcaatgctatggaatacaaaatgtactgacattttgttttcttctgaaaaaaatccttgctaaatgtactctgttgaaaatccctgtgttgtcaatgttctcagttgtaacaatgttgtaaatgttcaatttgttgaaaattaaaaaatctaaaaataaa

SEQ ID NO: 7 - DNA, 1828 bp, Homo sapiens
cagttacagggagcaccaccagggaacatctcggggagcctggttggaagctgcaggcttagtctgtcggctgcgggtctctgactgccctgtggggagggtcttgccttaacatcccttgcatttggctgcaaagaaatctgcttggaagaaggggttacgctgtttggccgggcagaaactccgctgagcagaacttgccgccagaatgctcctcctgttgctgagtatcatcgtcctccacgtcgcggtgctggtgctgctgttcgtctccacgatcgtcagccaatggatcgtgggcaatggacacgcaactgatctctggcagaactgtagcacctcttcctcaggaaatgtccaccactgtttctcatcatcaccaaacgaatggctgcagtctgtccaggccaccatgatcctgtcgatcatcttcagcattctgtctctgttcctgttcttctgccaactcttcaccctcaccaaggggggcaggttttacatcactggaatcttccaaattcttgctggtctgtgcgtgatgagtgctgcggccatctacacggtgaggcacccggagtggcatctcaactcggattactcctacggtttcgcctacatcctggcctgggtggccttccccctggcccttctcagcggtgtcatctatgtgatcttgcggaaacgcgaatgaggcgcccagacggtctgtctgaggctctgagcgtacatagggaagggaggaagggaaaacagaaagcagacaaagaaaaaagagctagcccaaaatcccaaactcaaaccaaaccaaacagaaagcagtggaggtgggggttgctgttgattgaagatgtatataatatctccggtttataaaacctatttataacactttttacatatatgtacatagtattgtttgctttttatgttgaccatcagcctcgtgttgagccttaaagaagtagctaaggaactttacatcctaacagtataatccagctcagtatttttgttttgttttttgtttgtttgttttgttttacccagaaataagataactccatctcgccccttccctttcatctgaaagaagatacctccctcccagtccacctcatttagaaaaccaaagtgtgggtagaaaccccaaatgtccaaaagcccttttctggtgggtgacccagtgcatccaacagaaacagccgctgcccgaacctctgtgtgaagctttacgcgcacacggacaaaatgcccaaactggagcccttgcaaaaacacggcttgtggcattggcatacttgcccttacaggtggagtatcttcgtcacacatctaaatgagaaatcagtgacaacaagtctttgaaatggtgctatggatttaccattccttattatcactaatcatctaaacaactcactggaaatccaattaacaattttacaacataagatagaatggagacctgaataattctgtgtaatataaatggtttataactgcttttgtacctagctaggctgctattattactataatgagtaaatcataaagccttcatcactcccacatttttcttacggtcggagcatcagaacaagcgtctagactccttgggaccgtgagttcctagagcttggctgggtctaggctgttctgtgcctccaaggactgtctggcaatgacttgtattggccaccaactgtagatgtatatatggtgcccttctgatgctaagactccagaccttttgtttttgctttgcattttctgattttataccaactgtgtggactaagatgcattaaaataaacatcagagtaactc

SEQ ID NO: 8 - DNA, 4846 bp, Homo sapiens
gagcggtgctcaggggagggctggaggggagggaaggagagagagaggggagggcggcaccgcccctagccccgcgctccggaagtgaagcggccagaccaccagctaatggatgcggagcggagggcccgctgaccgctctccgcgcctggagcagcttggcttggctggagctaagagccagacacaccactgtgtggaggtgggtgatgtcttcctgtgctaaaaggtgaataaataagctcctcacctctcgcggaacactcgggaacacatcaacaggggtccaagccgccctgctgggaggcttctcttcaagagttctgggtcccagagtggaaggcattttcccatcaactggagagagacgaaacatcagagaccaggaggctgtggagaaagcagctgtcccaggtgcctcaactatcagagaagggtcagcgtcacgtggctgccagcatctttgagaaaatcactggcaatcggacttcagagctgcgggcacaggtgtggttagaactgagatacgacctgcccacctgggtcaggcctaaagacaagaagtcctgagttcttgccactgagtaggccagggtcatttgtccagaaaactttgtgactgtctttgagtgacctagtctgggacccattcattggtgggttctaaggttagaagctcatccaggatattttcaatattaagtcagtgcatagctgcaccactaacaaattggtgcctgtagagtcagagtgggtcaattcttaggacaatggcgctggcactgttagaggactggtgcaggataatgagtgtggatgagcagaagtcactgatggttacggggataccggcggactttgaggaggctgagattcaggaggtccttcaggagactttaaagtctctgggcaggtatagactgcttggcaagatattccggaagcaggagaatgccaatgctgtcttactagagcttctggaagatactgatgtctcggccattcccagtgaggtccagggaaaggggggtgtctggaaggtgatctttaagacccctaatcaggacactgagtttcttgaaagattgaacctgtttctagaaaaagaggggcagacggtctcgggtatgtttcgagccctggggcaggagggcgtgtctccagccacagtgccctgcatctcaccagaattactggcccatttgttgggacaggcaatggcacatgcgcctcagcccctgctacccatgagataccggaaactgcgagtattctcagggagtgctgtcccagccccagaggaagagtcctttgaggtctggttggaacaggccacggagatagtcaaagagtggccagtaacagaggcagaaaagaaaaggtggctggcggaaagcctgcggggccctgccctggacctcatgcacatagtgcaggcagacaacccgtccatcagtgtagaagagtgtttggaggcctttaagcaagtgtttgggagcctagagagccgcaggacagcccaggtgaggtatctgaagacctatcaggaggaaggagagaaggtctcagcctatgtgttacggctagaaaccctgctccggagagcggtggagaaacgcgccatccctcggcgtattgcggaccaggtccgcctggagcaggtcatggctggggccactcttaaccagatgctgtggtgccggcttagggagctgaaggatcagggcccgccccccagcttccttgagctaatgaaggtaatacgggaagaagaggaggaagaggcctcctttgagaatgagagtatcgaagagccagaggaacgagatggctatggccgctggaatcatgagggagacgactgaaaaccacctgggggcaggacccacagccagtgggctaagacctttaaaaaatttttttctttaatgtatgggactgaaatcaaaccatgaaagccaattattgaccttccttccttccttccttccctcccttcctccttctctccttctctcctcctctctcctctcctctcctctctttccttccttccttccttttttctttttctctttcttctttatttcttgggtctcactctcatcacccaggctagagtgcagtggcacaaaaatctcggctcactgcagccttgacttcccaggctcaggctcaggtgatcctcacaccttagcctcccaagtacctgggactacaggcacgcaccaccatgcctagctattcttttgtatttttggtagagacagggttttgctgtgttgctcaggctggtctggaacccctaggctcaaatgatgtgcccaactcggcctcccaaagtgctgggattacaggcatgaaccgccatgcctggcccttgatttttctttttaagaaaaaaatatctaggagtttcttagaccctatgtagattattaatgaacaaaagattaaactccaaatattaaatagtaagcctgaaggaatctgaaacacttgtacttccaattttctttaaataatcccaaatagaccagaattggcccataccatagaagaaagaattggcagtcaaaaaaaaaaataccttttgtaatgtttgaaaaataaagctgtttgacttgtcaggtgttttcctttctcaaatcagcaaattctctctgagtgcctggctttgtgagacactgtacaaggagttacaagactacagctataacctgcagttgagcagttataaacctacaaaatgggccctgccctcagagaggttccagtctagatgaggagctgatctagacaggtaaaaggctaactaaccctttgtgtaaataagttcatcaccccagtaaaagtgtcatcacccagtgaataggaccacctctgcctgcagatttttgttgttgttgttgtcattgttgttgttgttttaacctgggaagtgttcttcctgcctttctgctaggtgtcagatagatggtcccagagctaggtgctgtgtcaggccctgaagacacagatgactcaacctaagctttactttccagaggtccacagcctgagaggtgtccccaaagaaagggggacatgaggggactgcatgcttgagagcagggttgtttagggcaggtttggatttagtgagcaggctggtttgcttagagaaggcttttagtggcaacaaaggatgaagaggagagaaaaggaactcacatttattgagggcctactgtgtgcaaagtgtttcatgtatatctcattgaatgtatacagccaccctgttgtggtataattttgctctttataaagagaaagaccgaagctcagatgagttaagtggtctcctcaacaccaaaatgccaagaagtgatggagcctagacagaagcccagaactttctgactcacactagtccatcctctaccatcacgatgactttcaaattgtgctctgcagttctgcagattttctagcagtgccatctccaaaatgtgttttaaactctttatttttttaattattattagtattattttgagactgagtcttgctctatcacccaggctggagtgcagtggtgcaatctcagctcactgcaacctccgcctcccaggttcaagcgatttcgtgcctcagcctcccgagtagctgggattacaggcacccaccaccacgcccagctaatttttgtatttttagtagaaatggggtttcaccatgttggccaggctggtctcgaactcctgacctcaagtgatccactcacctcggcctcccaaagtgctgggattacaggtgtgagccaccatgcctgggctaaactctttaagtctctagtaaatgcagctagattcaaatgggctgataaccaaattttaacacatcagcattcaccaccaggtttacttttattttcagattggctcattttgtgcagaccttagagcaaagtttcctttatggtatctgtgtacgtatccaaacttcttttaattgttcacagattttaaaagcggtagcaccacatggttgtgtagatcagacctgtgtatttagatcagacctgtgtatcacgtaagtgtgtgagtgcagtgcagatgagcaccatttagttatatgtgctaggcaaatctccaacacagttgatgtgtagtcttgtggtagatttgtgcatactgtaagcaaattgcttagcttctctagacatcagtttccacatctgaaaaataagaagatgagagtacacggttgttatgaacaaatgacttaatgctttttaagcacgttgcatgacatctggaacacagaaagccctcaatacattgaagctcttaggattttcacgatgttcctgtctgctcaatgcatgctttctttattgttctgacagttgtgtggtaacaagctaatatgcttccagttgacttccagtctaccctggtgttagaaaccgtttcatctcttattgtaaatttgagtgcttgttgttttttatatttgtgatgactcttccagcagttgttgacaattgttagaggtttgacttttaaataattacttattttttctgattgtggttcagtttaactgaagaatatcctgagattgtaagaaaagcattttttaaaaggtatcacttgtgatcatttatctttctaaattctatttttaatactgttccaccaaagtgatgcagtggttaccatgacaccctaatttcatgtgtttttgtatttatgaaaatagtttcattgtcatttattggcggtatacaaagtaaaatgttataaatgtgaagttataaaataaatatatgctaataaaatcctgagtttttctgtttcct

SEQ ID NO: 9 - DNA, 1204 bp, Homo sapiens
ggggtgcaaagaagagacagcagcgcccagcttggaggtgctaactccagaggccagcatcagcaactgggcacagaaaggagccgcctgggcagggaccatggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcatccttgtgatcttctctggaatgttccttgttttcaccctggccggggccctgttcctccatcaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccctgagccagcacctgcggtagctgcactacagccctggcctccacccccaccccgccgaccatccaagggagagtgagacctggcagccacaactgcagtcccatcctcttgtcagggccctttcctgtgtacacgtgacagagtgccttttcgagactggcagggacgaggacaaatatggatgaggtggagagtgggaagcaggagcccagccagctgcgcctgcgctgcaggagggcgggggctctggttgtaaaacacacttcctgctgcgaaagacccacatgctacaagacgggcaaaataaagtgacagatgaccacc

SEQ ID NO: 10 - DNA, 459 bp, Homo sapiens
atccattctcctcatccctctgctctctggcctccagcctcccagcagcatggctttcaccggcaagttcgagatggagagtgagaagaattatgatgagttcatgaagctccttgggatctccagcgatgtaatcgaaaaggcccgcaacttcaagatcgtcacggaggtgcagcaggatgggcaggacttcacttggtcccagcactactccgggggccacaccatgaccaacaagttcactgttggcaaggaaagcaacatacagacaatggggggcaagacgttcaaggccactgtgcagatggagggcgggaagctggtggtgaatttccccaactatcaccagacctcagagatcgtgggtgacaagctggtggaggtctccaccatcggaggcgtgacctatgagcgcgtgagcaagagactggcctaagcagccaggcccggcccagggag

SEQ ID NO: 11 - DNA, 2775 bp, Homo sapiens
aatctttaggatctgagcaggagaaataccagcggatcttccccactctgctcccttccattcccacccttccttctttaataagcaggagcgaaaaagacaaattccaaagaggattgttcagttcaagggaatgaagaattcagaataattttggtaaatggattccaatatggggaataagaataagctgaacagttgacctgctttgaagaaacatactgtccatttgtctaaaataatctataacaaccaaaccaatcaaaatgaattcaacattattttcccaggttgaaaatcattcagtccactctaatttctcagagaagaatgcccagcttctggcttttgaaaatgatgattgtcatctgcccttggccatgatatttaccttagctcttgcttatggagctgtgatcattcttggtgtctctggaaacctggccttgatcataatcatcttgaaacaaaaggagatgagaaatgttaccaacatcctgattgtgaacctttccttctcagacttgcttgttgccatcatgtgtctcccctttacatttgtctacacattaatggaccactgggtctttggtgaggcgatgtgtaagttgaatccttttgtgcaatgtgtttcaatcactgtgtccattttctctctggttctcattgctgtggaacgacatcagctgataatcaaccctcgagggtggagaccaaataatagacatgcttatgtaggtattgctgtgatttgggtccttgctgtggcttcttctttgcctttcctgatctaccaagtaatgactgatgagccgttccaaaatgtaacacttgatgcgtacaaagacaaatacgtgtgctttgatcaatttccatcggactctcataggttgtcttataccactctcctcttggtgctgcagtattttggtccactttgttttatatttatttgctacttcaagatatatatacgcctaaaaaggagaaacaacatgatggacaagatgagagacaataagtacaggtccagtgaaaccaaaagaatcaatatcatgctgctctccattgtggtagcatttgcagtctgctggctccctcttaccatctttaacactgtgtttgattggaatcatcagatcattgctacctgcaaccacaatctgttattcctgctctgccacctcacagcaatgatatccacttgtgtcaaccccatattttatgggttcctgaacaaaaacttccagagagacttgcagttcttcttcaacttttgtgatttccggtctcgggatgatgattatgaaacaatagccatgtccacgatgcacacagatgtttccaaaacttctttgaagcaagcaagcccagtcgcatttaaaaaaatcaacaacaatgatgataatgaaaaaatctgaaactacttatagcctatggtcccggatgacatctgtttaaaaacaagcacaacctgcaacatactttgattacctgttctcccaaggaatggggttgaaatcatttgaaaatgactaagattttcttgtcttgctttttactgcttttgttgtagttgtcataattacatttggaacaaaaggtgtgggctttggggtcttctggaaatagttttgaccagacatctttgaagtgctttttgtgaatttatgcatataatataaagacttttatactgtacttattggaatgaaatttctttaaagtattactattaactgacttcagaagtacctgccatccaatacggtcattagattgggtcatcttgattagattagattagattagattgtcaacagattgggccatccttactttatgataggcatcattttagtgtgttacaatagtaacagtatgcaaaagcagcattcaggagccgaaagatagtctgaagtcattcagaagtggtttgaggtttctgttttttggtggtttttgtttgttttttttttttttcaccttaagggaggatttaatttgctcccaactgattgtcacttaaatgaaaatttaaaaatgaataaaaagacatacttctcagctgcaaatattatggagaattggggcacccacaggaatgaagagagaaagcagctccctaacttcaaaaccattttggtacctgacaacaagagcattttagagtaattaatttaataaagtaaattagtattgctgcaaatagttaaattatatttatttgaattgatggtcaagagattttccattttttttacagactgttcagtgtttgtcaagctttctggcataaatatgtactcaaaaggcatttccgcttacaatttgtagaaacacaaaatgcgttttccatacagcagtgcctatatagtgactgatttttaactttcaatgtccatctttcaaaggaagtaacaccaaggtacaatgttaaaggaatattcactttacctagcagggaaaaatacacaaaaactgcagatacttcatatagcccattttaacttgtataaactgtgtgacttgtggcgtcttataaataatgcactgtaaagattactgaatagttgtgtcatgttaatgtgcctaatttcatgtatcttgtaatcatgattgagcctcagaatcatttggagaaactatattttaaagaacaagacatacttcaatgtattatacagataaagtattacatgtgtttgattttaaaagggcggacattttattaaaatcaatattgtttttgctttttcaaaaaaaaaaaaaaaa

SEQ ID NO: 12 - DNA, 913 bp, Homo sapiens
ccagagaggggcaggctggtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctgctttggtcccattggtcgcgggcttggtgatctgcctcgtggtgtgcatccagcgcttcgcacaggctcagcagcagctgccgctcgagtcacttgggtgggacgtagctgagctgcagctgaatcacacaggacctcagcaggaccccaggctatactggcaggggggcccagcactgggccgctccttcctgcatggaccagagctggacaaggggcagctacgtatccatcgtgatggcatctacatggtacacatccaggtgacgctggccatctgctcctccacgacggcctccaggcaccaccccaccaccctggccgtgggaatctgctctcccgcctcccgtagcatcagcctgctgcgtctcagcttccaccaaggttgtaccattgcctcccagcgcctgacgcccctggcccgaggggacacactctgcaccaacctcactgggacacttttgccttcccgaaacactgatgagaccttctttggagtgcagtgggtgcgcccctgaccactgctgctgattagggttttttaaattttattttattttatttaagttcaagagaaaaagtgtacacacaggggccacccggggttggggtgggagtgtggtggggcgtagtggtggcaggacaagagaaggcattgagctttttctttcattttcctattaaaaaatacaaaaatca

SEQ ID NO: 13 - DNA, 63 bp, Artificial Sequence
  Description of Artificial Sequence Synthetic primer
ggccagtgaattgtaatacgactcactatagggaggcggtttttttttttttttttttttttt

SEQ ID NO: 14 - DNA, 20 bp, Artificial Sequence
  Description of Artificial Sequence Synthetic primer
cttggcatctgtattgtggt

SEQ ID NO: 15 - DNA, 20 bp, Artificial Sequence
  Description of Artificial Sequence Synthetic primer
gggtggccatggtcccaagg

SEQ ID NO: 16 - DNA, 20 bp, Artificial Sequence
  Description of Artificial Sequence Synthetic primer
atcagcttcctcctgtccct

SEQ ID NO: 17 - DNA, 20 bp, Artificial Sequence
  Description of Artificial Sequence Synthetic primer
gggctgcacttcgtgtgggt

SEQ ID NO: 18 - DNA, 20 bp, Artificial Sequence
  Description of Artificial Sequence Synthetic primer
tagcagacccctcagcacca

SEQ ID NO: 19 - DNA, 20 bp, Artificial Sequence
  Description of Artificial Sequence Synthetic primer
agcttcccgccctccatctg

SEQ ID NO: 20 - DNA, 20 bp, Artificial Sequence
  Description of Artificial Sequence Synthetic primer
actcctgcacgacctgctcc

SEQ ID NO: 21 - DNA, 20 bp, Artificial Sequence
  Description of Artificial Sequence Synthetic primer
ttatcacatgcttctggatg

SEQ ID NO: 22 - DNA, 20 bp, Artificial Sequence
  Description of Artificial Sequence Synthetic primer
tcactggaatcttccaaatt

SEQ ID NO: 23 - DNA, 20 bp, Artificial Sequence
  Description of Artificial Sequence Synthetic primer
gtttgagtttgggattttgg

SEQ ID NO: 24 - DNA, 20 bp, Artificial Sequence
  Description of Artificial Sequence Synthetic primer
atggatgcggagcggagggc

SEQ ID NO: 25 - DNA, 20 bp, Artificial Sequence
  Description of Artificial Sequence Synthetic primer
ctccacagcctcctggtctc

SEQ ID NO: 26 - DNA, 20 bp, Artificial Sequence
  Description of Artificial Sequence Synthetic primer
atgccggaggagggttcggg

SEQ ID NO: 27 - DNA, 20 bp, Artificial Sequence
  Description of Artificial Sequence Synthetic primer
cgtagctgccccttgtccag

SEQ ID NO: 28 - DNA, 20 bp, Artificial Sequence
  Description of Artificial Sequence Synthetic primer
gaggatctacctggagagga

SEQ ID NO: 29 - DNA, 20 bp, Artificial Sequence
  Description of Artificial Sequence Synthetic primer
ctggaagcccaggagttcca

SEQ ID NO: 30 - DNA, 20 bp, Artificial Sequence
  Description of Artificial Sequence Synthetic primer
tttcatccatccgacattga

SEQ ID NO: 31 - DNA, 20 bp, Artificial Sequence
  Description of Artificial Sequence Synthetic primer
atcttcaaacctccatgatg

SEQ ID NO: 32 - protein, 354 aa, Homo sapiens
MPRRSLHAAAVLLLVILKEQPSSPAPVNGSKWTYFGPDGENSWSKKYPSCGGLLQSPIDLHSDILQYDASLTPLEFQGYNLSANKQFLLTNNGHSVKLNLPSDMHIQGLQSRYSATQLHLHWGNPNDPHGSEHTVSGQHFAAELHIVHYNSDLYPDASTASNKSEGLAVLAVLIEMGSFNPSYDKIFSHLQHVKYKGQEAFVPGFNIEELLPERTAEYYRYRGSLTTPPCNPTVLWTVFRNPVQISQEQLLALETALYCTHMDDPSPREMINNFRQVQKFDERLVYTSFSQVQVCTAAGLSLGIILSLALAGILGICIVVVVSIWLFRRKSIKKGDNKGVIYKPATKMETEAHA

SEQ ID NO: 33 - protein, 459 aa, Homo sapiens
MAPLCPSPWLPLLIPAPAPGLTVQLLLSLLLLVPVHPQRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA

SEQ ID NO: 34 - protein, 239 aa, Homo sapiens
MPLGHIMRLDLEKIALEYIVPCLHEVGFCYLDNFLGEVVGDCVLERVKQLHCTGALRDGQLAGPRAGVSKRHLRGDQITWIGGNEEGCEAISFLLSLIDRLVLYCGSRLGKYYVKERSKAMVACYPGNGTGYVRHVDNPNGDGRCITCIYYLNKNWDAKLHGGILRIFPEGKSFIADVEPIFDRLLFFWSDRRNPHEVQPSYATRYAMTVWYFDAEERAEAKKKFRNLTRKTESALTED

SEQ ID NO: 35 - protein, 63 aa, Homo sapiens
MKHVLNLYLLGVVLTLLSIFVRVMESLEGLLESPSPGTSWTTRSQLANTEPTKGLPDHPSRSM

SEQ ID NO: 36 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 37 - protein, 174 aa, Homo sapiens
MLRTESCRPRSPAGQVAAASPLLLLLLLLAWCAGACRGAPILPQGLQPEQQLQLWNEIDDTCSSFLSIDSQPQASNALEELCFMIMGMLPKPQEQDEKDNTKRFLFHYSKTQKLGKSNVVSSVVHPLLQLVPHLHERRMKRFRVDEEFQSPFASQSRGYFLFRPRNGRRSAGFI

SEQ ID NO: 38 - protein, 159 aa, Homo sapiens
MLLLLLSIIVLHVAVLVLLFVSTIVSQWIVGNGHATDLWQNCSTSSSGNVHHCFSSSPNEWLQSVQATMILSIIFSILSLFLFFCQLFTLTKGGRFYITGIFILAGLCVMSAAAIYTVRHPEWHLNSDYSYGFAYILAWVAFPLALLSGVIYVILRKRE

SEQ ID NO: 39 - protein, 364 aa, Homo sapiens
MALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEASFENESIEEPEERDGYGRWNHEGDD

SEQ ID NO: 40 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSP

SEQ ID NO: 41 - protein, 128 aa, Homo sapiens
MAFTGKFEMESEKNYDEFMKLLGISSDVIEKARNFKIVTEVQQDGQDFTWSQHYSGGHTMTNKFTVGKESNIQTMGGKTFKATVQMEGGKLVVNFPNYHQTSEIVGDKLVEVSTIGGVTYERVSKRLA

SEQ ID NO: 42 - protein, 384 aa, Homo sapiens
MNSTLFSQVENHSVHSNFSEKNAQLLAFENDDCHLPLAMIFTLALAYGAVIILGVSGNLALIIIILKQKEMRNVTNILIVNLSFSDLLVAIMCLPFTFVYTLMDHWVFGEAMCKLNPFVQCVSITVSIFSLVLIAVERHQLIINPRGWRPNNRHAYVGIAVIWVLAVASSLPFLIYQVMTDEPFQNVTLDAYKDKYVCFDQFPSDSHRLSYTTLLLVLQYFGPLCFIFICYFKIYIRLKRRNNMMDKMRDNKYRSSETKRINIMLLSIVVAFAVCWLPLTIFNTVFDWNHQIIATCNHNLLFLLCHLTAMISTCVNPIFYGFLNKNFQRDLQFFFNFCDFRSRDDDYETIAMSTMHTDVSKTSLKQASPVAFKKINNNDDNEKI

SEQ ID NO: 43 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 44 - protein, 11 aa, Homo sapiens
LLGISSDVIEK

(19) United States
(12) Patent Application Publication
(10) Pub. No.: US 2010/0222230 A1
(43) Pub. Date: Sep. 2, 2010

(54) DIAGNOSTIC AND PROGNOSTIC METHODS FOR RENAL CELL CARCINOMA

(75) Inventors: Othon Iliopoulos, Cambridge, MA (US); Peter Hulick, Boston, MA (US)

Correspondence Address: DAVID S. RESNICK, NIXON PEABODY LLP, 100 SUMMER STREET, BOSTON, MA, 02110-2131, US

(73) Assignee: THE GENERAL HOSPITAL CORPORATION, Boston, MA (US)

(21) Appl. No.: 12/595,455

(22) PCT Filed: Apr. 11, 2008
(86) PCT No.: PCT/US08/60034

§ 371 Date: May 12, 2010

Related U.S. Application Data
(60) Provisional application No. 60/922,881, filed on Apr. 11, 2007.
(60) Provisional application No. 60/953,034, filed on Jul. 31, 2007.

(51) Int. Cl. C40B 30/04; C12Q 1/68; G01N 33/53; C40B 40/10

(52) U.S. Cl. 506/9; 435/6; 435/7.92; 506/18

(57) ABSTRACT

The present invention provides methods for diagnosis and prognosis of renal cell carcinoma (RCC) using expression analysis of one or more groups of genes, and a combination of expression analysis from a biological sample from the subject. The methods of the invention provide a method for superior detection accuracy for RCC as compared to any other currently available method for RCC diagnostic or prognosis. The invention also provides kits for diagnosis and prognosis of RCC using expression analysis.

### CROSS REFERENCED APPLICATIONS

This application claims benefit under 35 U.S.C. 119(e) of U.S. Provisional Patent Application Ser. No. 60/922,881 filed on Apr. 11, 2007 and U.S. Provisional Patent Application Ser. No. 60/953,034 filed on Jul. 31, 2007, the contents of which are incorporated herein in their entity by reference.

### GOVERNMENT SUPPORT

The present application was supported by the National Institutes for Health (NIH) Grant No 5 P50 CA101942-03, and the Government of the United States has certain rights thereto.

### FIELD OF THE INVENTION

The present invention relates generally to methods for diagnostic and prognostic methods of renal cell carcinoma (RCC) by analysis of gene group expression patterns in subjects. More specifically, the invention is directed to diagnostic and prognostic methods for detecting renal cell carcinoma in subjects by analysis of gene group expression patterns in subjects, preferably human subjects.

### BACKGROUND

Kidney cancer is a heterogeneous disease consisting of various subtypes with diverse generic, biochemical and morphologic features. Epithelial renal cell carcinoma (RCC) is the most common adult renal neoplasm, accounting for the vast majority (˜80%) of renal malignancies in adults. Based on morphological features defined in the WHO International Histological classification of Kidney Tumors, RCC can be divided into clear cell (conventional), papillary RCC (chromophil) (10-15%), chromophobe RCC (5%), collecting duct RCC (<1%) and unclassified RCC (<2%) subtypes.

Renal cell carcinoma (RCC) accounts for 2-3% of adult malignancies and its incidence is increasing. The most common histological subtype of RCC is conventional (clear cell) RCC, which accounts for 70-80% of all RCC cases. Many patients with von Hippel Lindau (VHL) disease, an autosomal dominant genetic disorder of inherited predisposition to RCC, also develop conventional RCC and studies on this familial disease facilitated the identification of the VHL tumor suppressor gene (Latif et al., Science, 1993; 260; 1317-1320).

The incidence of renal cell carcinoma (RCC) has steadily risen in the United States since 1970 and is currently estimated at approximately 51,000 cases per year. This increase has been observed across gender and race, increasing among black males and females by 3.9% and 4.3% per year, and white males and females by 2.3% and 3.1% per year, respectively [Jemal, 2007]. The majority of sporadic clear cell carcinoma is of clear cell histology (75%), followed by papillary Type I (5%) and Type II (5%), as well as chromophobe and oncocytoma (15%). It is clear that distinct molecular mechanisms underlie each histologic type [Iliopoulos, 2006].

Organ confined disease is treated with surgery and the five-year survival rate for patients presenting with Stage I disease is 95%, while the survival rate for patients with Stage II and III RCC is decreased to 70-80% and 40-60%, respectively [Motzer, 1996]. It is therefore reasonable to assume that early disease detection would improve overall survival in RCC patients.

VHL is now known to play a central role in the development of sporadic conventional RCC, with loss of heterozygocity being seen in the majority of tumors and mutations in more tan 50% of cases. Epigenetic silencing of VHL also occurs with promoter methylation being found in up to 20% of sporadic tumors.

Medical treatment of clear cell RCC patients has been evolving rapidly. Understanding of the VHL signaling pathway and its deregulation during clear cell RCC development has led to the identification of rational molecular therapeutic targets. Clinical trials with small molecule inhibitors of the vascular endothelial growth factor (VEGF), platelet derived growth factor (PDGF) and certain receptor or non-receptor cellular kinases shows promising results [Brugarolas, 2007; Escudier, 2007; Kane, 2006; Motzer, 2006; Motzer, 2007].

Targeted therapy has opened a new set of possibilities and questions in RCC treatment. Tumor response by classical imaging criteria fails to reflect changes in tumor vessel density, tumor viability, or correlate with disease progression or even overall survival. The availability of biomarkers that reflect disease activity may therefore help guide therapy. Biomarkers that serve as surrogate markers of tumor response will expedite a large number of clinical trials in which kinase inhibitor are used in combination in patients both pre and post surgery. Treatment of patients with minimal residual disease may prove, now that effective therapies are available, to be a better approach than treatment following clinical detection. Adjuvant trials may target patients with biomarker-detected minimal residual disease after nephrectomy for the primary tumor.

RCC is a histological diverse disease, with variable and often unpredictable clinical behavior. The prognosis worsens dramatically with the onset of clinical metastasis and current regimens of systematic therapy yield only modest benefits for metastatic RCC.

Surgical resection is the mainstay of therapy for patients with localized primary tumors. It is no exaggeration to say that new therapies are desperately needed for metastatic RCC, which is poorly responsive to chemotherapy and radiotherapy. Conventional treatment options currently available include: 1) treatment with small molecule inhibitors of receptor tyrosine kinases (inhibitors of vascular endothelial growth factor and/or platelet derived growth factor), 2) treatment with humanized antibody against vascular endothelial factor ligand), 3) mTOR inhibitors and 4) immunotherapy regimens that use interferon-α, interleukin 2, or both. The therapeutic benefits of immunotherapy are limited to a small percentage of patients with durable sustained complete remissions. A comprehensive meta-analysis of trials with at least one immunotherapeutic agent in one arm reported that immunotherapy yields an average response rate of 10.2%, a complete response rate of 3.2%, and a weighted average median survival improvement of 2.6 months. Patients treated with VEGF inhibitors have been reported to have a response rate of 40% but the effect is transient and eventually most of them progress.

Gene expression profiling could potentially be used to identify high-risk patients with localized RCC for early systemic therapy. Refining prognostic systems to more accurately predict patient outcomes and thereby guide more effective treatment decisions is an ongoing process. To date, key prognostic factors identified include TNM staging, tumor grade, functional status, and various biochemical assessments. Integrated prognostic systems have been developed by several groups combining clinical and pathological data to better stratify patients and improve prognostic power. Further integration of molecular markers defined by expression and proteomic profiling into these prognostic systems is likely to further increase prediction accuracy. Currently, there is no validated biomarker for renal cell cancer (RCC) such as PSA for prostate and CA125 for breast cancer. Currently there is no FDA approved marker for diagnosis of renal cell carcinoma.

Biomarker(s) that reliably correlate with disease burden or activity could be useful to detect disease before clinical signs and symptoms are apparent or even before there is radiological evidence of tumor growth. Such biomarkers can also be useful to guide early detection, such as techniques for detection of minimal residual disease (such as exploratory surgery or imaging), and could guide timing and choices of systemic therapy for relapsed or metastatic disease and can also be useful for the early identification of patients at need for adjuvant therapy after seemingly curative nephrectomy.

Such biomarkers could also be useful in the testing of potential therapeutic strategies for RCC. Surrogate markers of disease activity could also serve as surrogate endpoints in clinical trials and help shortening the length of a trial. Patients might avoid treatment with ineffective medications, thus preventing unnecessary side effect risks and serious complications.

RCC is not a uniform disease and is subdivided into clear cell, papillary, chromophobe and oncocytoma. Molecular genetic evidence indicates that the signaling pathways leading to the different histologic types are distinct. The majority of sporadic RCC (75%) are of clear cell type (REF). The earliest genetic defect underlying the generation of clear cell RCC is the loss of VHL tumor suppressor function and activation of its downstream target hypoxia inducible gene (HIF). Human renal cell carcinoma cell lines deficient in VHL and constitutively expressing HIF exist and they grow as tumors when transplanted in the flank of nude mice (REF). Reintroduction of the VHL gene or inactivation of HIF in these cell line suppress their growth as tumors in nude mice. This observation indicates that reintroduction of VHL and/or HIF inactivation in these cell lines may still regulate critical signaling pathways linked to tumor development.

Biomarkers for early diagnosis of RCC have the potential to guide therapeutic and preventive interventions, such as early administration of targeted/anti-angiogenic therapy, specialized imaging, exploratory surgery or chemoprevention trials. They can also serve as surrogate end-points in clinical trials. Unfortunately, reliable biomarkers for RCC have not been established yet.

### SUMMARY

The present invention provides compositions and methods for the diagnosis and prognosis of renal cell carcinoma (RCC) which provides a diagnostic test that is sensitive and specific.

The inventors have discovered a group of genes, herein termed “group of RCC biomarkers” that can be used for enhanced diagnosis and/or prognosis of renal cell carcinoma (RCC) in a subject. In some embodiments, the RCC biomarkers as disclosed herein are useful for enhanced diagnosis and/or prognosis of clear cell RCC in a subject. In some embodiments, the inventors have discovered that a subgroup of RCC biomarkers in the group of RCC biomarkers can be used for diagnosis and/or prognosis of renal cell carcinoma (RCC) in a subject. In some embodiments, RCC biomarkers are detected using gene expression analysis and in alternative embodiments, RCC biomarkers are detected by protein expression analysis.

The inventors provide detailed guidance on the increase and/or decrease of the gene expression and/or protein expression of the group of RCC biomarkers for the diagnosis and/or prognosis of RCC in a subject, and in some embodiments, for the diagnosis and/or prognosis of clear cell RCC in a subject.

One aspect of the present invention, the group of RCC biomarkers useful in the diagnosis and/or prognosis of RCC in a subject is set forth in Table 1. For example, the group of RCC biomarkers useful in the methods and compositions as disclosed herein comprise CA12; CA9; EGLN3; HIG2; TGFB3; NMU; PMP22; PNMA2; TNFRSF7; FABP6, CD70 (CD27L) and NPY1.

The inventors have further discovered that taking groups of genes from the group of RCC biomarkers, such as subgroup of RCC biomarkers from the group of biomarkers provides a much greater diagnostic and/or prognostic capability that chance alone. Preferably, a subgroup of RCC biomarkers comprises at least three RCC biomarkers from the group of RCC biomarkers set forth in Table 1. In some embodiments, a subgroup of RCC biomarkers comprises at least 4, at least 5, at least 6, at least 7, at least 8, at least 9, at least 10, at least 11 RCC biomarkers from the group of RCC biomarkers set forth in Table 1

It is noted that one can use any combination of RCC biomarkers set forth in Table 1 for subgroup of RCC biomarkers. In some instances, the inventors have discovered that one can enhance the accuracy of diagnosis by adding certain additional genes to the group of RCC biomarkers or a subgroup of RCC biomarkers as disclosed herein.

When one uses the group of RCC biomarkers or a subgroup of RCC biomarkers as disclosed herein, the expression of the group and/or a subgroup of RCC biomarkers in a biological sample from the subject are compared to the expression of the group and/or a subgroup of RCC biomarkers in a control biological sample. In some embodiments, the control biological sample can be normal tissue from the subject, or a biological sample from a subject that is not having with cancer, for example not having RCC.

One aspect of the present invention provides a method for identifying a subject having increased likelihood of developing or having renal cell carcinoma (RCC) the method comprising: (a) measuring the level of gene transcript expression or protein expression of a gene group wherein the gene group comprises at least three genes selected from a group of genes comprising; SEQ ID NO: 1, SEQ ID NO:2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO:12 in a biological sample obtained from a subject; and (b) comparing the level of gene transcript expression or protein expression of the genes as measured in step (a) to a reference level; wherein a higher level of the gene transcript expression or protein expression of the selected gene in the biological sample from the subject as compared the gene transcript expression or protein expression in the reference level indicates the subject is at increased risk of developing or having RCC.

Another aspect of the present invention provides a method for identifying a subject having increased likelihood of developing or having renal cell carcinoma (RCC) the method comprising: (a) measuring the level of gene transcript expression or protein expression of a gene group wherein the gene group comprises at least three genes selected from a group of genes encoding; CA12; CA9; EGLN3; HIG2; TGFB3; NMU; PMP22; PNMA2; TNFRSF7; FABP6; CD70 (CD27L); NPY1 in a biological sample obtained from a subject; (b) comparing the level of gene transcript expression or protein expression of the same genes as measured in the biological sample from the subject in step (a) to a reference level; wherein a higher level of the gene transcript expression or protein expression of the selected gene in the biological sample from the subject as compared the gene transcript expression or protein expression of the reference level indicates the subject is at increased risk of having or developing RCC.

Another aspect of the present invention provides a method for monitoring the progression of renal cell carcinoma (RCC) in a subject having, or likely of developing renal cell carcinoma (RCC), the method comprising: (a) measuring the level of gene transcript expression or protein expression of a gene group wherein the gene group comprises of at least three genes selected from a group of genes comprising; CA12; CA9; EGLN3; HIG2; TGFB3; NMU; PMP22; PNMA2; TNFRSF7; CD70 (CD27L); FABP6; NPY1 in a biological sample obtained from a subject at a first time point; (b) measuring the level of gene transcript expression or protein expression of at least three of the same genes as measured in step (a) in a biological sample obtained from a subject at a second time point; and comparing the level of gene transcript expression or protein expression of the same genes as measured in the biological sample from the first time point with the level of gene transcript expression or protein expression in the biological sample from the second timepoint; wherein a change in the level of the gene transcript expression or protein expression of at least three genes in the selected gene group in the biological sample from the subject at the first time point as compared to the level of gene transcript expression or protein expression of at least three of the same genes in the biological sample from the subject at the second timepoint indicates an alteration in the rate of progression of RCC in the subject. In such embodiments, if a decrease in the level of the gene transcript expression or protein expression from the first timepoint as compared to the second timepoint, it indicates in improved prognosis of RCC progression at the second timepoint as compared to the first timepoint. Alternatively, if an increase in the level of the gene transcript expression or protein expression from the first timepoint as compared to the second timepoint indicates in decreased prognosis of RCC progression at the second timepoint as compared to the first timepoint.

In some embodiments, the RCC biomarkers useful in the methods and compositions as disclosed herein are selected from a group of RCC biomarkers, for example at least 3, or at least 4, or at least 5, or at least 6, or at least 7, or at least 8, or at least 9 or at least 10, or at least 11 sequences of genes selected from the group consisting of GenBank identification Nos. or Unigene identification Nos: NM_{—}001218///NM_{—}017689///AF051882 (SEQ ID NO:1); NM_{—}001216///X66839 (SEQ ID NO:2); NM_{—}022073///NM_{—}033344///AJ310545 (SEQ ID NO:3); NM_{—}013332 (SEQ ID NO:4); NM_{—}003239 (SEQ ID NO:5); NM_{—}006681///X76029 (SEQ ID NO:6); NM_{—}000304///D11428 (SEQ ID NO:7); NM_{—}007257///XM_{—}376764 (SEQ ID NO:8); M63928///NM_{—}001033126///XM_{—}284241 (SEQ ID NO:9); U19869///NM_{—}001040442///NM_{—}001445 (SEQ ID NO:10); and NM_{—}000909 (SEQ ID NO:11); NM_{—}001252///L08096 (SEQ ID NO:12).

In some embodiments, a group of RCC biomarkers useful in the methods and compositions as disclosed herein comprises the sequences of genes with GenBank identification Nos. NM_{—}001218///NM_{—}017689///AF051882 (SEQ ID NO:1); NM_{—}001216///X66839 (SEQ ID NO:2); NM_{—}022073///NM_{—}033344///AJ310545 (SEQ ID NO:3); NM_{—}013332 (SEQ ID NO:4); NM_{—}003239 (SEQ ID NO:5); NM_{—}006681///X76029 (SEQ ID NO:6); NM_{—}000304///D11428 (SEQ ID NO:7); NM_{—}007257///XM_{—}376764 (SEQ ID NO:8); M63928///NM_{—}001033126///XM_{—}284241 (SEQ ID NO:9); U19869///NM_{—}001040442///NM_{—}001445 (SEQ ID NO:10); NM_{—}000909 (SEQ ID NO:11) and NM_{—}001252///L08096 (SEQ ID NO:12).

In some embodiments, a biological sample useful for measuring the level of RCC biomarker is serum, blood, plasma, urine and/or tissue sample. In alternative embodiments, a tissue sample is a biopsy tissue sample. In further embodiments, a biological sample is selected from a group of blood, serum, plasma, urine, stool, spinal fluid, sputum, nipple aspirates, lymph fluid, external secretions of the skin, respiratory tract, intestinal and genitourinary tracts, bile, saliva, milk, tumors, organs and also samples of in vitro cell culture constituents.

In some embodiments, the level of RCC biomarker can be determined by measuring the level of protein expression, for example by methods commonly known by person or ordinary skill in the art, for example where the protein expression is detected using an antibody, human antibody, humanized antibody, recombinant antibodies, monoclonal antibodies, chimeric antibodies, aptamer, peptide or analogues, or conjugates or fragments thereof. In some embodiments, proteins expression is detected by use of protein-binding molecules, such as in methods such as ELISA, or multiplex immuno assays.

In some embodiments, the level of RCC biomarker can be determined by measuring the level of gene transcript, for example at the level of messenger RNA (mRNA), for example by methods commonly known by person or ordinary skill in the art, such as but not limited to detection uses nucleic acid or nucleic acid analogues, such as, for example but not limited to nucleic acids and nucleic acid analogues such as DNA, RNA, PNA, pseudo-complementary DNA (pcDNA), locked nucleic acid and variants and homologues thereof. In some embodiments, detection of gene transcript level is assessed by reverse-transcription polymerase-chain reaction (RT-PCR).

Another aspect of the present invention provides a method for preventing the progression of renal cell carcinoma (RCC), the method comprising measuring the level of gene transcript expression or protein expression of a gene group wherein the gene group comprises of at least three genes selected from a group of genes comprising; CA12; CA9; EGLN3; HIG2; TGFB3; NMU; PMP22; PNMA2; PNMA2; TNFRSF7; FABP6; CD70 (CD27L); NPY1 in a biological sample and assessing the risk of a subject developing or having RCC according to claims 1-3, wherein a clinician directs the subject to be treated with an appropriate therapy if the subject has, or is at risk of developing RCC.

Another aspect of the present invention provides an array comprising a solid platforms, including nanochips and beads, such as disclosed in US Patent Application US2007/0065844, comprising in known positions on the array antisense nucleic acid sequences to fragments of at most 50 different genes, wherein at least three of the 50 genes are selected from of the genes SEQ ID NO: 1, SEQ ID NO:2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO:12.

Another aspect of the present invention provides an array comprising a solid platform comprising in known positions on the array antisense nucleic acid sequences to fragments of at most 100 different genes, wherein at least three of the 100 genes are selected from of the genes SEQ ID NO: 1, SEQ ID NO:2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO:12.

Another aspect of the present invention provides an array comprising a solid platforms, including nanochips and beads, such as disclosed in US Patent Application US2007/0065844, and attached the solid platform are protein-binding molecules, wherein the array comprises at most 50 different protein-binding molecules in known positions, wherein at least three of the 50 different protein-binding molecules have a specific binding affinity for proteins selected from the group of CA12; CA9; EGLN3; HIG2; TGFB3; NMU; PMP22; PNMA2; TNFRSF7; FABP6; CD70 (CD27L); NPY1. A protein-binding molecule useful for detection of a RCC biomarker protein as disclosed herein should have a specific binding affinity for at least one epitope on a RCC protein, or functional fragment or functional variants thereof. Protein-binding molecules, such as for example but not limited to, antibodies useful to detect RCC proteins as disclosed herein include protein-binding molecules with affinity for at least one of the following proteins; CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43), or functional fragments or variants thereof.

Another aspect of the present invention provides an array comprising a solid platform, including nanochips and beads, such as disclosed in US Patent Application US2007/0065844, and attached the solid platform are protein-binding molecules, wherein the array comprises at most 100 different protein-binding molecules in known positions, wherein at least three of the 100 different protein-binding molecules have a specific binding affinity for proteins selected from the group of CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43), or functional fragments or variants thereof.

In some embodiments, the arrays of the present invention are useful in methods to identify a subject having increased likelihood of developing or having renal cell carcinoma (RCC) according to the methods as disclosed herein.

Yet another aspect of the present invention relates to a kit comprising antisense nucleic acid sequences which have a substantial identity to a fragment of at least three genes selected from the group of: SEQ ID NO: 1, SEQ ID NO:2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO:12.

In some embodiments, the present invention provides a kit comprising antisense nucleic acid sequences which have a substantial identity to a fragment of at least four to six genes selected from the group of: SEQ ID NO: 1, SEQ ID NO:2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO:12.

In some embodiments, the present invention provides a kit comprising antisense nucleic acid sequences which have a substantial identity to a fragment of at least six to eight genes selected from the group of: SEQ ID NO: 1, SEQ ID NO:2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO:12.

In some embodiments, the present invention provides a kit comprising antisense nucleic acid sequences which have a substantial identity to a fragment of at least six to eight genes selected from the group of: SEQ ID NO: 1, SEQ ID NO:2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO:12.

In some embodiments, the present invention provides a kit comprising antisense nucleic acid sequences which have a substantial identity to a fragment of at least ten to twelve genes selected from the group of: SEQ ID NO: 1, SEQ ID NO:2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO:12.

Another aspect of the present invention provides a kit comprising protein-binding molecules, wherein at least three protein-binding molecules have specific binding affinity for at least three proteins selected from the group of CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43) or functional fragments or functional variants thereof. In some embodiments, the present invention provides a kit comprising protein-binding molecules, wherein at least four to six of the protein-binding molecules have specific binding affinity for at least four to six selected from the group of CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43) or functional fragments or functional variants thereof.

In another embodiment, the present invention provides a kit comprising protein-binding molecules, wherein at least six to eight of the protein-binding molecules have specific binding affinity for at least six to eight selected from the group of CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43) or functional fragments or functional variants thereof.

A kit comprising protein-binding molecules, wherein at least eight to ten of the protein-binding molecules have specific binding affinity for at least eight to ten selected from the group of CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43) or functional fragments or functional variants thereof.

In another embodiment, the present invention provides a kit comprising protein-binding molecules, wherein at least ten to eleven of the protein-binding molecules have specific binding affinity for at least ten to twelve selected from the group of CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43) or functional fragments or functional variants thereof.

In some embodiments, the present inventor provides a kit comprising protein-binding molecules to detect the RCC protein biomarkers as disclosed herein, for example a ELISA kit, or a multiplex immuno assay.

### BRIEF DESCRIPTION OF FIGURES

FIG. 1 shows a heat-map of VHL-HIF dependent genes expressed in isogenic cell lines. Gene expression differences between VHL deficient (PRC3) and VHL reconstituted (WT8) 786-0 RCC cell lines and cell lines 41, 76 and 77 as compared to DMSO control and blank (Blk) are defined as filter I. RNAi against HIF2A (PTR) recapitulates the genomic profiling signature of WT8, whereas the empty vector control recapitulates PRC3.

FIGS. 2A-2G shows signal dependency of candidate biomarkers QRT-PCR differential expression of candidate biomarkers in WT8, PRC3 PTV and PTR cell lines. FIG. 2A shows expression of CA12, FIG. 2B shows expression levels of EGLN3, FIG. 2C shows expression levels of FABP6 (WT8 and PRC3 cells only), FIG. 2D shows expression levels of HIG2, FIG. 2E shows expression levels of PNMA2, FIG. 2F shows expression levels of PMP22 and FIG. 2F shows expression levels of TNFSF7.

FIGS. 3A-3G show organ restricted expression of candidate biomarkers using QRT-PCR on RNA derived from select normal adult human tissues. FIG. 3A shows expression of CA12 in adult tissue, FIG. 3B shows expression of EGLN2 in adult tissue, FIG. 3C shows expression of FABP6 in adult tissue, FIG. 3D shows expression of HIG2 in adult tissue, FIG. 3E shows expression of PNMA2 in adult tissue, FIG. 3F shows expression of PMP22 in adult tissue, and FIG. 3G shows expression of TNFSF7 in adult tissue.

FIG. 4A-4F shows the upregulation of a single biomarkers in RCC tumors compared to normal matched renal tissue. FIG. 4A shows between approximately a 2-fold to about 11-fold upregulation of CA12 in RCC tumor (T) as compared to normal tissue (N), FIG. 4B shows between approximately a 2-fold to about 20-fold upregulation of EGLN3 in RCC tumor (T) as compared to normal tissue (N), FIG. 4C shows between approximately a 1-fold to about 10-fold upregulation of FABP6 in RCC tumor (T) as compared to normal tissue (N), FIG. 4D shows between approximately a 2.5-fold to about 28-fold upregulation of HIG2 in RCC tumor (T) as compared to normal tissue (N), FIG. 4F shows between approximately a 2-fold to about 8-fold upregulation of PMP22 in RCC tumor (T) as compared to normal tissue (N) and FIG. 4E shows between approximately a 2-fold to about 11-fold upregulation of PNMA2 in RCC tumor (T) as compared to normal tissue (N). Fold changes in the expression of each individual biomarker in a set of 10 tumor-normal tissue matched pairs through microarray analysis of cDNA from reverse transcribed ccRCC tumor/normal tissue RNA extracts. Tissue matched samples are tumor and normal tissue samples from the same subject.

FIG. 5A-5E show upregulation of biomarker set expression in RCC tumors compared to normal matched renal tissue. Biomarker profiling to determining levels of all 6 biomarkers in a given tumor specimen (T) as compared to its normal (N) matched tissue. FIGS. 5A, 5B, 5C, 5D, and 5E represent the expression of each of the different biomarkers; CA12, EGLN3, FABP6, HIG2, PMP22 and PNMA2 in 6 different subjects respectively. Tissue matched samples are tumor and normal tissue samples from the same subject. Matched control refers to use of tumor and normal tissue samples from the same subject.

FIG. 6 shows level of CA9 protein in the plasma from 5 subjects before (pre) and 1 month following (post) nephrectomy. The level of CA9 decreases in 80% (4 of 5) subjects assessed following nephrectomy.

FIGS. 7A-7B show the expression of PMP22 in tissue section from kidney in normal and tumor tissue. FIG. 7A shows PMP22 immunostaining in normal kidney as compared to increased PMP22 immunostaining in renal cell carcinoma tumor tissue as shown in FIG. 7B. (magnification ×20)

FIGS. 8A-8B shows expression of PMP22 in tissue section from kidney in normal and tumor tissue. FIG. 8A shows PMP22 immunostaining in normal kidney as compared to increased PMP22 immunostaining in renal cell carcinoma tumor tissue as shown in FIG. 8B. (magnification ×60).

FIG. 9A shows expression of carbonic anhydrase 9 (CA) in human renal cell carcinoma cell lines and tumors. FIG. 9A shows cell lysates of clones derived from human renal cell carcinoma cell lines 786-O, UMRC2 and UMRC6, stably transfected with vector control plasmid (lanes 1, 3 and 5) or plasmids expressing VHL30 (lanes 2a and 4) or VHL19 (lane 6) were immunoblotted for HIF1a, HIF2a, VHL and CA9 as indicated. Actin was used as loading control.

FIG. 10 shows QRT-PCR of CA9 message from the same cell lines, 786-O, UMRC2 and UMRC6 as shown in FIG. 1.

FIG. 11A-11B shows the expression of CA9 from QRT-PCR. FIG. 11A shows the fold increase of CA9 in clear cell human RCC tumor (T) compared to normal (N) matched kidney tissue. FIG. 11B shows the absolute values of expression of CA9 in RCC tumor (T) compared to normal (N) matched kidney tissue.

FIG. 12 shows the relative tissue expression of CA9 in adult human tissues.

FIG. 13A-13D shows the changes in plasma levels of CA9 in patients undergoing curative nephrectomy for localized clear cell RCC. FIG. 13A shows MGH patients: patient sex, disease stage, tumor volume and plasma levels of CA9 before (PRE) or after (POST) nephretomy are listed. FIG. 13A shows the correlation between tumor volume and pre-operative levels of CA9 in the MGH patient group. FIG. 13C shows the patient sex, disease stage, tumor volume and serum levels of CA9 before (PRE) or after (POST) nephretomy in the MD Anderson patient group are listed. FIG. 13D shows the correlation between tumor volume and pre-operative levels of CA9 in the serum of MDACC patient group.

FIG. 14A-14B shows blood CA9 levels in non clear cell kidney lesions and normal controls. FIG. 14A shows changes in plasma levels of CA9 in patients undergoing nephrectomy for benign renal lesions or RCC of non-clear histology. FIG. 14B shows a comparison of plasma levels of CA9 between RCC patients at presentation (RCC) and normal control individuals (NL). Horizontal bars indicate median values (in pg/ml).

FIG. 15 shows longitudinal measurements of plasma levels of CA9 in patients with clear cell RCC undergoing curative or debulking nephrectomy. SU=treatment with suten, G=treatment with gemcitabine, PR=partial response, SD=stable disease, DP=disease progression, NED=no evidence of disease.

FIGS. 16A-16C shows SRM-facilitated identification and quantification of FABP6 in cell lysates and tissue culture supernatant of VHL null and reconstituted cell lines. FIG. 16A shows quantification of FABP4 by SRM using peptide #4 (LLGISSDVIEK) (SEQ ID NOL 44), with values of: m/z 587.73>947.36, and a retention time of 35.07 min.

### DETAILED DESCRIPTION

The present invention provides compositions and methods for the diagnosis and prognosis of renal cell carcinoma (RCC) which provides a diagnostic test that is sensitive and specific.

The inventors have discovered a method that significantly increases the diagnostic accuracy of identifying a subject with an increased likelihood of having or developing renal cell carcinoma using gene expression analysis of a group of RCC biomarker genes. Accordingly, the inventors have discovered a method for enhanced diagnosis and/or prognosis of renal cell carcinoma (RCC) in a subject by assessing the expression level of a group of RCC biomarkers or a subgroup thereof in any combination, enabling dramatically improved detection of RCC in a subject and at an earlier stage than any available method to date.

As disclosed herein, the inventors have identified candidate biomarkers for RCC disease activity. First, the inventors identified the genes regulated by VHL, thus dependent on this specific signal transduction pathway. To narrow the pool of candidate biomarkers the inventors selected for the ones that expressed in a relatively restricted way in adult normal tissues, based on the fact that restricted adult tissue expression pattern will allow for larger tumor-dependent incremental blood level changes.

The inventors coined the abbreviation “SIDOR” for this “signal dependent and organ restrictive” algorithm which was used to discover RCC biomarkers as disclosed herein. The inventors herein demonstrate that use of the SIDOR algorithm can identify sensitive and specific subset of biomarkers for RCC. Specifically, the inventors have demonstrated using one of the RCC biomarkers identified, the fatty acid binding protein 6 (FABP6), as an exemplary example to quantify the levels of this protein in cell lysates and plasma of patients prior to and after nephrectomy for clinically localized RCC. The results as disclosed herein demonstrate that regulation of FABP6 message by VHL translates into differential protein levels in vitro. In addition, reduction of tumor mass in vivo is followed by a decline in plasma FABP6 levels. Taken together these data demonstrate that the translational SIDOR algorithm to identify cell signature differences is useful for rational development of candidate plasma biomarkers.

Using differential gene expression of VHL-deficient conventional RCC cells lines transfected with a VHL, the inventors have discovered VHL-associated changes in gene expression that accompany the development of RCC. The inventors discovered the expression of a group of genes was increased in VHL-deficient conventional RCC cells as compared to RCC cells comprising the VHL gene. The inventors validated the identified specific VHL-dependent gene expression changes by using restricted tissue expression analysis and protein expression analysis in RCC cell line pairs and renal tumor tissue. The inventors then selected those genes with restricted or tissue specific gene expression patterns, in other words, genes that were expressed normally only in a few tissue and/or types, such as genes that were expressed in a maximum of 2 or 3 organs such as the liver, kidney, brain. etc

Accordingly, the inventors have discovered that a subgroup of RCC biomarkers in the group of RCC biomarkers can be used for diagnosis and/or prognosis of renal cell carcinoma (RCC) in a subject. In some embodiments, RCC biomarkers are detected using gene expression analysis and in alternative embodiments, RCC biomarkers are detected by protein expression analysis.

In some embodiments, the group of RCC biomarkers or subgroup thereof in any combination can be detected at the level of gene expression, for example gene transcript level such as mRNA expression. In alternative embodiments, the group of RCC biomarkers or subgroup thereof in any combination can be detected at the level of protein expression.

The inventors provide detailed guidance on the increase and/or decrease of the gene expression and/or protein expression of the group of RCC biomarkers for the diagnosis and/or prognosis of RCC in a subject.

One aspect of the present invention, the group of RCC biomarkers useful in the diagnosis and/or prognosis of RCC in a subject is set forth in Table 1. For example, the group of RCC biomarkers useful in the methods and compositions as disclosed herein comprise CA12; CA9; EGLN3; HIG2; TGFB3; NMU; PMP22; PNMA2; TNFRSF7; FABP6; CD70 (CD27L) and NPY1. In particular embodiments, the group of RCC biomarkers is selected from the group consisting of CA9; EGLN3; HIG2; PNMA2; TNFRSF7; CD70 (CD27L) and FABP6.

The inventors have further discovered that taking groups of genes from the group of RCC biomarkers, such as subgroup of RCC biomarkers from the group of biomarkers provides a much greater diagnostic and/or prognostic capability that chance alone. Preferably, a subgroup of RCC biomarkers comprises at least two or at least three RCC biomarkers from the group of RCC biomarkers set forth in Table 1. In some embodiments, a subgroup of RCC biomarkers comprises at least 4, at least 5, at least 6, at least 7, at least 8, at least 9, at least 10, or at least 11 RCC biomarkers from the group of RCC biomarkers set forth in Table 1.

It is noted that one can use any combination of RCC biomarkers set forth in Table 1 for a subgroup of RCC biomarkers useful in the methods as disclosed herein. In embodiments, one can enhance the accuracy of diagnosis by adding additional genes to the group of RCC biomarkers listed in Table 1 or a subgroup thereof in any combination. In such embodiments, the additional genes can be any gene, for example other cancer biomarker genes, and in particular any other RCC biomarker gene that is not listed in Table 1.

TABLE 1
Group of RCC Biomarkers.

| | Accession | | Gene | Affymetrix |
|---|---|---|---|---|
| SEQ ID NO | number | Gene Title | Symbol | probe set |
| SEQ ID NO: 1 | NM_001218, | carbonic anhydrase XII | CA12 | 210735_s_at and |
| | NM_017689, | | | 203963_at |
| | AF051882 | | | |
| SEQ ID NO: 2 | NM_001216, | carbonic anhydrase IX | CA9, MN | 205199_at |
| | X66839 | | | |
| SEQ ID NO: 3 | NM_022073, | egl nine homolog 3 | EGLN3, | 219232_s_at |
| | NM_033344; | (C. elegans) | PHD3 | |
| | AJ310545 | | | |
| SEQ ID NO: 4 | NM_013332 | hypoxia-inducible | HIG2 | 218507_at |
| | | protein 2 | | |
| SEQ ID NO: 5 | NM_003239 | transforming growth | TGFB3 | 209747_at |
| | | factor, beta 3 | | |
| SEQ ID NO: 6 | NM_006681, | neuromedin U | NMU | 206023_at |
| | X76029 | | | |
| SEQ ID NO: 7 | NM_000304, | peripheral myelin | PMP22, HNPP, | 210139_s_at |
| | D11428 | protein 22 | GAS-3, Sp110 | |
| SEQ ID NO: 8 | NM_007257, | paraneoplastic antigen | PNMA2, MA2, | 209598_at and |
| | XM_376764 | MA2 | RGAG2; | 209597_s_at |
| | | | KIAA0883, MM2, | |
| SEQ ID NO: 9 | M63928; | tumor necrosis factor | TNFRSF7; | 206150_at |
| | NM_001033126, | receptor superfamily, | S152, Tp55, | |
| | XM_284241 | member 7 | CD27 | |
| SEQ ID NO: 10 | U19869, | fatty acid binding | FABP6, ILBP; | 210445_at |
| | NM_001040442, | protein 6, ileal | I-15P; I-BAP; | |
| | NM_001445; | (gastrotropin) | ILBP3; ILLBP; | |
| | | | I-BABP; I-BALB | |
| SEQ ID NO: 11 | NM_000909 | neuropeptide Y receptor | NPY1R, NPYR | 205440_s_at |
| | | Y1 | | |
| SEQ ID NO: 12 | NM_001252; | CD70 (CD27L) | TNFSF7, CD70, | |
| | L08096 | | CD27L, CD27LG, | |
| | | | CD27L | |

In some embodiments, one RCC biomarker useful in the compositions and methods as disclosed herein is Carbonic anhydrase 9 (CA9), which is a transmembrane protein contributing to the acidification of the extracellular environment, and is known to be a direct target of HIF. In some embodiments, a higher expression of CA9 as compared to a reference level may identify a subject with RCC responsive to IL-2 treatment.

In some embodiments, one of the RCC biomarker useful in the compositions and methods as disclosed herein is Carbonic anhydrase 12 (CA12), which is a similar biomarker to CA9. In some embodiments, a higher expression of CA12 of at least about 2-fold, or at least about 3-fold, or at least about 5-fold or at least about 7-fold or at least about 10-fold, or at least about 11-fold or greater than about 11-fold as compared to a reference level may identify a subject with a risk of having or developing RCC.

In some embodiments, one of the RCC biomarker useful in the compositions and methods as disclosed herein is Hypoxia inducible gene 2 (HIG2), which is a transcriptional target of HIF and beta catenin, and in some embodiments, over expression contributes to cellular transformation and growth of RCC cell lines in vitro. HIG2 protein typically localizes to the cell cytoplasm and the inventors have discovered HIG2 presence in tissue culture supernatant/plasma of RCC patients. In some embodiments, a higher expression of HIG2 of at least about 3-fold, or at least about 5-fold, or at least about 10-fold or at least about 15-fold or at least about 20-fold, or at least about 25-fold or greater than about 25-fold as compared to a reference level may identify a subject with a risk of having or developing RCC.

In some embodiments, one of the RCC biomarker useful in the compositions and methods as disclosed herein is Fatty acid binding protein 6 (FABP6), which is a direct target of HIF, and is expressed in the ileus only. FABP6 is actively secreted in the tissue culture supernatant. In some embodiments, a higher expression of FABP6 of at least about 1.5-fold, or at least about 2-fold, or at least about 3-fold or at least about 5-fold or at least about 7-fold, or at least about 10-fold or greater than about 10-fold as compared to a reference level may identify a subject with a risk of having or developing RCC.

In some embodiments, one of the RCC biomarker useful in the compositions and methods as disclosed herein is Peripheral myelin protein 22 (PMP22), which is known to be involved in Charco-Marie-Tooth peripheral demyelinating diseases, and expressed in adult peripheral and central nervous system. PMP22 is also a HIF target. PMP22 has multiple isoforms, therefore in some embodiments, a RCC biomarker useful in the compositions and methods as disclosed herein is a homologue or isoforms of PMP22. In some embodiments, a higher expression of PMP22 of at least about 1.5-fold, or at least about 2-fold, or at least about 3-fold or at least about 5-fold or at least about 7-fold, or at least about 8-fold or greater than about 8-fold as compared to a reference level may identify a subject with a risk of having or developing RCC.

In some embodiments, one of the RCC biomarker useful in the compositions and methods as disclosed herein is Paraneoplastic antigen 2 (PNMA2), which is a transmembrane protein involved in paraneoplastic limbic encephalopathy. In some embodiments, a higher expression of PNMA2 of at least about 1.5-fold, or at least about 2-fold, or at least about 3-fold or at least about 5-fold or at least about 10-fold, or at least about 12-fold or greater than about 12-fold as compared to a reference level may identify a subject with a risk of having or developing RCC.

In some embodiments, one of the RCC biomarker useful in the compositions and methods as disclosed herein is EGLN3. In some embodiments, a higher expression of PNMA2 of at least about 1.5-fold, or at least about 2-fold, or at least about 5-fold or at least about 10-fold or at least about 15-fold, or at least about 20-fold or greater than about 20-fold as compared to a reference level may identify a subject with a risk of having or developing RCC.

In some embodiments, one of the RCC biomarker useful in the compositions and methods as disclosed herein is Tumor necrosis factor (ligand) superfamily 7 (TNFSF7), which is a plasma circulating ligand with restricted adult tissue expression.

In one embodiment, the present invention provides gene groups the expression profile of which can be used in methods to diagnose renal cell carcinoma (RCC), such as clear cell RCC in more than 60%, preferably more than 65% still more preferably at least about 70% still more preferably about 75%, or still more preferably at about 80%-95% accuracy from a biological sample taken from the subject, for example a subject at risk of RCC.

Accordingly, the methods and compositions as disclosed herein provide gene groups that can be used in diagnosis and prognosis of RCC. Particularity, in one embodiment the present invention provides groups of genes the expression profile of which provides a diagnostic and/or prognostic test to determine RCC in a subject. For example, in one embodiment, the present invention provides groups of genes the expression profiles of which can distinguish subjects with RCC from subjects without RCC.

In one embodiment, the present invention provides early asymptomatic screening system for RCC by using analysis of the disclosed gene expression profiles. Such screening can be performed, for example in similar groups as colonoscopy for screening of colon cancer. Because early detection in RCC is crucial for efficient treatment, the gene expression analysis system of the present invention provides vastly improved methods to detect RCC that cannot yet be discovered by any other means currently available

When one uses the group of RCC biomarkers or a subgroup of RCC biomarkers as disclosed herein, the expression of the group and/or a subgroup of RCC biomarkers in a biological sample from the subject are compared to the expression of the group and/or a subgroup of RCC biomarkers to a reference level, for example a reference biological sample. In some embodiments, the reference level can be from a reference biological sample or a group of reference samples, for example such tissues can be normal tissue from the subject, or a biological sample from a subject that is not having with cancer, for example not having RCC.

As used herein the term “reference level” refers to the level of a RCC biomarker in at least one reference biological sample, or a group of biological samples from at least one normal subject or a group of normal subjects or, or subjects not with cancer, for example subjects not having or at risk of developing RCC. A reference level is normalized to 0%. An increase in the level of a RCC biomarker as compared with a reference level of the same RCC biomarker is at least 1% to 100% of the reference RCC biomarker level, including all percentages between 1% and 100%, i.e. at least 1%, 2%, 3%, 4%, 5%, 10%, 15%, 20%, 25%, 30%, 35%, 40%, 45%, 50%, 60%, 70%, 80%, 90%, 95%, 100%, or at least about 1.5-fold, or at least about 2-fold, at least about 3-fold, at least about 5-fold, at least about 10-fold, at least about 20-fold, or above about 20-fold increased as compared to the reference RCC biomarker level.

For example, the reference level for RCC biomarkers, such as CA12; CA9; EGLN3; HIG2; TGFB3; NMU; PMP22; PNMA2; TNFRSF7; FABP6; CD70 (CD27L) and NPY1, and in particular CA9; EGLN3; HIG2; PNMA2; TNFRSF7 and FABP6 are normalized to 0%. Higher levels of at least 3 RCC biomarkers selected from the group of CA12; CA9; EGLN3; HIG2; TGFB3; NMU; PMP22; PNMA2; TNFRSF7; FABP6; CD70 (CD27L) or NPY1 in the biological sample from the subject as determined by the methods as disclosed herein, for example a higher level by at least 1% to 100% as compared to the reference level, i.e. at least at least 1%, 2%, 3%, 4%, 5%, 10%, 15%, 20%, 25%, 30%, 35%, 40%, 45%, 50%, 60%, 70%, 80%, 90%, 95%, 100%, or at least about 1.5-fold, or at least about 2-fold, at least about 3-fold, at least about 5-fold, at least about 10-fold, at least about 20-fold, or higher level in the biological sample from the subject as compared to the reference level of CA12; CA9; EGLN3; HIG2; TGFB3; NMU; PMP22; PNMA2; TNFRSF7; FABP6; CD70 (CD27L) or NPY1 identifies a subject at risk of developing, or having RCC.

It should be noted, that the percentage increase for each RCC biomarker assessed of a group of RCC biomarkers assessed can be different, and the present invention encompasses identification of a subject at risk of developing, or having RCC if the level of each RCC biomarker tested in the biological sample increases by at least 1% as compared to the reference level for the same RCC biomarker.

As an exemplary example only, from a group of biomarkers tested in a biological sample from a subject, one RCC biomarker can be increased by 5%, a second RCC biomarker can be increased by 14% and a third RCC biomarker assessed can be increased by 1% as compared to the reference levels for each of the three RCC biomarker assessed, identifying a subject with increased risk of developing or having RCC.

In some embodiments, reference levels useful in the methods as disclosed herein can be biological samples obtained from a subject or a group of subjects who does not have cancer, in particular from a subject who does not have RCC or does not have a likelihood of developing RCC. In some embodiments, reference levels can be obtained from biological samples from the same subject, for example the reference level can be the level in a biological sample obtained from the subject at one time point, for example an earlier (i.e. first) time point, which us useful as a reference level for comparison with a biological sample from the same subject obtained at a later (i.e. second) time point. Such embodiments are useful for prognosis, for example monitoring RCC disease progression in a subject over a defined time period, for example from the time when the reference level (i.e. first biological sample) was obtained to the time when the second biological sample was obtained from the same subject. Such embodiments are useful to monitor disease progression of RCC in a subject, and in particular to monitor the disease progression of RCC in response to a therapy or anti-cancer therapy.

In some embodiments, reference levels useful in the methods as disclosed herein are obtained from a population group, which refers to a group of individuals or subjects sharing a common ethno-geographic origin. Reference levels can be reference levels from populations such as groups of subjects or individuals who are predicted to have representative levels of expression of the gene transcripts and/or proteins encoded by the RCC biomarkers listed in Table 1 found in the general population. Preferably, the reference level is from a population with representative levels of expression of the gene transcripts and/or proteins encoded by the RCC biomarkers listed in Table 1 in the population at a certainty level of at least 85%, preferably at least 90%, more preferably at least 95% and even more preferably at least 99%.

In another embodiment, the present invention provides a group of genes that can be used as predictors of RCC in a subject. These genes were identified using probabilities with a t-test analysis and show differential gene expression in subjects with RCC. A group of genes comprising between 1 and 11, and all combinations in between, for example, 2, 3, 4, 5, 6, 7, 8, 9, 10, 11 and 12 gene transcripts selected from the group consisting of genes selected from Table 1, and identified by the following GenBank Sequence Identification numbers (the identification numbers for each gene are separated by a “;” while alternative GenBank Sequence ID numbers are separated by “///”): NM_{—}001218///NM_{—}017689///AF051882 (SEQ ID NO:1); NM_{—}001216///X66839 (SEQ ID NO:2); NM_{—}022073///NM_{—}033344///AJ310545 (SEQ ID NO:3); NM_{—}013332 (SEQ ID NO:4); NM_{—}003239 (SEQ ID NO:5); NM_{—}006681///X76029 (SEQ ID NO:6); NM_{—}000304///D11428 (SEQ ID NO:7); NM_{—}007257///XM_{—}376764 (SEQ ID NO:8); M63928///NM_{—}001033126///XM_{—}284241 (SEQ ID NO:9); U19869///NM_{—}001040442///NM_{—}001445 (SEQ ID NO:10); NM_{—}000909 (SEQ ID NO:11) and NM_{—}001252///L08096 (SEQ ID NO:12) the expression profile of which can be used to diagnose RCC, for example clear cell RCC in a biological sample from a subject, when the expression pattern is compared to the expression pattern of the same group of genes in a control biological sample who does not have, or is not at risk of developing cancer, for example RCC.

In another embodiment, the gene/transcript analysis comprises a subgroup (subgroup) of about 3 to 5, 5 to 7, 7 to 9 or 9 to 12, or any integer in between, of any of the RCC biomarkers as shown in Table 1 or homologues thereof. In some embodiments, the subgroup of RCC biomarkers useful in the diagnostic and prognostic methods and compositions for RCC in a subject cam be combined with other biomarker genes, for example but not limited to other biomarker genes for cancer. In some embodiments, the group of RCC biomarkers or subgroup thereof in any combination can be combined with any number of other genes, for example other biomarker genes such as cancer biomarkers comprising a group of about 1, about 5, about 1-5, about 5-10, about 10-15, about 15-20, about 20-25, about 25-30 about 35-40 about 40-45 about 45-50 can be used to diagnose RCC, for example clear cell RCC in a biological sample from a subject, when the expression pattern is compared to the expression pattern of the same group of genes in a control biological sample who does not have, or is not at risk of developing cancer, for example RCC.

In one embodiment, the present invention provides a group of RCC biomarkers of which are increased in a subject having RCC, for example a subject with increased likelihood of developing RCC or a subject with RCC. In one embodiment, the group consists of at least 2 or at least 3 of RCC biomarker genes selected from the group consisting of: NM_{—}001218///NM_{—}017689///AF051882 (SEQ ID NO:1); NM_{—}001216///X66839 (SEQ ID NO:2); NM_{—}022073///NM_{—}033344///AJ310545 (SEQ ID NO:3); NM_{—}013332 (SEQ ID NO:4); NM_{—}003239 (SEQ ID NO:5); NM_{—}006681///X76029 (SEQ ID NO:6); NM_{—}000304///D11428 (SEQ ID NO:7); NM_{—}007257///XM_{—}376764 (SEQ ID NO:8); M63928///NM_{—}001033126///XM_{—}284241 (SEQ ID NO:9); U19869///NM_{—}001040442///NM_{—}001445 (SEQ ID NO:10); NM_{—}000909 (SEQ ID NO:11) and NM_{—}001252///L08096 (SEQ ID NO:12), or homologues or functional variants or fragments thereof.

In another embodiment, the present invention provides a methods for diagnosing whether a subject has RCC or if a subject has increased likelihood of developing RCC, the methods comprising obtaining nucleic acid from a biological sample from the subject and measuring the gene transcript levels of at least 2 or at least 3 RCC biomarkers selected from the group of RCC biomarkers listed in Table 1, and comparing the level of gene transcript of the same group of RCC biomarkers in a reference biological sample, wherein the difference in level of expression in the group of RCC biomarkers analyzed is indicative of the subject having an different risk of having or developing RCC as compared to the subject from which the reference biological sample was obtained. More specifically, an increased level of a group of at least 2 or at least 3 RCC biomarkers or more, preferably all of the RCC biomarkers listed in Table 1, in the biological sample from the subject as compared to the reference biological sample identifies the subject having, or having an increased risk of developing RCC. Alternatively, a decreased level of a group of at least 2 or at least 3 RCC biomarkers or more, preferably all of the RCC biomarkers listed in Table 1, in the biological sample from the subject as compared to the reference biological sample identifies the subject at decreased likelihood of having, or decreased likelihood of developing RCC as compared the subject from which the reference sample was obtained.

When the subject is identified to be at risk of developing RCC using the methods as disclosed herein, the subject may develop RCC in the near future or anytime in the future. Accordingly, such subjects can be selected for frequent follow up measurements of the levels of the gene transcripts of at least 2 or at least 3 RCC biomarkers as listed in Table 1 to allow early treatment of RCC. Alternatively, the present invention provides methods to diagnose subjects who are at a lesser risk of developing RCC by analyzing the gene transcript levels of at least 2 or at least 3 RCC biomarkers as listed in Table 1, to identify subjects not having or not at risk of RCC, which can be selected to not undergo as frequent follow up measurements of the levels of the gene transcripts of at least 2 or at least 3 RCC biomarkers as listed in Table 1, or other alternative invasive RCC diagnostic methods, as subjects identified with or at risk of developing RCC.

In some embodiments, the methods to measure the expression level of a group of RCC biomarkers or subgroups thereof as disclosed herein can measure the level of gene transcripts, such as mRNA expression. Methods to measure gene transcript levels are commonly known by persons of ordinary skill in the art, and are encompassed for use in the present invention, for example use nucleic acid hybridization methods. In some embodiments, methods to measure gene transcript levels, for example mRNA can use nucleic acid probes capable of hybridizing to the subject's gene/transcript sequences of the RCC biomarkers as disclosed herein. In some embodiments, methods to measure gene transcript expression can be nucleic acid probes that are immobilized on a surface, such as a nucleic acid binding chip to allow analysis diagnosis and prognosis by hybridizing to the subject's gene/transcript sequences of the RCC biomarkers as disclosed herein.

In alternative embodiments, the methods to measure the expression level of a group of RCC biomarkers or subgroups thereof as disclosed herein can measure the level of protein expression encoded by the RCC biomarker genes as disclosed herein. In some embodiments, protein-binding molecules with affinity for at least one of the proteins selected from the group of: CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43) or fragments or functional variants thereof are useful in the methods of the present invention. Methods to measure protein expression level are commonly known by persons of ordinary skill in the art, and are encompassed for use in the present invention, for example use of antibodies targeting the proteins encoded by the RCC biomarker genes. In some embodiments, methods to measure protein expression can use protein-binding molecules, for example antibodies or protein-binding agents that are immobilized on a surface, such as a protein chip to allow analysis diagnosis and prognosis by binding to the subjects the expressed proteins encoded by the RCC biomarkers as disclosed herein. In some embodiments, where the level of expression measured is the level of protein expression measured, protein expression can be measured using an antibody, human antibody, humanized antibody, recombinant antibodies, monoclonal antibodies, chimeric antibodies, alternative binding proteins, aptamer, peptide or analogues, or conjugates or fragments thereof. In some embodiments, protein expression can be measured by ELISA, by Multiplex Immuno-Assay methods and kits.

In another embodiment, the methods to measure the expression level of a group of RCC biomarkers or subgroups thereof as disclosed herein are performed by analyzing the level of proteins encoded by a group of RCC biomarkers or a subgroup thereof in any combination listed in Table 1 in a biological sample obtained from the subject.

In some embodiments, the biological sample is, for example but not limited to, urine, whole blood, plasma, serum, saliva, cell culture and tissue biopsies, scrapes (e.g. buccal scrapes) obtained from a subject.

In an alternative embodiment, methods to measure the expression level of a group of RCC biomarkers or subgroups thereof as disclosed herein can be performed using DNA by analyzing the gene expression regulatory regions of the group of RCC biomarkers or subgroups thereof as disclosed herein using nucleic acid polymorphisms, such as single nucleic acid polymorphisms (SNPs), where polymorphisms are known to be associated with increased and/or decreased expression are used to indicate increased or decreased expression of he gene transcript in the subject. For example, methylation patterns of the regulatory regions of the group of RCC biomarkers can be analyzed.

In some embodiments, the compositions comprise sets of probes that detect gene products encoded by the RCC biomarkers as disclosed herein, for example set of protein-binding agents having affinity and binding to proteins encoded by the RCC biomarkers and/or sets of nucleic acid probes that hybridize to gene transcripts encoded by the RCC biomarkers.

In some embodiments, a probe set can specifically bind and/or hybridize to at least one or all of the 12 gene products of RCC biomarkers as disclosed herein. In some embodiments, the probe set are capable or binding to and/or hybridizing to proteins, polypeptides or fragments thereof of RCC biomarkers as disclosed herein, such as CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43) or fragments, or functional variants or homologues thereof. In particular embodiments, probe sets are capable or binding to and/or hybridizing to proteins, polypeptides or fragments of CA9; EGLN3; HIG2; PNMA2; TNFRSF7; CD70 (CD27L) and FABP6 or variants or homologues thereof. In alternative embodiments, the probe sets are capable of specifically binding and/or hybridizing to at least one or all of the 12 gene products of RCC biomarkers, such as mRNA gene transcripts for CA12; CA9; EGLN3; HIG2; TGFB3; NMU; PMP22; PNMA2; TNFRSF7; FABP6; CD70 (CD27L) and NPY1 or variants or homologues thereof. In particular embodiments, probe sets are capable or binding to the mRNA or gene transcripts of CA9; EGLN3; HIG2; PNMA2; TNFRSF7; CD70 (CD27L) and FABP6 or variants or homologues thereof.

In another embodiment, the present invention provides methods and compositions for minimally invasive sample procurement methods for diagnosis and/or prognosis of RCC in a subject, by analyzing the group of RCC biomarkers or a subgroup thereof in any combination as disclosed herein by array-based gene expression profiling or measurement of the levels of protein encoded by the RCC biomarkers or subgroups thereof in biological sample from the subject. These methods can be used to diagnosis subjects who are already affected with RCC, such as clear cell RCC, or are at high risk of developing RCC. The methods as disclosed herein, in particular the methods described in the Examples for selecting differentially expressed genes for use as RCC biomarkers, on the basis of (i) increase expression in RCC samples without VHL as compared to RCC samples with VHL, and (ii) restricted tissue expression, can also be used to identify further patterns of gene expression and/or protein expression that are diagnostic of RCC, for example diagnostic for clear cell RCC, and to identify a subject at risk of developing RCC.

The invention further provides a group of RCC biomarkers on a microarray consisting of two or three or more of the RCC biomarkers as listed in Table 1, specifically indented for the diagnosis and/or prediction of RCC in a subject, or determining susceptibility of a subject to developing RCC.

In some embodiments, the present invention relates to a methods of diagnosing a RCC in a subject comprising obtaining a biological sample from a subject and obtaining the nucleic acid or protein from the sample to be diagnosed, and determining the expression of a group of identified genes such as the RCC biomarkers as disclosed herein in the biological sample, wherein a change in the expression of such RCC biomarkers in the biological sample from the subject as compared to the expression pattern of the same RCC biomarkers in a reference biological sample, such as that from a normal subject or health individual with a similar biometric profile (such as age, gender, ethnicity, lifestyle, weight etc) is indicative of a subject having a different likelihood of having or developing RCC as compared to the subject from which the reference sample was obtained. For example, an increase in the protein and/or gene transcript expression of at least 2 or at least 3 RCC biomarkers in the biological sample from the subject as compared to the protein and/or gene transcript expression of the same RCC biomarkers in the reference sample is indicative of the subject with a likelihood of having RCC or developing RCC as compared to the subject from which the reference sample was obtained.

Another aspect of the present invention relates to the use of the RCC biomarkers as disclosed herein for diagnostic and prognostic purposes to identify a subject at risk of, or having RCC. In another embodiment, the RCC biomarkers as disclosed herein can be used to monitor disease progression in a subject who has RCC. In another embodiment, the RCC biomarkers can be used to monitor therapeutic efficacy of an anti-cancer therapy in a subject with RCC. In another embodiment, the RCC biomarkers as disclosed herein can be used to assess effectiveness of drugs in human clinical trials for the treatment of RCC.

Determining Expression Level by Measuring mRNA

In an alternative embodiment, methods to measure the expression level of a group of RCC biomarkers or subgroups thereof as disclosed herein can be performed using DNA by analyzing the gene expression regulatory regions of the group of RCC biomarkers or subgroups thereof as disclosed herein using nucleic acid polymorphisms, such as single nucleic acid polymorphisms (SNPs), where polymorphisms are known to be associated with increased and/or decreased expression are used to indicate increased or decreased expression of he gene transcript in the subject. For example, methylation patterns of the regulatory regions of the group of RCC biomarkers can be analyzed.

In some embodiments, where the level of expression measured is the level of gene transcript expression measured, protein expression gene transcript expression can be measured at the level of messenger RNA (mRNA). In some embodiments, detection uses nucleic acid or nucleic acid analogues, for example, but not limited to, nucleic acid analogous comprise DNA, RNA, PNA, pseudo-complementary DNA (pcDNA), locked nucleic acid and variants and homologues thereof. In some embodiments, gene transcript expression can be assessed by reverse-transcription polymerase-chain reaction (RT-PCR) or quantitative RT-PCR by methods commonly known by persons of ordinary skill in the art.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In general, the PCR procedure describes a method of gene amplification which is comprised of (i) sequence-specific hybridization of primers to specific genes within a nucleic acid sample or library, (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

In an alternative embodiment, RCC biomarker levels can be determined by reverse-transcription (RT) PCR and by quantitative RT-PCR (QRT-PCR) or real-time PCR methods. Methods of RT-PCR and QRT-PCR are well known in the art, and are described in more detail below.

Real time PCR is an amplification technique that can be used to determine levels of mRNA expression. (See, e.g., Gibson et al., Genome Research 6:995-1001, 1996; Heid et al., Genome Research 6:986-994, 1996). Real-time PCR evaluates the level of PCR product accumulation during amplification. This technique permits quantitative evaluation of mRNA levels in multiple samples. For mRNA levels, mRNA is extracted from a biological sample, e.g. a tumor and normal tissue, and cDNA is prepared using standard techniques. Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, beta-actin) primers and probes can be obtained commercially from, for example, Perkin Elmer/Applied Biosystems (Foster City, Calif.). To quantitate the amount of the specific nucleic acid of interest in a sample, a standard curve is generated using a control. Standard curves can be generated using the Ct values determined in the real-time PCR, which are related to the initial concentration of the nucleic acid of interest used in the assay. Standard dilutions ranging from 10-10⁶ copies of the gene of interest are generally sufficient. In addition, a standard curve is generated for the control sequence. This permits standardization of initial content of the nucleic acid of interest in a tissue sample to the amount of control for comparison purposes.

Methods of real-time quantitative PCR using TaqMan probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994.

The TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq, results in the cleavage of the TaqMan probe. This cleavage separates the 5′ fluorescent dye and the 3′ quenching agent, thereby resulting in an increase in fluorescence as a function of amplification (see, for example, at the world-wide web site: “perkin-elmer-dot-com”).

In another embodiment, detection of RNA transcripts can be achieved by Northern blotting, wherein a preparation of RNA is run on a denaturing agarose gel, and transferred to a suitable support, such as activated cellulose, nitrocellulose or glass or nylon membranes. Labeled (e.g., radiolabeled) cDNA or RNA is then hybridized to the preparation, washed and analyzed by methods such as autoradiography.

Detection of RNA transcripts can further be accomplished using known amplification methods. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap lipase chain reaction (RT-AGLCR) as described by R. L. Marshall, et al., PCR Methods and Applications 4: 80-84 (1994). One suitable method for detecting enzyme mRNA transcripts is described in reference Pabic et. al. Hepatology, 37 (5): 1056-1066, 2003, which is herein incorporated by reference in its entirety.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 454-4610; strand displacement amplification (as described in G. T. Walker et al., Clin. Chem. 42: 9-13 (1996) and European Patent Application No. 684315; and target mediated amplification, as described by PCT Publication WO 9322461.

In situ hybridization visualization can also be employed, wherein a radioactively labeled antisense RNA probe is hybridized with a thin section of a biopsy sample, washed, cleaved with RNase and exposed to a sensitive emulsion for autoradiography. The samples can be stained with haematoxylin to demonstrate the histological composition of the sample, and dark field imaging with a suitable light filter shows the developed emulsion. Non-radioactive labels such as digoxigenin can also be used.

Alternatively, mRNA expression can be detected on a DNA array, chip or a microarray. In such an embodiment, probes can be affixed to surfaces for use as “gene chips.” Such gene chips can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the present invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayyem et al. U.S. Pat. No. 5,952,172 and by Kelley, S. O. et al. (1999) Nucleic Acids Res. 27:4830-4837

Oligonucleotides corresponding to RCC biomarker are immobilized on a chip which is then hybridized with labeled nucleic acids of a test sample obtained from a patient. Positive hybridization signal is obtained with the sample containing RCC biomarker mRNA transcripts. Methods of preparing DNA arrays and their use are well known in the art. (See, for example U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the tissue sample to be tested, reverse transcribed, and fluorescent-labeled cDNA probes are generated. The microarrays capable of hybridizing to RCC biomarker cDNA are then probed with the labeled cDNA probes, the slides scanned and fluorescence intensity measured. This intensity correlates with the hybridization intensity and expression levels.

Methods of “quantitative” amplification are well known to those of skill in the art. For example, quantitative PCR involves simultaneously co-amplifying a known quantity of a control sequence using the same primers. This provides an internal standard that can be used to calibrate the PCR reaction. Detailed protocols for quantitative PCR are provided, for example, in Innis et al. (1990) PCR Protocols, A Guide to Methods and Applications, Academic Press, Inc. N.Y.

### Determining Expression Level by Measuring Protein

In one embodiment, the levels of RCC biomarker can be determined by measuring the protein expression of the RCC biomarkers as disclosed herein. In some embodiments, protein expression can be measured by contacting a biological sample with an antibody-based binding moiety or protein-binding molecule that specifically binds to the protein of a RCC biomarker selected from the group of CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43) or a fragment or variant thereof. Formation of the antibody-RCC biomarker protein complex is then detected by a variety of methods known in the art.

In one embodiment, methods to detect the RCC proteins and fragments and functional variants thereof as disclosed herein include ELISA (enzyme linked immunosorbent assay), western blot, immunoprecipitation, immunofluorescence using detection reagents such as an antibody or protein binding molecules or protein-binding agents. Alternatively, a RCC protein biomarker can be detected in a subject by introducing into a subject a labeled anti-RCC biomarker antibody and other types of detection agent. For example, the antibody can be labeled with a radioactive marker whose presence and location in the subject is detected by standard imaging techniques, particularly useful are methods that detect a RCC protein or fragment thereof expressed in a subject or in a biological sample.

Methods to detect level the expression of RCC protein biomarker in a biological sample are well known to persons skilled in the art, and are encompassed for use in this invention. Commercially available antibodies and/or ELISA kits for detection of the expression of at least one or a combination of RCC protein biomarkers are also useful in the methods of this invention. Some examples of such protein-binding molecules useful to detect the RCC biomarker proteins are commercially available, and include, but are not limited to, commercially available antibodies from Cell Signalling Technologies (MA, USA), which can be found at world wide web site: “cellsignal-dot-com”. In some embodiments, antibodies from other antibody companies, such as for example, Abnova corporation, Anogen, Alpco Diagnostics, Ray Biotech, alphagenix, autogen, R&D Systems, Pepro Tech EC Ltd, cytolab, Bender MedSystems GmbH, Biovision Research Products, EBD biosciences, Chemicon, Axxora Platform, Promo Cell Distrubuters, Cell Science, Santa Cruz Biotechnology, Sigma etc. can be used. By way of an example only, commercial available antibodies useful in the methods as disclosed herein include, for example CA12, sigma (cat #HPA008773); CA9, US Bio (cat #C1105-80C); EGLN3, US Bio (cat #P3375-06); TGFB3, Cell Sciences (cat #PAAM1); NMU, US Bio (cat #N2171-80H); PMP22, US Bio (cat #P4305-04); TNFRSF7, Sigma (cat #C8974); NYP1, Santa Cruz (Cat #sc-21990) and the like.

In alternative embodiments, antibodies directed against wild type or fragments or variants of RCC biomarker proteins can also be used in disease diagnostics and prognostics. Such diagnostic methods can be used to detect increases in the level of expression of the RCC biomarker protein expression, or abnormalities in the structure and/or tissue, cellular, or subcellular location of the RCC biomarker peptide.

In another embodiment, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of a antibody, wherein the antibodies are used to specifically target molecules inside or on the surface of cells. The antibody typically contains a marker that will undergo a biochemical reaction, and thereby experience a change color, upon encountering the targeted molecules. In some instances, signal amplification can be integrated into the particular protocol, wherein a secondary antibody, that includes the marker stain or marker signal, follows the application of a primary specific antibody.

In some embodiments, the methods as described herein can be performed, for example, by utilizing pre-packaged diagnostic kits, such as those described above, comprising at least one probe which can be conveniently used, e.g., to determine whether a subject has or is at risk of developing disease such as renal cell carcinoma (RCC), in particular clear cell renal cell carcinoma.

The term “protein-binding molecule” or “antibody-based binding moiety” or “antibody” includes immunoglobulin molecules and immunologically active determinants of immunoglobulin molecules, e.g., molecules that contain an antigen binding site which specifically binds (i.e. immunoreacts with) to the Psap proteins. The term “antibody-based binding moiety” is intended to include whole antibodies, e.g., of any isotype (IgG, IgA, IgM, IgE, etc), and includes fragments thereof which are also specifically reactive with the Psap proteins. Antibodies can be fragmented using conventional techniques. Thus, the term includes segments of proteolytically-cleaved or recombinantly-prepared portions of an antibody molecule that are capable of selectively reacting with a certain protein. Non limiting examples of such proteolytic and/or recombinant fragments include Fab, F(ab′)2, Fab′, Fv, dAbs and single chain antibodies (scFv) containing a VL and VH domain joined by a peptide linker. The scFv's can be covalently or non-covalently linked to form antibodies having two or more binding sites. Thus, “antibody-base binding moiety” includes polyclonal, monoclonal, or other purified preparations of antibodies and recombinant antibodies. The term “antibody-base binding moiety” is further intended to include humanized antibodies, bispecific antibodies, and chimeric molecules having at least one antigen binding determinant derived from an antibody molecule. In a preferred embodiment, the antibody-based binding moiety detectably labeled. In some embodiments, a “protein-binding molecule” is a co-factor or binding protein that interacts with the protein to be measured, for example a co-factor or binding protein to a RCC biomarker protein.

Another aspect of the present invention relates to an array comprising a solid platform, including a nanochip or beads (such as disclosed in U.S. patent Application 2007/0065844A1, which is incorporated herein by reference) and protein-binding molecules attached thereto, wherein the array comprises at least 3 and at most 100 different protein-binding molecules in known positions, wherein at least 3 are different protein-protein binding molecules having specific binding affinity for proteins selected from the group the proteins of CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43) or fragments or functional variants or derivatives thereof.

The term “labeled antibody”, as used herein, includes antibodies that are labeled by a detectable means and include, but are not limited to, antibodies that are enzymatically, radioactively, fluorescently, and chemiluminescently labeled. Antibodies can also be labeled with a detectable tag, such as c-Myc, HA, VSV-G, HSV, FLAG, V5, or HIS. The detection and quantification of Psap or Tsp-1 present in the tissue samples correlate to the intensity of the signal emitted from the detectably labeled antibody.

In one embodiment, the antibody-based binding moiety is detectably labeled by linking the antibody to an enzyme. The enzyme, in turn, when exposed to it's substrate, will react with the substrate in such a manner as to produce a chemical moiety which can be detected, for example, by spectrophotometric, fluorometric or by visual means. Enzymes which can be used to detectably label the antibodies of the present invention include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

Detection can also be accomplished using any of a variety of other immunoassays. For example, by radioactively labeling an antibody, it is possible to detect the antibody through the use of radioimmune assays. The radioactive isotope can be detected by such means as the use of a gamma counter or a scintillation counter or by audioradiography. Isotopes which are particularly useful for the purpose of the present invention are ³H, ¹³¹I, ³⁵S, ¹⁴C, and preferably ¹²⁵I.

It is also possible to label an antibody with a fluorescent compound. When the fluorescently labeled antibody is exposed to light of the proper wavelength, its presence can then be detected due to fluorescence. Among the most commonly used fluorescent labeling compounds are CYE dyes, fluorescein isothiocyanate, rhodamine, phycoerytherin, phycocyanin, allophycocyanin, o-phthaldehyde and fluorescamine.

An antibody can also be detectably labeled using fluorescence emitting metals such as 152Eu, or others of the lanthanide series. These metals can be attached to the antibody using such metal chelating groups as diethylenetriaminepentaacetic acid (DTPA) or ethylenediaminetetraacetic acid (EDTA).

An antibody also can be detectably labeled by coupling it to a chemiluminescent compound. The presence of the chemiluminescent-antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

As mentioned above, levels of enzyme protein can be detected by immunoassays, such as enzyme linked immunoabsorbant assay (ELISA), radioimmunoas say (RIA), Immunoradiometric assay (IRMA), Western blotting, immunocytochemistry or immunohistochemistry, each of which are described in more detail below. Immunoassays such as ELISA or RIA, which can be extremely rapid, are more generally preferred. Antibody arrays or protein chips can also be employed, see for example U.S. Patent Application Nos: 20030013208A1; 20020155493A1; 20030017515 and U.S. Pat. Nos. 6,329,209; 6,365,418, which are herein incorporated by reference in their entirety.

Immunoassays

The most common enzyme immunoassay is the “Enzyme-Linked Immunosorbent Assay (ELISA).” ELISA is a technique for detecting and measuring the concentration of an antigen using a labeled (e.g. enzyme linked) form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904.

In a “sandwich ELISA”, an antibody (e.g. anti-enzyme) is linked to a solid phase (i.e. a microtiter plate) and exposed to a biological sample containing antigen (e.g. enzyme). The solid phase is then washed to remove unbound antigen. A labeled antibody (e.g. enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and B-galactosidase. The enzyme linked antibody reacts with a substrate to generate a colored reaction product that can be measured.

In a “competitive ELISA”, antibody is incubated with a sample containing antigen (i.e. enzyme). The antigen-antibody mixture is then contacted with a solid phase (e.g. a microtiter plate) that is coated with antigen (i.e., enzyme). The more antigen present in the sample, the less free antibody that will be available to bind to the solid phase. A labeled (e.g., enzyme linked) secondary antibody is then added to the solid phase to determine the amount of primary antibody bound to the solid phase.

In an “immunohistochemistry assay” a section of tissue is tested for specific proteins by exposing the tissue to antibodies that are specific for the protein that is being assayed. The antibodies are then visualized by any of a number of methods to determine the presence and amount of the protein present. Examples of methods used to visualize antibodies are, for example, through enzymes linked to the antibodies (e.g., luciferase, alkaline phosphatase, horseradish peroxidase, or beta-galactosidase), or chemical methods (e.g., DAB/Substrate chromagen). The sample is then analysed microscopically, most preferably by light microscopy of a sample stained with a stain that is detected in the visible spectrum, using any of a variety of such staining methods and reagents known to those skilled in the art.

Alternatively, “Radioimmunoassays” can be employed. A radioimmunoassay is a technique for detecting and measuring the concentration of an antigen using a labeled (e.g. radioactively or fluorescently labeled) form of the antigen. Examples of radioactive labels for antigens include 3H, 14C, and 125I. The concentration of antigen enzyme in a biological sample is measured by having the antigen in the biological sample compete with the labeled (e.g. radioactively) antigen for binding to an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

In a radioimmunoassay, to determine the concentration of labeled antigen bound to antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with formalin-killed S. aureus. Yet another method for separating the antigen-antibody complex from the free antigen is by performing a “solid-phase radioimmunoas say” where the antibody is linked (e.g., covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the biological sample can be determined.

An “immunoradiometric assay” (IRMA) is an immunoassay in which the antibody reagent is radioactively labeled. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

Other techniques can be used to detect RCC biomarker protein levels in a biological sample can be performed according to a practitioner's preference, and based upon the present disclosure and the type of biological sample (i.e. plasma, urine, tissue sample etc). One such technique is Western blotting (Towbin et al., Proc. Nat. Acad. Sci. 76:4350 (1979)), wherein a suitably treated sample is run on an SDS-PAGE gel before being transferred to a solid support, such as a nitrocellulose filter. Detectably labeled anti-enzyme antibodies can then be used to assess enzyme levels, where the intensity of the signal from the detectable label corresponds to the amount of enzyme present. Levels can be quantified, for example by densitometry.

In one embodiment, RCC biomarkers proteins as disclosed herein, and/or their mRNA levels in the tissue sample can be determined by mass spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. See, e.g., U.S. Pat. No. 5,118,937 (Hillenkamp et al.), and U.S. Pat. No. 5,045,694 (Beavis & Chait).

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094.

Detection of the presence of RCC biomarker mRNA or protein level will typically depend on the detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

### Antibodies or Antisera Against RCC Biomarker Proteins.

In one embodiment, the diagnostic methods as of the present invention uses protein-binding molecules, such as antibodies or anti-sera for determining the expression levels of RCC biomarker proteins, for example antibodies with binding affinities for CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43) or fragments or functional variants or derivatives thereof.

In some embodiments, antibodies useful in the methods and kits of the present invention can be obtained from a commercial source such as, for example but not limited to, anti-CA9, from R&D (cat #AF2188, Rabbit polyclonal-aa59-144); anti-FABP-6 from HyCult Technology (Cat #HP9031, Rabbit polyclonal); anti-TGFb-3 from AbCam (cat#ab15537, Rabbit polyclonal); anti-NMU from Alpha Diagnostics (Cat #NMU61-P, Rabbit polyclonal); anti-PMP22 from AbCam, (cat #ab3278, Mouse monoclonal); anti-PMA2 from AbCam (Cat #ab13705, Rabbit polyclonal) and anti-CD70 from Ancell (Cat #222-020, Mouse monoclonal).

The antibodies can be polyclonal or monoclonal antibodies. Alternatively, antibodies useful in the methods and kits as disclosed herein can be raised against the RCC biomarker proteins, such as the proteins CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43) or fragments or functional variants or derivatives thereof. Methods for the production of enzyme antibodies are disclosed in PCT publication WO 97/40072 or U.S. Application. No. 2002/0182702, which are herein incorporated by reference.

Antibodies for use in the present invention can be produced using standard methods to produce antibodies, for example, by monoclonal antibody production (Campbell, A. M., Monoclonal Antibodies Technology: Laboratory Techniques in Biochemistry and Molecular Biology, Elsevier Science Publishers, Amsterdam, the Netherlands (1984); St. Groth et al., J. Immunology, (1990) 35: 1-21; and Kozbor et al., Immunology Today (1983) 4:72). Antibodies can also be readily obtained by using antigenic portions of the protein to screen an antibody library, such as a phage display library by methods well known in the art. For example, U.S. Pat. No. 5,702,892 (U.S.A. Health & Human Services) and WO 01/18058 (Novopharm Biotech Inc.) disclose bacteriophage display libraries and selection methods for producing antibody binding domain fragments.

By way of examples only, the production of non-human monoclonal antibodies, e.g., murine or rat, can be accomplished by, for example, immunizing the animal with an immunogenic peptide of the a RCC biomarker protein of the present invention, for example but not limited to a protein or fragment thereof selected from the following group: CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43). See Harlow & Lane, Antibodies, A Laboratory Manual (CSHP NY, 1988) (incorporated by reference for all purposes). Such an immunogen can be obtained from a natural source, by peptides synthesis or by recombinant expression.

Humanized forms of mouse antibodies can be generated by linking the CDR regions of non-human antibodies to human constant regions by recombinant DNA techniques. See Queen et al., Proc. Natl. Acad. Sci. USA 86, 10029-10033 (1989) and WO 90/07861 (incorporated by reference for all purposes).

Human antibodies can be obtained using phage-display methods. See, e.g., Dower et al., WO 91/17271; McCafferty et al., WO 92/01047. In these methods, libraries of phage are produced in which members display different antibodies on their outer surfaces. Antibodies are usually displayed as Fv or Fab fragments. Phage displaying antibodies with a desired specificity are selected by affinity enrichment to immunoglobulin lambda 6 light chain or fragments thereof. Human antibodies against immunoglobulin lambda 6 light chain can also be produced from non-human transgenic mammals having transgenes encoding at least a segment of the human immunoglobulin locus and an inactivated endogenous immunoglobulin locus. See, e.g., Lonberg et al., WO93/12227 (1993); Kucherlapati, WO 91/10741 (1991) (each of which is incorporated by reference in its entirety for all purposes). Human antibodies can be selected by competitive binding experiments, or otherwise, to have the same epitope specificity as a particular mouse antibody. Such antibodies are particularly likely to share the useful functional properties of the mouse antibodies. Human polyclonal antibodies can also be provided in the form of serum from humans immunized with an immunogenic agent. Optionally, such polyclonal antibodies can be concentrated by affinity purification using a region of the immunoglobulin lambda light chain, for example a region of the lambda 6 light chain, or other lambda light chain peptides as an affinity reagent.

Human or humanized antibodies can be designed to have IgG, IgD, IgA and IgE constant region, and any isotype, including IgG1, IgG2, IgG3 and IgG4. Antibodies can be expressed as tetramers containing two light and two heavy chains, as separate heavy chains, light chains, as Fab, Fab′F(ab′)₂, and Fv, or as single chain antibodies in which heavy and light chain variable domains are linked through a spacer.

a. Production of Non-Human Antibodies. The production of non-human monoclonal antibodies, e.g., murine, guinea pig, rabbit or rat, can be accomplished by, for example, immunizing the animal with an immunogenic peptides of the present invention, for example but not limited to a peptide with any of SEQ ID NO: 32-43. Any immunogenic peptide substantially similar to a region of the any of the following: CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43) or fragments or functional variants or derivatives thereof are encompassed for use See e.g., Harlow Lane, Antibodies, A Laboratory Manual (CSHP NY, 1988) (incorporated by reference for all purposes). Such immunogenic peptides can be obtained from a natural source, by peptide synthesis or by recombinant expression. Optionally, immunogenic peptides can be administered fused or otherwise complexed with a carrier protein, as described herein. Optionally, immunogenic peptides can be administered with an adjuvant. Several types of adjuvant can be used as described herein. Complete Freund's adjuvant followed by incomplete adjuvant is preferred for immunization of laboratory animals. Rabbits or guinea pigs are typically used for making polyclonal antibodies. Mice are typically used for making monoclonal antibodies. Antibodies are screened for specific binding to the immunogen. Optionally, antibodies are further screened for binding to a specific region of the immunogen, for example the lambda light chain of an immunoglobulin. Binding can be assessed, for example, by Western blot or ELISA. The smallest fragment to show specific binding to the antibody defines the epitope of the antibody. Alternatively, epitope specificity can be determined by a competition assay is which a test and reference antibody compete for binding to the component. If the test and reference antibodies compete, then they bind to the same epitope or epitopes sufficiently proximal that binding of one antibody interferes with binding of the other.\

b. Chimeric and Humanized Antibodies. Chimeric and humanized antibodies have the same or similar binding specificity and affinity as a mouse or other nonhuman antibody that provides the starting material for construction of a chimeric or humanized antibody. Chimeric antibodies are antibodies whose light and heavy chain genes have been constructed, typically by genetic engineering, from immunoglobulin gene segments belonging to different species. For example, the variable (V) segments of the genes from a mouse monoclonal antibody may be joined to human constant (C) segments, such as IgG1 and IgG4. A typical chimeric antibody is thus a hybrid protein consisting of the V or antigen-binding domain from a mouse antibody and the C or effector domain from a human antibody.

Humanized antibodies have variable region framework residues substantially from a human antibody (termed an acceptor antibody) and complementarity determining regions substantially from a mouse-antibody, (referred to as the donor immunoglobulin). See, Queen et al., Proc. Natl. Acad. Sci. USA 86:10029-10033 (1989) and WO 90/07861, U.S. Pat. No. 5,693,762, U.S. Pat. No. 5,693,761, U.S. Pat. No. 5,585,089, U.S. Pat. No. 5,530,101 and Winter, U.S. Pat. No. 5,225,539 (incorporated by reference in their entirety for all purposes). The constant region(s), if present, are also substantially or entirely from a human immunoglobulin. The human variable domains are usually chosen from human antibodies whose framework sequences exhibit a high degree of sequence identity with the murine variable region domains from which the CDRs were derived. The heavy and light chain variable region framework residues can be substantially similar to a region of the same or different human antibody sequences. The human antibody sequences can be the sequences of naturally occurring human antibodies or can be consensus sequences of several human antibodies. See Carter et al., WO 92/22653. Certain amino acids from the human variable region framework residues are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid should usually be substituted by the equivalent framework amino acid from the mouse antibody when it is reasonably expected that the amino acid: (1) noncovalently binds antigen directly, (2) is adjacent to a CDR region, (3) otherwise interacts with a CDR region (e.g. is within about 6 A of a CDR region), or (4) participates in the VL-VH interface.

Other candidates for substitution are acceptor human framework amino acids that are unusual for a human immunoglobulin at that position. These amino acids can be substituted with amino acids from the equivalent position of the mouse donor antibody or from the equivalent positions of more typical human immunoglobulins. Other candidates for substitution are acceptor human framework amino acids that are unusual for a human immunoglobulin at that position. The variable region frameworks of humanized immunoglobulins usually show at least 85% sequence identity to a human variable region framework sequence or consensus of such sequences.

c. Human Antibodies. Human antibodies against Ax3b2 are provided by a variety of techniques described below. Some human antibodies are selected by competitive binding experiments, or otherwise, to have the same epitope specificity as a particular mouse antibody. Human antibodies can also be screened for a particular epitope specificity by using only an immunogenic peptides of the present invention as the immunogen, and/or by screening antibodies for ability to kill plasma cells, as described in the examples.

(1) Trioma Methodology. The basic approach and an exemplary cell fusion partner, SPAZ-4, for use in this approach have been described by Oestberg et al., Hybridoma 2:361-367 (1983); Oestberg, U.S. Pat. No. 4,634,664; and Engleman et al., U.S. Pat. No. 4,634,666 (each of which is incorporated by reference in its entirety for all purposes). The antibody-producing cell lines obtained by this method are called triomas, because they are descended from three cells—two human and one mouse. Initially, a mouse multiple myeloma line is fused with a human B-lymphocyte to obtain a non-antibody-producing xenogeneic hybrid cell, such as the SPAZ-4 cell line described by Oestberg, supra. The xenogeneic cell is then fused with an immunized human B-lymphocyte to obtain an antibody-producing trioma cell line. Triomas have been found to produce antibody more stably than ordinary hybridomas made from human cells.

The immunized B-lymphocytes are obtained from the blood, spleen, lymph nodes or bone marrow of a human donor. If antibodies against a specific antigen or epitope are desired, it is preferable to use that antigen or epitope thereof for immunization. Immunization can be either in vivo or in vitro. For in vivo immunization, B cells are typically isolated from a human immunized with the immunogenic peptides of the present invention, for example proteins or fragments thereof of CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43) or functional variants or derivatives thereof. For in vitro immunization, B-lymphocytes are typically exposed to antigen for a period of 7-14 days in a media such as RPMI-1640 (see Engleman, supra) supplemented with 10% human plasma.

The immunized B-lymphocytes are fused to a xenogeneic hybrid cell such as SPAZ-4 by well known methods. For example, the cells are treated with 40-50% polyethylene glycol of MW 1000-4000, at about 37 degrees C., for about 5-10 min. Cells are separated from the fusion mixture and propagated in media selective for the desired hybrids (e.g., HAT or AH). Clones secreting antibodies having the required binding specificity are identified by assaying the trioma culture medium for the ability to bind to the RCC biomarker proteins as disclosed herein, such as proteins CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43) or fragments or functional variants or derivatives thereof or a fragment thereof. Triomas producing human antibodies having the desired specificity are subcloned by the limiting dilution technique and grown in vitro in culture medium. The trioma cell lines obtained are then tested for the ability to bind to the RCC biomarker that they have affinity for using methods commonly known by one of ordinary skill in the art.

Although triomas are genetically stable they do not produce antibodies at very high levels. Expression levels can be increased by cloning antibody genes from the trioma into one or more expression vectors, and transforming the vector into standard mammalian, bacterial or yeast cell lines, according to methods well known in the art.

(2) Transgenic Non-Human Mammals. Human antibodies against immunoglobulin light chains can also be produced from non-human transgenic mammals having transgenes encoding at least a segment of the human immunoglobulin locus. Usually, the endogenous immunoglobulin locus of such transgenic mammals is functionally inactivated. Preferably, the segment of the human immunoglobulin locus includes unrearranged sequences of heavy and light chain components. Both inactivation of endogenous immunoglobulin genes and introduction of exogenous immunoglobulin genes can be achieved by targeted homologous recombination, or by introduction of YAC chromosomes. The transgenic mammals resulting from this process are capable of functionally rearranging the immunoglobulin component sequences, and expressing a repertoire of antibodies of various isotypes encoded by human immunoglobulin genes, without expressing endogenous immunoglobulin genes. The production and properties of mammals having these properties are described in detail by, e.g., Lonberg et al., WO93/12227 (1993); U.S. Pat. No. 5,877,397, U.S. Pat. No. 5,874,299, U.S. Pat. No. 5,814,318, U.S. Pat. No. 5,789,650, U.S. Pat. No. 5,770,429, U.S. Pat. No. 5,661,016, U.S. Pat. No. 5,633,425, U.S. Pat. No. 5,625,126, U.S. Pat. No. 5,569,825, U.S. Pat. No. 5,545,806, Nature 148, 1547-1553 (1994), Nature Biotechnology 14, 826 (1996), Kucherlapati, WO 91/10741 (1991) (each of which is incorporated by reference in its entirety for all purposes). Transgenic mice are particularly suitable in this regard. Monoclonal antibodies are prepared by, e.g., fusing B-cells from such mammals to suitable multiple myeloma cell lines using conventional Kohler-Milstein technology. Human polyclonal antibodies can also be provided in the form of serum from humans immunized with an immunogenic agent.

(3) Phage Display Methods. A further approach for obtaining anti-immunoglobulin light chains antibodies, for example anti-lambda6 containing immunoglobulin antibodies is to screen a DNA library front human B cells according to the general protocol outlined by Huse et al., Science 246:1275-1281 (1989). For example, as described for trioma methodology, such B cells can be obtained from a human immunized with the immunogenic peptides of the present invention, for example the RCC biomarker proteins or fragments thereof such as proteins CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43) or fragments or functional variants or derivatives thereof. Optionally, such B cells are obtained from a patient who is ultimately to receive antibody treatment. Antibodies binding to an epitope of the RCC biomarker protein are selected. Sequences encoding such antibodies (or binding fragments) are then cloned and amplified. The protocol described by Huse is rendered more efficient in combination with phage-display technology. See, e.g., Dower et al., WO 91/17271 and McCafferty et al., WO 92/01047, U.S. Pat. No. 5,877,218, U.S. Pat. No. 5,871,907, U.S. Pat. No. 5,858,657, U.S. Pat. No. 5,837,242, U.S. Pat. No. 5,733,743 and U.S. Pat. No. 5,565,332 (each of which is incorporated by reference in its entirety for all purposes). In these methods, libraries of phage are produced in which members display different antibodies on their outer surfaces. Antibodies are usually displayed as Fv or Fab fragments. Phage displaying antibodies with a desired specificity are selected by affinity enrichment to proteins; CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43) or fragments or functional variants or derivatives thereof.

In a variation of the phage-display method, human antibodies having the binding specificity of a selected murine antibody can be produced. See Winter, WO 92/20791. In this method, either the heavy or light chain variable region of the selected murine antibody is used as a starting material. If, for example, a light chain variable region is selected as the starting material, a phage library is constructed in which members display the same light chain variable region (i.e., the murine starting material) and a different heavy chain variable region. The heavy chain variable regions are obtained from a library of rearranged human heavy chain variable regions. A phage showing strong specific binding for the component of interest (e.g., at least 10⁸ and preferably at least 10⁹ M⁻¹) is selected. The human heavy chain variable region from this phage then serves as a starting material for constructing a further phage library. In this library, each phage displays the same heavy chain variable region (i.e., the region identified from the first display library) and a different light chain variable region. The light chain variable regions are obtained from a library of rearranged human variable light chain regions. Again, phage showing strong specific binding for amyloid peptide component are selected. These phage display the variable regions of completely human anti-amyloid peptide antibodies. These antibodies usually have the same or similar epitope specificity as the murine starting material.

d. Selection of Constant Region. The heavy and light chain variable regions of chimeric, humanized, or human antibodies can be linked to at least a portion of a human constant region. The choice of constant region depends, in part, whether antibody-dependent complement and/or cellular mediated toxicity is desired. For example, isotopes IgG1 and IgG3 have complement activity and isotypes IgG2 and IgG4 do not. Choice of isotype can also affect passage of antibody into the brain. Light chain constant regions can be lambda or kappa. Antibodies can be expressed as tetramers containing two light and two heavy chains, as separate heavy chains, light chains, as Fab, Fab F(ab)², and Fv, or as single chain antibodies in which heavy and light chain variable domains are linked through a spacer.

e. Expression of Recombinant Antibodies. Chimeric, humanized and human antibodies are typically produced by recombinant expression. Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the cross-reacting antibodies.

These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences.

E. coli is one prokaryotic host particularly useful for cloning the DNA sequences of the present invention. Microbes, such as yeast are also useful for expression. Saccharomyces is a preferred yeast host, with suitable vectors having expression control sequences, an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

Mammalian cells are a preferred host for expressing nucleotide segments encoding immunoglobulins or fragments thereof. See Winnacker, From Genes to Clones, (VCH Publishers, NY, 1987). A number of suitable host cell lines capable of secreting intact heterologous proteins have been developed in the art, and include CHO cell lines, various COS cell lines, HeLa cells, L cells and multiple myeloma cell lines. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters substantially similar to a region of the endogenous genes, cytomegalovirus, SV40, adenovirus, bovine papillomavirus, and the like. See Co et al., J. Immunol. 148:1149 (1992).

Alternatively, antibody coding sequences can be incorporated in transgenes for introduction into the genome of a transgenic animal and subsequent expression in the milk of the transgenic animal (e.g., according to methods described in U.S. Pat. No. 5,741,957, U.S. Pat. No. 5,304,489, U.S. Pat. No. 5,849,992, all incorporated by reference herein in their entireties). Suitable transgenes include coding sequences for light and/or heavy chains in operable linkage with a promoter and enhancer from a mammary gland specific gene, such as casein or beta lactoglobulin.

The vectors containing the DNA segments of interest can be transferred into the host cell by well-known methods, depending on the type of cellular host. For example, calcium chloride transfection is commonly utilized for prokaryotic cells, whereas calcium phosphate treatment, electroporation, lipofection, biolistics or viral-based transfection can be used for other cellular hosts. Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

Once expressed, antibodies can be purified according to standard procedures of the art, including HPLC purification, column chromatography, gel electrophoresis and the like (see generally, Scopes, Protein Purification (Springer-Verlag, NY, 1982)). The antibodies with affinity for a RCC biomarker protein as disclosed herein can be assessed by one of ordinary skill in the art, such as, for example but not limited to, western blot analysis on a purified RCC biomarker protein, or a biological sample comprising a RCC biomarker protein or fragment or variant thereof.

Detection of antibodies with affinity for a RCC biomarker protein can be achieved by direct labeling of the antibodies themselves, with labels including a radioactive label such as ³H, ¹⁴C, ³⁵S, ¹²⁵I, or ¹³¹I, a fluorescent label, a hapten label such as biotin, or an enzyme such as horse radish peroxidase or alkaline phosphatase. Alternatively, unlabeled primary antibody is used in conjunction with labeled secondary antibody, comprising antisera, polyclonal antisera or a monoclonal antibody specific for the primary antibody. In a preferred embodiment, the primary antibody or antisera is unlabeled, the secondary antisera or antibody is conjugated with biotin and enzyme-linked strepavidin is used to produce visible staining for histochemical analysis.

### Uses

In one embodiment, in view of the currently limited options for RCC management, the group of RCC biomarkers or subgroups thereof as disclosed herein is useful for identifying subjects at risk of developing or having RCC. In some embodiments, the group of RCC biomarkers or subgroups thereof as disclosed herein is useful for identifying subjects with poor-prognosis, in particular subjects with localized RCCs that are likely to relapse and metastasize. Accordingly, subject identified with an increased likelihood of RCC can be administered therapy, for example systematic therapy.

In some embodiments, the compositions and methods as disclosed herein can also be used to identify subjects in need of frequent follow-up by a physician or clinician to monitor RCC disease progression. For example, if a subject is identified to have increased risk of developing RCC using the methods and compositions as disclosed herein, the subject can initiate treatment earlier, when the disease may potentially be more sensitive to treatment.

Screening subjects for identifying subjects at risk of developing or having RCC using the group of RCC biomarkers or subgroups thereof as disclosed herein is also useful to identify subjects most suitable or amenable to be enrolled in clinical trial for assessing a therapy for RCC, which will permit more effective subgroup analyses and follow-up studies. Furthermore, the expression of the group of RCC biomarkers as disclosed herein can be monitored in subjects enrolled in a clinical trial to provide a quantitative measure for the therapeutic efficacy of the therapy which is subject to the clinical trial.

### Methods of Treatment

The invention further provides methods of treating subjects identified, using the methods of the present invention, to be at risk of developing or afflicted with RCC, wherein the biological sample obtained from the subject has increased expression of gene transcripts and/or protein of at least 2 RCC biomarkers as listed in Table 1 as compared to the same genes analyzed in a reference sample.

This invention also provides a method for selecting a therapeutic regimen or determining if a certain therapeutic regimen is more appropriate for a subject identified as having RCC or at increased risk of developing RCC as identified by the methods as disclosed herein. For example, an aggressive anti-cancer therapeutic regime can be perused in which a subject identified with RCC, where the subject is administered a therapeutically effective amount of an anti-cancer agent to treat the RCC. In alternative embodiments, a prophylactic anti-cancer therapeutic regimen can be pursued in a subject identified to have increased likelihood of developing RCC, where the subject is administered a prophylactic dose or maintenance dose of an anti-cancer agent to prevent the development of RCC. In alternative embodiments, a subject can be monitored for RCC using the methods and RCC biomarkers as disclosed herein, and if on a first (i.e. initial) testing the subject is identified as having RCC, the subject can be administered an anti-cancer therapy, and on a second (i.e. follow-up testing), the subject is identified as not having RCC or having decreased levels of protein or gene transcript expression of at the same group of RCC biomarkers as analyzed in the first testing, the subject can be administered an anti-cancer therapy at a maintenance dose.

In general, a therapy is considered to “treat” RCC if it provides one or more of the following treatment outcomes: reduce or delay recurrence of the RCC after the initial therapy; increase median survival time or decrease metastases. The method is particularly suited to determining which subjects will be responsive or experience a positive treatment outcome to a chemotherapeutic regimen. In some embodiments, an anti-cancer therapy is, for example but not limited to administration of a chemotherapeutic agents such as fluoropyrimidine drug such as 5-FU or a platinum drug such as oxaliplatin or cisplatin. Alternatively, the chemotherapy includes administration of a topoisomerase inhibitor such as irinotecan. In a yet further embodiment, the therapy comprises administration of an antibody (as broadly defined herein), ligand or small molecule that binds the Epidermal Growth Factor Receptor (EGFR).

In some embodiments, the anti-cancer therapy is a chemotherapeutic agent, radiotherapy etc. Such anti-cancer therapies are disclosed herein, as well as others that are well known by persons of ordinary skill in the art and are encompassed for use in the present invention. In some embodiments the anti-cancer therapy, or cancer prevention strategy is targets the EGF/EGFR pathway, and in other embodiments, the anti-cancer therapy or cancer prevention strategy does not target the EGF/EGFR pathway.

The term “anti-cancer agent” or “anti-cancer drug” is any agent, compound or entity that would be capably of negatively affecting the cancer in the subject, for example killing cancer cells, inducing apoptosis in cancer cells, reducing the growth rate of cancer cells, reducing the number of mestatic cells, reducing tumor size, inhibiting tumor growth, reducing blood supply to a tumor or cancer cells, promoting an immune response against cancer cells or a tumor, preventing or inhibiting the progression of cancer, or increasing the lifespan of the subject with cancer. Anti-cancer therapy includes biological agents (biotherapy), chemotherapy agents, and radiotherapy agents. The combination of chemotherapy with biological therapy is known as biochemotherapy.

Treatment can include prophylaxis, including agents which slow or reduce the risk of RCC in a subject. In other embodiments, the treatments are any means to prevent the proliferation of RCC cancerous cells. In some embodiments, the treatment is an agent which suppresses the EGF-EGFR pathway, for example but not limited to inhibitors and agents of EGFR. Inhibitors of EGFR include, but are not limited to, tyrosine kinase inhibitors such as quinazolines, such as PID 153035, 4-(3-chloroanilino) quinazoline, or CP-358,774, pyridopyrimidines, pyrimidopyrimidines, pyrrolopyrimidines, such as CGP 59326, CGP 60261 and CGP 62706, and pyrazolopyrimidines, 4-(phenylamino)-7H-pyrrolo[2,3-d]pyrimidines (Traxler et al., (1996) J. Med Chem 39:2285-2292), curcumin (diferuloyl methane) (Laxmin arayana, et al., (1995), Carcinogen 16:1741-1745), 4,5-bis(4-fluoroanilino)phthalimide (Buchdunger et al. (1995) Clin. Cancer Res. 1:813-821; Dinney et al. (1997) Clin. Cancer Res. 3:161-168); tyrphostins containing nitrothiophene moieties (Brunton et al. (1996) Anti Cancer Drug Design 11:265-295); the protein kinase inhibitor ZD-1 839 (AstraZeneca); CP-358774 (Pfizer, Inc.); PD-01 83805 (Warner-Lambert), EKB-569 (Torrance et al., Nature Medicine, Vol. 6, No. 9, September 2000, p. 1024), HKI-272 and HKI-357 (Wyeth); or as described in International patent application WO05/018677 (Wyeth); WO99/09016 (American Cyanamid); WO98/43960 (American Cyanamid); WO 98/14451; WO 98/02434; WO97/38983 (Warener Labert); WO99/06378 (Warner Lambert); WO99/06396 (Warner Lambert); WO96/30347 (Pfizer, Inc.); WO96/33978 (Zeneca); WO96/33977 (Zeneca); and WO96/33980 (Zeneca), WO 95/19970; U.S. Pat. App. Nos. 2005/0101618 assigned to Pfizer, 2005/0101617, 20050090500 assigned to OSI Pharmaceuticals, Inc.; all herein incorporated by reference. Further useful EGFR inhibitors are described in U.S. Pat. App. No. 20040127470, particularly in tables 10, 11, and 12, and are herein incorporated by reference.

In another embodiment, the anti-cancer therapy includes a chemotherapeutic regimen further comprises radiation therapy. In an alternate embodiment, the therapy comprises administration of an anti-EGFR antibody or biological equivalent thereof.

In some embodiments, the anti cancer treatment comprises the administration of a chemotherapeutic drug selected from the group consisting of fluoropyrimidine (e.g., 5-FU), oxaliplatin, CPT-11, (e.g., irinotecan) a platinum drug or an anti EGFR antibody, such as the cetuximab antibody or a combination of such therapies, alone or in combination with surgical resection of the tumor. In yet a further aspect, the treatment compresses radiation therapy and/or surgical resection of the tumor masses. In one embodiment, the present invention encompasses administering to a subject identified as having, or increased risk of developing RCC an anti-cancer combination therapy where combinations of anti-cancer agents are used, such as for example Taxol, cyclophosphamide, cisplatin, gancyclovir and the like. Anti-cancer therapies are well known in the art and are encompassed for use in the methods of the present invention. Chemotherapy includes, but is not limited to an alkylating agent, mitotic inhibitor, antibiotic, or antimetabolite, anti-angliogenic agents etc. The chemotherapy can comprise administration of CPT-11, temozolomide, or a platin compound. Radiotherapy can include, for example, x-ray irradiation, w-irradiation, γ-irradiation, or microwaves.

The term “chemotherapeutic agent” or “chemotherapy agent” are used interchangeably herein and refers to an agent that can be used in the treatment of cancers and neoplasms, for example brain cancers and gliomas and that is capable of treating such a disorder. In some embodiments, a chemotherapeutic agent can be in the form of a prodrug which can be activated to a cytotoxic form. Chemotherapeutic agents are commonly known by persons of ordinary skill in the art and are encompassed for use in the present invention. For example, chemotherapeutic drugs for the treatment of tumors and gliomas include, but are not limited to: temozolomide (Temodar), procarbazine (Matulane), and lomustine (CCNU). Chemotherapy given intravenously (by IV, via needle inserted into a vein) includes vincristine (Oncovin or Vincasar PFS), cisplatin (Platinol), carmustine (BCNU, BiCNU), and carboplatin (Paraplatin), Mexotrexate (Rheumatrex or Trexall), irinotecan (CPT-11); erlotinib; oxalipatin; anthracyclins-idarubicin and daunorubicin; doxorubicin; alkylating agents such as melphalan and chlorambucil; cis-platinum, methotrexate, and alkaloids such as vindesine and vinblastine.

In another embodiment, the present invention encompasses combination therapy in which subjects identified as having, or increased risk of developing RCC using the methods as disclosed herein are administered an anti-cancer combination therapy where combinations of anti-cancer agents are used are used in combination with cytostatic agents, anti-VEGF and/or p53 reactivation agent. A cytostatic agent is any agent capable of inhibiting or suppressing cellular growth and multiplication. Examples of cytostatic agents used in the treatment of cancer are paclitaxel, 5-fluorouracil, 5-fluorouridine, mitomycin-C, doxorubicin, and zotarolimus. Other cancer therapeutics include inhibitors of matrix metalloproteinases such as marimastat, growth factor antagonists, signal transduction inhibitors and protein kinase C inhibitors.

Some examples of anti-VEGF agents include bevacizumab (Avastin™), VEGF Trap, CP-547,632, AG13736, AG28262, SU5416, SU11248, SU6668, ZD-6474, ZD4190, CEP-7055, PKC 412, AEE788, AZD-2171, sorafenib, vatalanib, pegaptanib octasodium, IM862, DC101, angiozyme, Sirna-027, caplostatin, neovastat, ranibizumab, thalidomide, and AGA-1470, a synthetic analog of fumagillin (alternate names: Amebacilin, Fugillin, Fumadil B, Fumadil) (A. G. Scientific, catalog #F1028), an angio-inhibitory compound secreted by Aspergillus fumigates.

As used herein the term “anti-VEGF agent” refers to any compound or agent that produces a direct effect on the signaling pathways that promote growth, proliferation and survival of a cell by inhibiting the function of the VEGF protein, including inhibiting the function of VEGF receptor proteins. The term “agent” or “compound” as used herein means any organic or inorganic molecule, including modified and unmodified nucleic acids such as antisense nucleic acids, RNAi agents such as siRNA or shRNA, peptides, peptidomimetics, receptors, ligands, and antibodies. Preferred VEGF inhibitors, include for example, AVASTIN® (bevacizumab), an anti-VEGF monoclonal antibody of Genentech, Inc. of South San Francisco, Calif., VEGF Trap (Regeneron/Aventis). Additional VEGF inhibitors include CP-547,632 (3-(4-Bromo-2,6-difluoro-benzyloxy)-5-[3-(4-pyrrolidin 1-yl-butyl)-ureido]-isothiazole-4-carboxylic acid amide hydrochloride; Pfizer Inc., NY), AG13736, AG28262 (Pfizer Inc.), SU5416, SU11248, & SU6668 (formerly Sugen Inc., now Pfizer, New York, N.Y.), ZD-6474 (AstraZeneca), ZD4190 which inhibits VEGF-R2 and -R1 (AstraZeneca), CEP-7055 (Cephalon Inc., Frazer, Pa.), PKC 412 (Novartis), AEE788 (Novartis), AZD-2171), NEXAVAR® (BAY 43-9006, sorafenib; Bayer Pharmaceuticals and Onyx Pharmaceuticals), vatalanib (also known as PTK-787, ZK-222584: Novartis & Schering: AG), MACUGEN® (pegaptanib octasodium, NX-1838, EYE-001, Pfizer Inc./Gilead/Eyetech), IM862 (glufanide disodium, Cytran Inc. of Kirkland, Wash., USA), VEGFR2-selective monoclonal antibody DC101 (ImClone Systems, Inc.), angiozyme, a synthetic ribozyme from Ribozyme (Boulder, Colo.) and Chiron (Emeryville, Calif.), Sirna-027 (an siRNA-based VEGFR1 inhibitor, Sirna Therapeutics, San Francisco, Calif.) Caplostatin, soluble ectodomains of the VEGF receptors, Neovastat (AEterna Zentaris Inc; Quebec City, Calif.) and combinations thereof.

The compounds used in connection with the treatment methods of the present invention are administered and dosed in accordance with good medical practice, taking into account the clinical condition of the individual subject, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including, but not limited to, improved survival rate or more rapid recovery, or improvement or elimination of symptoms and other indicators as are selected as appropriate measures by those skilled in the art.

The methods of the present invention are useful for the early detection of subjects susceptible to developing RCC. Thus, treatment may be initiated early, e.g. before or at the beginning of the onset of symptoms, for example before the onset of RCC. In alternative embodiments, the treatment may be administered to a subject that has, or is at risk of developing RCC. In alternative embodiments, the treatment may be administered prior to, during, concurrent or post the development of RCC. The effective amount or dosage required at these early stages will typically be lower than those needed at later stages of disease where the symptoms of RCC are severe. Such dosages are known to those of skill in the art and can be determined by a physician.

In some embodiments, where a subject is identified as having increased risk of having or developing RCC using the RCC biomarkers and methods as disclosed herein, a clinician can recommended a treatment regimen to reduce or lower the expression levels of the RCC biomarkers in the subject. Accordingly, the methods of the present invention provide preventative methods to reduce the risk of a subject getting RCC by reducing the protein and/or gene transcript levels of at least 2 of the RCC biomarkers as listed in Table 1, but preferably by reducing the protein and/or gene transcript levels of about 3, about 4, about 4, about 5, about 6, about 7, about 8, about 9, about 10, about 11 RCC biomarkers as listed in Table 1 in the subject.

In another embodiment, a subject with identified as having or at risk of developing RCC using the methods as disclosed herein can be monitored for levels of the proteins and/or gene transcripts encoded by the RCC biomarkers in a biological sample before, during and after a anti-cancer therapy or treatment regimen, and where a subject is identified to not have lowered the expression levels of the proteins and/or gene transcripts of at least 2 RCC biomarkers, (and thus is still is at risk of having or developing RCC) after a period of time of being administered such a treatment regimen, then the treatment regimen could be modified, for example the subject could be administered (i) a different anti-cancer therapy or anti-cancer drug (ii) a different amount such as in increased amount or dose of a anti-cancer therapy or anti-cancer drug or (iii) a combination of anti-cancer therapies etc.

### Kits of the Present Invention

In some embodiments, the present invention provides diagnostic methods for determining the likelihood of a subject having or developing RCC by gene expression analysis of at least 2 or at least 3 gene transcripts of the RCC biomarkers as listed in Table 1. In some embodiments, the methods use probes or primers comprising nucleotide sequences which are complementary to the genes in the group of RCC biomarkers, or subgroup thereof in any combination. Accordingly, the invention provides kits for performing these methods.

The kit can comprise at least two probes or two primer-pairs which are capable of specifically hybridizing to at least two genes selected from the group of RCC biomarkers as disclosed in Table 1 and instructions for use. Preferred kits amplify a portion of at least 2 gene transcripts, or at least 3-5, or about 5-7, or about 7-9 or about 9-11 gene transcripts selected from the group of RCC biomarkers as disclosed in Table 1. Such kits are suitable for detection of level of transcript expression by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the preferred surface is silica or glass. In another embodiment, the surface is a metal electrode.

An array comprising polynucleotide binding probes or protein-binding molecules to the RCC biomarkers as disclosed herein are useful in the methods as disclosed herein. An array can be made of any conventional substrate. Moreover, the array can be in any shape that can be read, including rectangular and spheroid. Preferred substrates are any suitable rigid or semi-rigid support including membranes, filter, chips, slides, wafers, fibers, magnetic or nonmagnetic beads, gels, tubing, plates, polymers, microparticles and capillaries. The substrate can have a variety of surface forms, such as wells, trenches, pins, channels and pores, to which the peptides and/or antibodies are bound. Preferably, the substrates are optically transparent. Any type of substrate will be a suitable “chip” as long as the probes, such as antibodies can be used as bait to fish for expressed RCC biomarker proteins in a biological sample.

The term “antibody array” refers to an ordered arrangement of antibodies, that specifically bind to peptide microarrays, on a substrate such as a glass, nylon, or a bead, such as SPA beads which is based on either yttrium silicate (YSi) which has scintillant properties by virtue of cerium ions within the crystal lattice, or polyvinyltoluene (PVT) which acts as a solid solvent for anthrancine (DPA) (Amersham Biosciences, Piscataway, N.J.).

The antibodies are arranged on the flat or spherical substrate referred hereto as a “chip” so that there are preferably at least one or more different antibodies, more preferably at least about 50 antibodies, still more preferably at least about 100 antibodies, and most preferably at least about 1,000 antibodies, on a 1 cm.sup.2 substrate surface. The maximum number of antibodies on a substrate is unlimited, but can be at least about 100,000 antibodies.

The term “peptide microarray” refers to a microarray of peptides, wherein one or more of the peptides are from a coding region of the genome. Preferably, the peptides cover at least the coding regions that are of interest and contain an antigenic epitope. More preferably the peptide has an epitope that approximates the wild type conformation of the protein of interest.

Such antibody arrays can be used to screen a biological sample of interest. The proteins in the sample that bind to the array can be readily determined by a range of known means based upon this disclosure. For example, the target proteins and the antibodies may be labeled with one or more labeling moieties to allow detection of both protein-antibody complexes and by comparison the lack of such a complex in the comparison sample. The labeling moieties can include compositions that can be detected by photochemical, spectroscopic, biochemical, immunochemical, chemical, optical, electrical, bioelectronic, etc. means. Labeling moieties include chemiluminescent compounds, radioisotopes, labeled compounds, spectroscopic markers such as fluorescent molecules, magnetic labels, mass spectrometry tags, electron transfer donors and/or acceptors, etc.

Yet other kits of the invention comprise at least one reagent necessary to perform the assay. For example, the kit can comprise an enzyme. Alternatively the kit can comprise a buffer or any other necessary reagent.

Conditions for incubating a nucleic acid probe with a biological sample depend on the format employed in the assay, the detection methods used, and the type and nature of the nucleic acid probe used in the assay. One skilled in the art will recognize that any one of the commonly available hybridization, amplification or immunological assay formats can readily be adapted to employ the nucleic acid probes for use in the present invention.

In alternative embodiments, the present invention provides diagnostic methods for determining the likelihood of a subject having or developing RCC by protein expression analysis of at least 2 or at least 3 proteins encoded by the RCC biomarkers as listed in Table 1.

In some embodiments, the biological samples used in the diagnostic kits include cells, protein or membrane extracts of cells, or biological fluids such as sputum, blood, serum, plasma, or urine. The biological sample used in the above described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the reference biological samples as well as positive and negative controls, reagents, primers, sequencing markers, probes and antibodies described herein for determining the protein and/or gene transcript expression level of at least 2 or at least 3 RCC biomarkers as disclosed herein, in order to determine a subject's likelihood of having or being at risk of developing RCC.

As amenable, these suggested kit components may be packaged in a manner customary for use by those of skill in the art. For example, these suggested kit components may be provided in solution or as a liquid dispersion or the like.

The invention also provides diagnostic and experimental kits which include antibodies for determining the protein expression level encoded by at least 2 or at least 3 RCC biomarkers as disclosed herein, in order to determine a subject's likelihood of having or being at risk of developing RCC. In such kits, the antibodies may be provided with means for binding to detectable marker moieties or substrate surfaces. Alternatively, the kits may include the antibodies already bound to marker moieties or substrates. The kits may further include reference biological samples as well as positive and/or negative control reagents as well as other reagents for adapting the use of the antibodies to particular experimental and/or diagnostic techniques as desired. The kits may be prepared for in vivo or in vitro use, and may be particularly adapted for performance of any of the methods of the invention, such as ELISA. For example, kits containing antibody bound to multi-well microtiter plates can be manufactured.

Other objects, features and advantages will become apparent from the following detailed description. It should be understood, however, that the detailed description and specific examples, while indicating specific embodiments of the invention, are given by way of illustration only, since various changes and modifications within the spirit and scope if the invention will become apparent to those skilled in the art from this detailed description.

The invention now being generally described, it will be more readily understood by reference to the following examples which are included merely for purposes of illustration of certain aspects and embodiments of the present invention, and are not intended to limit the invention

The following examples are provided to illustrate certain embodiments of the invention. They are not intended to limit in any way the remainder of the disclosure.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs. Practitioners are particularly directed to Sambrook, et al. (1989) Molecular Cloning: A Laboratory Manual (Second Edition), Cold Spring Harbor Press, Plainview, N.Y. and Ausubel, F. M., et al. (1998) Current Protocols in Molecular Biology, John Wiley Sons, New York, N.Y., for definitions, terms of art and standard methods known in the art of biochemistry and molecular biology. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used in this invention. Although any methods and materials similar or equivalent to those described herein can be used in the practice or testing of the present invention, the preferred methods and materials are described. Numeric ranges are inclusive of the numbers defining the range. Unless otherwise indicated, nucleic acids are written left to right in 5′ to 3′ orientation; amino acid sequences are written left to right in amino to carboxy orientation, respectively. The headings provided herein are not limitations of the various aspects or embodiments of the invention which can be had by reference to the specification as a whole. Accordingly, the terms defined immediately below are more fully defined by reference to the specification as a whole.

It is understood that this invention is not limited to the particular methodology, protocols, and reagents described, as these may be varied to produce the same result.

The term “gene” used herein refers to a nucleic acid sequence encoding an amino acid sequence or a functional RNA, such as mRNA, tRNA, rRNA, catalytic RNA, siRNA, miRNA and antisense RNA. A gene can also be an mRNA or cDNA corresponding to the coding regions (e.g. exons and miRNA). A gene can also be an amplified nucleic acid molecule produced in vitro comprising all or a part of the coding region.

The term “gene product” as used herein refers to both an RNA transcript of a gene and a translated polypeptide encoded by that transcript.

The term “expression” as used herein refers to transcription of a nucleic acid sequence, as well as to the production, by translation, of a polypeptide product from a transcribed nucleic acid sequence.

The term “nucleic acid” or “oligonucleotide” or “polynucleotide” used herein can mean at least two nucleotides covalently linked together. As will be appreciated by those skilled in the art, the depiction of a single strand also defines the sequence of the complementary strand. Thus, a nucleic acid also encompasses the complementary strand of a depicted single strand. As will also be appreciated by those in the art, many variants of a nucleic acid can be used for the same purpose as a given nucleic acid. Thus, a nucleic acid also encompasses substantially identical nucleic acids and complements thereof. As will also be appreciated by those in the art, a single strand provides a probe that can hybridize to a target sequence under stringent hybridization conditions. Thus, a nucleic acid also encompasses a probe that hybridizes under stringent hybridization conditions.

The term “expression” as used herein refers to interchangeably to the expression of a polypeptide or protein or expression of a polynucleotide or expression of a gene. Expression also refers to the expression of pre-translational modified and post-translationally modified proteins, as well as expression of pre-mRNA molecules, alternatively spliced and mature mRNA molecules. Expression of a polynucleotide can be determined, for example, by measuring the production of RNA transcript molecules, for example messenger RNA (mRNA) transcript levels. Expression of a protein or polypeptide can be determined, for example, by immunoassay using an antibody(ies) that bind with the polypeptide.

The term “encode” as it is applied to polynucleotides refers to a polynucleotide which is said to “encode” a polypeptide or protein if, in its native state or when manipulated by methods well known to those skilled in the art, it can be transcribed to produce the RNA which can be translated into an amino acid sequence to generate the polypeptide and/or a fragment thereof. The antisense strand is the complement of such a nucleic acid, and the encoding sequence can be deduced therefrom.

The terms “polypeptide” and “protein” are used interchangeably herein and include a molecular chain of amino acids linked through peptide bonds. The terms do not refer to a specific length of the product. Thus, “peptides,” “oligopeptides,” and “proteins” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide.

The terms “protein-binding molecule” refers to a agent or protein which specifically binds to an protein, such as an a protein-binding molecule which specifically binds a RCC biomarker protein. Protein-binding molecules are well known in the art, and include antibodies, protein-binding peptide and the like. The region on the protein which binds to the protein-binding molecule is referred to as the epitope, and the protein which is bound to the protein-binding molecule is often referred to in the art as an antigen.

The terms “specifically binds,” “specific binding affinity” (or simply “specific affinity”), and “specifically recognize,” and other related terms when used to refer to binding between a protein and an antibody, refers to a binding reaction that is determinative of the presence of the protein in the presence of a heterogeneous population of proteins and other biologics. Thus, under designated conditions, a specified antibody binds preferentially to a particular protein and does not bind in a significant amount to other proteins present in the sample. An antibody that specifically binds to a protein has an association constant of at least 10³ M⁻¹ or 10⁴ M⁻¹, sometimes 10⁵ M⁻¹ or 10⁶ M⁻¹, in other instances 10⁶M⁻¹ or 10⁷ M⁻¹, preferably 10⁸ M⁻¹ to 10⁹ M⁻¹, and more preferably, about 10¹⁰ M⁻¹ to 10¹¹ M⁻¹ or higher. Protein-binding molecules with affinities greater than 10⁸ M⁻¹ are useful in the methods of the present invention. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

The term “variant” as used herein refers to a peptide or nucleic acid that differs from the naturally occurring polypeptide or nucleic acid by one or more amino acid or nucleic acid deletions, additions, substitutions or side-chain modifications, yet retains one or more specific functions or biological activities of the naturally occurring molecule. Amino acid substitutions include alterations in which an amino acid is replaced with a different naturally-occurring or a non-conventional amino acid residue. Such substitutions may be classified as “conservative”, in which case an amino acid residue contained in a polypeptide is replaced with another naturally occurring amino acid of similar character either in relation to polarity, side chain functionality or size. Substitutions encompassed by the present invention may also be “non conservative”, in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments amino acid substitutions are conservative. Also encompassed within the term variant when used with reference to a polynucleotide or polypeptide, refers to a polynucleotide or polypeptide that can vary in primary, secondary, or tertiary structure, as compared to a reference polynucleotide or polypeptide, respectively (e.g., as compared to a wild-type polynucleotide or polypeptide). A “variant” of a RCC biomarker polypeptide, for example SEQ ID NOs: 32-43 is meant to refer to a molecule substantially similar in structure and function to the proteins of SEQ ID NOs: 32-43 respectively.

For example, a variant of an RCC biomarker peptide can contain a mutation or modification that differs from a reference amino acid in SEQ ID NOs: 32-43. In some embodiments, a variant of SEQ ID NOs: 32-43 is a fragment of SEQ ID NOs: 32-43 as disclosed herein. In some embodiments, a variant can be a different isoform of SEQ ID NOs: 32-43 or can comprise different isomer amino acids. Variants can be naturally-occurring, synthetic, recombinant, or chemically modified polynucleotides or polypeptides isolated or generated using methods well known in the art. Variants can include conservative or non-conservative amino acid changes, as described below. Polynucleotide changes can result in amino acid substitutions, additions, deletions, fusions and truncations in the polypeptide encoded by the reference sequence. Variants can also include insertions, deletions or substitutions of amino acids, including insertions and substitutions of amino acids and other molecules) that do not normally occur in the peptide sequence that is the basis of the variant, for example but not limited to insertion of ornithine which do not normally occur in human proteins. The term “conservative substitution,” when describing a polypeptide, refers to a change in the amino acid composition of the polypeptide that does not substantially alter the polypeptide's activity. For example, a conservative substitution refers to substituting an amino acid residue for a different amino acid residue that has similar chemical properties. Conservative amino acid substitutions include replacement of a leucine with an isoleucine or valine, an aspartate with a glutamate, or a threonine with a serine. “Conservative amino acid substitutions” result from replacing one amino acid with another having similar structural and/or chemical properties, such as the replacement of a leucine with an isoleucine or valine, an aspartate with a glutamate, or a threonine with a serine. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not reduce the activity of the peptide, (i.e. the ability of the peptide to penetrate the BBB). Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984).) In some embodiments, individual substitutions, deletions or additions that alter, add or delete a single amino acid or a small percentage of amino acids can also be considered “conservative substitutions” is the change does not reduce the activity of the peptide (i.e. the function of the proteins of SEQ ID NOs: 32-43). Insertions or deletions are typically in the range of about 1 to 5 amino acids, but can include more than 5 amino acids. The choice of conservative amino acids may be selected based on the location of the amino acid to be substituted in the peptide, for example if the amino acid is on the exterior of the peptide and expose to solvents, or on the interior and not exposed to solvents.

In alternative embodiments, one can select the amino acid which will substitute an existing amino acid based on the location of the existing amino acid, i.e. its exposure to solvents (i.e. if the amino acid is exposed to solvents or is present on the outer surface of the peptide or polypeptide as compared to internally localized amino acids not exposed to solvents). Selection of such conservative amino acid substitutions are well known in the art, for example as disclosed in Dordo et al, J. Mol. Biol, 1999, 217, 721-739 and Taylor et al, J. Theor. Biol. 119 (1986); 205-218 and S. French and B. Robson, J. Mol. Evol. 19 (1983)171. Accordingly, one can select conservative amino acid substitutions suitable for amino acids on the exterior of a protein or peptide (i.e. amino acids exposed to a solvent), for example, but not limited to, the following substitutions can be used: substitution of Y with F, T with S or K, P with A, E with D or Q, N with D or G, R with K, G with N or A, T with S or K, D with N or E, I with L or V, F with Y, S with T or A, R with K, G with N or A, K with R, A with S, K or P.

In alternative embodiments, one can also select conservative amino acid substitutions encompassed suitable for amino acids on the interior of a protein or peptide, for example one can use suitable conservative substitutions for amino acids is on the interior of a protein or peptide (i.e. the amino acids are not exposed to a solvent), for example but not limited to, one can use the following conservative substitutions: where Y is substituted with F, T with A or S, I with L or V, W with Y, M with L, N with D, G with A, T with A or S, D with N, I with L or V, F with Y or L, S with A or T and A with S, G, T or V. In some embodiments, non-conservative amino acid substitutions are also encompassed within the term of variants. A variant of a RCC biomarker polypeptide, for example a variant of SEQ ID NOs: 32-43 is meant to refer to any molecule substantially similar in structure and function to either the entire molecule of SEQ ID NOs: 32-43, or to a fragment thereof.

The term “derivative” as used herein refers to peptides which have been chemically modified, for example but not limited to by techniques such as ubiquitination, labeling, pegylation (derivatization with polyethylene glycol) or addition of other molecules. A molecule also a “derivative” of another molecule when it contains additional chemical moieties not normally a part of the molecule. Such moieties can improve the molecule's solubility, absorption, biological half life, etc. The moieties can alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., MackPubl., Easton, Pa. (1990).

The term “functional” when used in conjunction with “fragment” “derivative” or “variant” refers to a molecule such as a protein which possess a biological activity (either functional or structural) that is substantially similar to a biological activity of the entity or molecule its is a functional derivative or functional variant thereof. The term functional derivative is intended to include the fragments, analogues or chemical derivatives of a molecule.

A molecule is said to be “substantially similar” to another molecule if both molecules have substantially similar structures or if both molecules possess a similar biological activity. Thus, provided that two molecules possess a similar activity, (i.e. a variant of a RCC biomarker protein and the RCC biomarker polypeptide) are considered variants and are encompassed for use as disclosed herein, even if the structure of one of the molecules not found in the other, or if the sequence of amino acid residues is not identical. Thus, provided that two molecules possess a similar biological activity, they are considered variants as that term is used herein even if the structure of one of the molecules not found in the other, or if the sequence of amino acid residues is not identical.

As used herein, the term “nonconservative” refers to substituting an amino acid residue for a different amino acid residue that has different chemical properties. The nonconservative substitutions include, but are not limited to aspartic acid (D) being replaced with glycine (G); asparagine (N) being replaced with lysine (K); or alanine (A) being replaced with arginine (R).

The term “insertions” or “deletions” are typically in the range of about 1 to 5 amino acids. The variation allowed can be experimentally determined by producing the peptide synthetically while systematically making insertions, deletions, or substitutions of nucleotides in the sequence using recombinant DNA techniques.

The term “substitution” when referring to a peptide, refers to a change in an amino acid for a different entity, for example another amino acid or amino-acid moiety. Substitutions can be conservative or non-conservative substitutions.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarily determining region (CDR) of a heavy or light chain or a ligand binding portion thereof, a heavy chain or light chain variable region, a heavy chain or light chain constant region, a framework (FR) region, or any portion thereof, or at least one portion of a binding protein, any of which can be incorporated into an antibody of the present invention. The antibodies can be polyclonal or monoclonal and can be isolated from any suitable biological source, e.g., murine, rat, sheep and canine. Additional sources are identified infra. The term “antibody” is further intended to encompass digestion fragments, specified portions, derivatives and variants thereof, including antibody mimetics or comprising portions of antibodies that mimic the; structure and/or function of an antibody or specified fragment or portion thereof, including single chain antibodies and fragments thereof. Examples of binding fragments encompassed within the term “antigen binding portion” of an antibody include a Fab fragment, a monovalent fragment consisting of the VL, VH, CL and CH, domains; a F(ab′) 2 fragment, a bivalent fragment comprising two Fab fragments linked by a disulfide bridge at the hinge region; a Ed fragment consisting of the VH and CH, domains; a Fv fragment consisting of the VL and VH domains of a single arm of an antibody, a dAb fragment (Ward et al. (1989) Nature 341:544-546), which consists of a VH domain; and an isolated complementarily determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)). Bird et al. (1988) Science 242:423-426 and Huston et al. (1988) Proc. Natl. Acad. Sci. USA 85:5879-5883. Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. The phrase can also refer to continuous or discontinuous epitopes in which the primary sequence (i.e., the amino acid sequence) is not similar but nonetheless the epitopes are still recognized by the same antibody.

The term “antibody variant” is intended to include antibodies produced in a species other than a mouse. It also includes antibodies containing post translational modifications to the linear polypeptide sequence of the antibody or fragment. It further encompasses fully human antibodies. The term “antibody derivative” is intended to encompass molecules that bind an epitope as defined above and which are modifications or derivatives of a native monoclonal antibody of this invention. Derivatives include, but are not limited to, for example, bispecific, multispecific, heterospecific, trispecific, tetraspecific, multispecific antibodies, diabodies, chimeric, recombinant and humanized.

The term “bispecific molecule” is intended to include any agent, e.g., a protein, peptide, or protein or peptide complex, which has two different binding specificities. The term “multispecific molecule” or “heterospecific molecule” is intended to include any agent, e.g. a protein, peptide, or protein or peptide complex, which has more than two different binding specificities.

The term “heteroantibodies” refers to two or more antibodies, antibody binding fragments (e.g., Fab), derivatives thereof, or antigen binding regions linked together, at least two of which have different specificities.

The term “human antibody” as used herein, is intended to include antibodies having variable and constant regions derived from human germline immunoglobulin sequences. The human antibodies of the present invention can include amino acid residues not encoded by human germline immunoglobulin sequences (e.g., mutations introduced by random or site-specific mutagenesis in vitro or by somatic mutation in viva). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, CL, CH domains (e.g., CH1, CH2, CH3), hinge, (Via, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain); genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody can be at least about 95%, or even at least about 96%, or least about 97%, or least about 98%, or least about 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody can display no more than 5, or even no more than 4, 3, 2, or 1 amino acid difference from the amino acid sequence encoded by the germline immunoglobulin gene.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human monoclonal antibody” refers to antibodies displaying a single binding specificity which have variable and constant regions derived from human germline immunoglobulin sequences. The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in viva somatic mutagenesis) and thus the amino acid sequences of the VH and VL regions of the recombinant antibodies are sequences that, while derived from and related to human germline VH and VL sequences, can not naturally exist within the human antibody germline repertoire in vivo. As used herein, “isotype” refers to the antibody class (e.g., IgM or IgG1) that is encoded by heavy chain constant region genes.

An “antigen-binding site” or “binding portion” refers to the part of an immunoglobulin molecule that participates in antigen binding. The antigen-binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions” or “FRs”. Thus, the term “FR” refers to amino acid sequences that are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen binding “surface”. This surface mediates recognition and binding of the target antigen. The three hypervariable regions of each of the heavy and light chains are referred to as “complementarity determining regions” or “CDRs” and are characterized, for example by Kabat et al. Sequences of proteins of immunological interest, 4th ed. U.S. Dept. Health and Human Services, Public Health Services, Bethesda, Md. (1987).

An “array” broadly refers to an arrangement of agents (e.g., proteins, antibodies, replicable genetic packages) in positionally distinct locations on a substrate. In some instances the agents on the array are spatially encoded such that the identity of an agent can be determined from its location on the array. A “microarray” generally refers to an array in which detection requires the use of microscopic detection to detect complexes formed with agents on the substrate. A “location” on an array refers to a localized area on the array surface that includes agents, each defined so that it can be distinguished from adjacent locations (e.g., being positioned on the overall array, or having some detectable characteristic, that allows the location to be distinguished from other locations). Typically, each location includes a single type of agent but this is not required. The location can have any convenient shape (e.g., circular, rectangular, elliptical or wedge-shaped). The size or area of a location can vary significantly. In some instances, the area of a location is greater than 1 cm2, such as 2 cm2, including any area within this range. More typically, the area of the location is less than 1 cm2, in other instances less than 1 mm2, in still other instances less than 0.5 mm2, in yet still other instances less than 10,000 □.m2, or less than 100 □.m2.

A “label” refers to an agent that can be detected by using physical, chemical, optical, electromagnetic and/or other methods. Examples of detectable labels that can be utilized include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

The term “endogenously expressed” or “endogenous expression” refers to the expression of a gene product at normal levels and under normal regulation for that cell type.

The terms “oligonucleotide” or “polynucleotide”, or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and can be chemically or biochemically modified or can contain! non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.); pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. The term “oligonucleotide” as used herein includes a polynucleotide molecule comprising any number of nucleotides which has sufficient number of bases to be used as an oligomer, aptimer or probe in a polymerase chain reaction (PCR). Oligonucleotides are prepared from genomic or cDNA sequence and used to amplify, reveal and confirm the presence of similar DNA or RNA in a particular cell or tissue. Oligonucleotides or oligomers comprise portions of a DNA sequence having at least about 10 nucleotides and as many as about 35 nucleotides and preferably, less than about 200 nucleotides. Oligonucleotides can be between about 5 and about 100 nucleotides in length, preferably between at least about 10 to about 50 nucleotides in length. The exact length of a particular oligonucleotide, however, will depend on many factors, which in turn depend on the ultimate function or use of the oligonucleotide. Oligonucleotides can be synthesized chemically by any suitable means known in the art or derived from a biological sample, as for example, by restriction digestion. The source of the oligonucleotides is not essential to the present invention. Oligonucleotides can be labeled, according to any technique known in the art, such as with radiolabels, fluorescent labels, enzymatic labels, proteins, haptens, antibodies, sequence tags, mass tags, fluorescent polarization etc.

The term “real-time quantitative RT-PCR” or “quantitative RT-PCR” or “QRT-PCR” are used interchangeably herein, refers to reverse transcription (RT) polymerase chain reaction (PCR) which enables detection of gene transcription. The method is known to those ordinary skilled in the art and comprises of the reverse transcription and amplification of messenger RNA (mRNA) species to cDNA, which is further amplified by the PCR reaction. QRT-PCR enables a one skilled in the art to quantitatively measure the level of gene transcription from the test gene in a particular biological sample. The methods of RNA isolation, RNA reverse transcription (RT) to cDNA (copy DNA) and cDNA or nucleic acid amplification and analysis are routine for one skilled in the art and examples of protocols can be found, for example, in the Molecular Cloning: A Laboratory Manual (3-Volume Set) Ed. Joseph Sambrook, David W. Russel, and Joe Sambrook, Cold Spring Harbor Laboratory; 3rd edition (Jan. 15, 2001), ISBN: 0879695773. Particularly useful protocol source for methods used in PCR amplification is PCR (Basics: From Background to Bench) by M. J. McPherson, S. G. Møller, R. Beynon, C. Howe, Springer Verlag; 1st edition (Oct. 15, 2000), ISBN: 0387916008.

The term “multiplex” as used herein refers to the testing and/or the assessment of more than one gene within the same reaction sample.

The term “amplify” is used in the broad sense to mean creating an amplification product which can include, for example, additional target molecules, or target-like molecules or molecules complementary to the target molecule, which molecules are created by virtue of the presence of the target molecule in the sample. In the situation where the target is a nucleic acid, an amplification product can be made enzymatically with DNA or RNA polymerases or reverse transcriptases. The term “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu, D. Y. et al. (1989) Genomics 4:560-569 (for LCR).

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, interdisposition, affection. A disease and disorder, includes but is not limited to any condition manifested as one or more physical and/or psychological symptoms for which treatment is desirable, and includes previously and newly identified diseases and other disorders.

The term “cancer” or “malignancy” are used interchangeably herein, refers to diseases that are characterized by uncontrolled, abnormal growth of cells which results in an increase in a particular cell type or increase in a tissue growth or tissue mass. Cancer cells can spread locally or through the bloodstream and lymphatic system to other parts of the body. The term is also intended to include any disease of an organ or tissue in mammals characterized by poorly controlled or uncontrolled multiplication of normal or abnormal cells in that tissue and its effect on the body as a whole. Cancer diseases within the scope of the definition comprise benign neoplasms, dysplasias, hyperplasias as well as neoplasms showing metastatic growth or any other transformations like e.g. leukoplakias which often precede a breakout of cancer.

As used herein, the term “tumor” refers to a mass of transformed cells that are characterized, at least in part, by containing angiogenic vasculature. The transformed cells are characterized by neoplastic uncontrolled cell multiplication which is rapid and continues even after the stimuli that initiated the new growth has ceased. The term “tumor” is used broadly to include the tumor parenchymal cells as well as the supporting stroma, including the angiogenic blood vessels that infiltrate the tumor parenchymal cell mass. Although a tumor generally is a malignant tumor, i.e., a cancer having the ability to metastasize (i.e. a metastatic tumor), a tumor also can be nonmalignant (i.e. non-metastatic tumor). Tumors are hallmarks of cancer, a neoplastic disease the natural course of which is fatal. Cancer cells exhibit the properties of invasion and metastasis and are highly anaplastic.

As used herein, the term “metastases” or “metastatic tumor” refers to a secondary tumor that grows separately elsewhere in the body from the primary tumor and has arisen from detached, transported cells, wherein the primary tumor is a solid tumor. The primary tumor, as used herein, refers to a tumor that originated in the location or organ in which it is present and did not metastasize to that location from another location. As used herein, a “malignant tumor” is one having the properties of invasion and metastasis and showing a high degree of anaplasia. Anaplasia is the reversion of cells to an immature or a less differentiated form, and it occurs in most malignant tumors.

The term “renal cell carcinoma” and “RCC” are used interchangeably herein, refers to a tumor of the kidney. Tumors of the kidney can be malignant or benign and are the most common primary malignant kidney tumor. RCC usually begins in the cells that line the small tubes of each nephron. Renal cell tumors can grow as a single mass, and can multiple RCC tumors can develop on a single kidney or both kidneys. The term RCC encompasses different subtypes of RCC, such as, but not limited to epithelial renal cell carcinoma (RCC), clear cell (conventional), papillary RCC (chromophil), chromophobe RCC, collecting duct RCC (<1%) and unclassified RCC subtypes.

The term “clear cell RCC” refers to the most common renal neoplasm seen in adults (70% of tumors derived from tubular epithelium). Clear cell RCC can be as small as 1 cm or less and discovered incidentally, or it can be as bulky as several kilograms, and often presents pain, as a palpable mass or with hematuria, but a wide variety of paraneoplastic syndromes have been described. Clear cell RCC might be clinically silent for years and may present with symptoms of metastasis. Clear cell RCC has a characteristic gross appearance; the tumor is solid, lobulated, and yellow, with variegation due to necrosis and hemorrhage, with in some instances, the tumor circumscribed, or invade the perirenal fat or the renal vein.

The term “therapeutically effective amount” refers to an amount that is sufficient to effect a therapeutically or prophylactically significant reduction in a symptom associated with an angiogenesis-mediated condition when administered to a typical subject who has an angiogenesis-mediated condition. A therapeutically or prophylatically significant reduction in a symptom is, e.g. about 10%, about 20%, about 30%, about 40%, about 50%, about 60%, about 70%, about 80%, about 90%, about 100%, about 125%, about 150% or more as compared to a control or non-treated subject. In some embodiments where the angiogenesis-mediated condition is cancer, the term “therapeutically effective amount” refers to the amount that is safe and sufficient to prevent or delay the development and further spread of metastases in cancer patients. The amount can also cure or cause the cancer to go into remission, slow the course of cancer progression, slow or inhibit tumor growth, slow or inhibit tumor metastasis, slow or inhibit the establishment of secondary tumors at metastatic sites, or inhibit the formation of new tumor metastasis.

The term “treat” or “treatment” refer to both therapeutic treatment and prophylactic or preventative measures, wherein the object is to prevent or slow down the development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with cancer as well as those likely to develop secondary tumors due to metastasis.

The term “subject” “patient” and “individual” are used interchangeably herein, and refer to an animal, for example a human, to whom treatment, including prophylactic treatment, with a composition as described herein, is provided. The term “mammal” is intended to encompass a singular “mammal” and plural “mammals,” and includes, but is not limited: to humans, primates such as apes, monkeys, orangutans, and chimpanzees; canids such as dogs and wolves; felids such as cats, lions, and tigers; equids such as horses, donkeys, and zebras, food animals such as cows, pigs, and sheep; ungulates such as deer and giraffes; rodents such as mice, rats, hamsters and guinea pigs; and bears. Preferably, the mammal is a human subject. As used herein, a “subject” refers to a mammal, preferably a human.

As used herein, the term “treating” includes reducing or alleviating at least one adverse effect or symptom of a condition, disease or disorder associated with cancer. As used herein, the term treating is used to refer to the reduction of a symptom and/or a biochemical marker of cancer by at least 10%. As a non-limiting example, a treatment can be measured by a change in a cancer stem cell biomarker as disclosed herein, for example a change in the expression level of a cancer stem cell biomarker by at least 10% in the direction closer to the reference expression level for that cancer stem cell biomarker.

The term “effective amount” as used herein refers to the amount of therapeutic agent or pharmaceutical composition to reduce or stop at least one symptom or marker of the disease or disorder, for example a symptom or marker of cancer. For example, an effective amount using the methods as disclosed herein would be considered as the amount sufficient to reduce a symptom or marker of the disease or disorder or cancer by at least 10%. An effective amount as used herein would also include an amount sufficient to prevent or delay the development of a symptom of the disease, alter the course of a symptom disease (for example but not limited to, slowing the progression of a symptom of the disease), or reverse a symptom of the disease.

The terms “treat” and “treatment” refer to both therapeutic treatment and prophylactic or preventative measures, wherein the object is to prevent or slow down the development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total). “Treatment” can also mean prolonging survival as compared to expected survival if not receiving treatment. Those in need of treatment include those already diagnosed with cancer as well as those likely to develop secondary tumors due to metastasis.

The terms “polypeptide” and “protein” are used interchangeably to refer to a polymer of amino acid residues, and are not limited to a minimum length. Peptides, oligopeptides, dimers, multimers, and the like, are also composed of linearly arranged amino acids linked by peptide bonds, and whether produced biologically, recombinantly, or synthetically and whether composed of naturally occurring or non-naturally occurring amino acids, are included within this definition. Both full-length proteins and fragments thereof are encompassed by the definition. The terms also include co-translational (e.g., signal peptide cleavage) and post-translational modifications of the polypeptide, such as, for example, disulfide-bond formation, glycosylation, acetylation, phosphorylation, proteolytic cleavage (e.g., cleavage by furins or metalloproteases), and the like. Furthermore, for purposes of the present invention, a “polypeptide” refers to a protein that includes modifications, such as deletions, additions, and substitutions (generally conservative in nature as would be known to a person in the art), to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods. Polypeptides or proteins are composed of linearly arranged amino acids linked by peptide bonds, but in contrast to peptides, has a well-defined conformation. Proteins, as opposed to peptides, generally consist of chains of 50 or more amino acids. For the purposes of the present invention, the term “peptide” as used herein typically refers to a sequence of amino acids of made up of a single chain of D- or L-amino acids or a mixture of D- and L-amino acids joined by peptide bonds. Generally, peptides contain at least two amino acid residues and are less than about 50 amino acids in length.

The terms “homology”, “identity” and “similarity” refer to the degree of sequence similarity between two peptides or between two optimally aligned nucleic acid molecules. Homology and identity can each be determined by comparing a position in each sequence which can be aligned for purposes of comparison. For example, it is based upon using a standard homology software in the default position, such as BLAST, version 2.2.14. When an equivalent position in the compared sequences is occupied by the same base or amino acid, then the molecules are identical at that position; when the equivalent site occupied by similar amino acid residues (e.g., similar in steric and/or electronic nature such as, for example conservative amino acid substitutions), then the molecules can be referred to as homologous (similar) at that position. Expression as a percentage of homology/similarity or identity refers to a function of the number of similar or identical amino acids at positions shared by the compared sequences, respectfully. A sequence which is “unrelated” or “non-homologous”shares less than 40% identity, though preferably less than 25% identity with the sequences as disclosed herein.

As used herein, the term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T. C, G. U. or I) or residue occurs in both sequences to yield the number of matched positions, dividing the number of matched positions by the total number of positions in the comparison window (i.e., the window size), and multiplying the result by 100 to yield the percentage of sequence identity.

The terms “substantial identity” as used herein denotes a characteristic of a polynucleotide or amino acid sequence, wherein the polynucleotide or amino acid comprises a sequence that has at least 85% sequence identity, preferably at least 90% to 95% sequence identity, more usually at least 99% sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which can include deletions or additions which total 20 percent or less of the reference sequence over the comparison window. The reference sequence can be a subset of a larger sequence. The term “similarity”, when used to describe a polypeptide, is determined by comparing the amino acid sequence and the conserved amino acid substitutes of one polypeptide to the sequence of a second polypeptide.

As used herein, the terms “homologous” or “homologues” are used interchangeably, and when used to describe a polynucleotide or polypeptide, indicates that two polynucleotides or polypeptides, or designated sequences thereof, when optimally aligned and compared, for example using BLAST, version 2.2.14 with default parameters for an alignment (see herein) are identical, with appropriate nucleotide insertions or deletions or amino-acid insertions or deletions, in at least 70% of the nucleotides, usually from about 75% to 99%, and more preferably at least about 98 to 99% of the nucleotides. The term “homolog” or “homologous” as used herein also refers to homology with respect to structure and/or function. With respect to sequence homology, sequences are homologs if they are at least 50%, at least 60 at least 70%, at least 80%, at least 90%, at least 95% identical, at least 97% identical, or at least 99% identical. Determination of homologs of the genes or peptides of the present invention can be easily ascertained by the skilled artisan.

The term “substantially homologous” refers to sequences that are at least 90%, at least 95% identical, at least 96%, identical at least 97% identical, at least 98% identical or at least 99% identical. Homologous sequences can be the same functional gene in different species. Determination of homologs of the genes or peptides of the present invention can be easily ascertained by the skilled artisan.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith and Waterman (Adv. Appl. Math. 2:482 (1981), which is incorporated by reference herein), by the homology alignment algorithm of Needleman and Wunsch (J. Mol. Biol. 48:443-53 (1970), which is incorporated by reference herein), by the search for similarity method of Pearson and Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)).

One example of a useful algorithm is PILEUP. PILEUP creates a multiple sequence alignment from a group of related sequences using progressive, pairwise alignments to show the percent sequence identity. It also plots a tree or dendogram showing the clustering relationships used to create the alignment. PILEUP uses a simplification of the progressive alignment method of Feng and Doolittle (J. Mol. Evol. 25:351-60 (1987), which is incorporated by reference herein). The method used is similar to the method described by Higgins and Sharp (Comput. Appl. Biosci. 5:151-53 (1989), which is incorporated by reference herein). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described by Altschul et al. (J. Mol. Biol. 215:403-410 (1990), which is incorporated by reference herein). (See also Zhang et al., Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff and Henikoff, Proc. Natl. Acad. Sci. USA 89:10915-9 (1992), which is incorporated by reference herein) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin and Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference amino acid sequence if the smallest sum probability in a comparison of the test amino acid to the reference amino acid is less than about 0.1, more typically less than about 0.01, and most typically less than about 0.001.

By “specifically binds” or “specific binding” is meant a compound or antibody that recognizes and binds a desired polypeptide but that does not substantially recognize and bind other molecules in a sample, for example, a biological sample, which naturally includes a polypeptide of the invention.

As used herein, the terms “administering,” and “introducing” are used interchangeably and refer to the placement of the agents as disclosed herein into a subject by a method or route which results in at least partial localization of the agents at a desired site. Compounds can be administered by any appropriate route which results in an effective treatment in the subject.

As used herein, the term “biological sample” refers to a cell or population of cells or a quantity of tissue or fluid from a subject. Most often, the sample has been removed from a subject, but the term “biological sample” can also refer to cells or tissue analyzed in vivo, i.e. without removal from the subject. Often, a “biological sample” will contain cells from the animal, but the term can also refer to non-cellular biological material, such as non-cellular fractions of blood, saliva, or urine, that can be used to measure gene expression levels. Biological samples include, but are not limited to, tissue biopsies, scrapes (e.g. buccal scrapes), whole blood, plasma, serum, urine, saliva, cell culture, or cerebrospinal fluid. Biological samples also include tissue biopsies, cell culture. A biological sample or tissue sample can refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the sample is from a resection, bronchoscopic biopsy, or core needle biopsy of a primary or metastatic tumor, or a cellblock from pleural fluid. In addition, fine needle aspirate samples are used. Samples may be either paraffin-embedded or frozen tissue. The sample can be obtained by removing a sample of cells from a subject, but can also be accomplished by using previously isolated cells (e.g. isolated by another person), or by performing the methods of the invention in vivo. Biological sample also refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the biological samples can be prepared, for example biological samples may be fresh, fixed, frozen, or embedded in paraffin.

By a “decrease” or “inhibition” used in the context of the level of expression or activity of a gene refers to a reduction in protein or nucleic acid level. For example, such a decrease may be due to reduced RNA stability, transcription, or translation, increased protein degradation, or RNA interference. Preferably, this decrease is at least about 5%, at least about 10%, at least about 25%, at least about 50%, at least about 75%, at least about 80%, or even at least about 90% of the level of expression or activity under control conditions.

By an “increase” or “higher” in the expression or activity of a gene or protein is meant a positive change in protein or nucleic acid level. For example, such a increase may be due to increased RNA stability, transcription, or translation, or decreased protein degradation. Preferably, this increase is at least 5%, at least about 10%, at least about 25%, at least about 50%, at least about 75%, at least about 80%, at least about 100%, at least about 200% (i.e. 2-fold), or at least about 500% (i.e. 5-fold), or at least about 10,000% (i.e. 10-fold) or more over the level of expression or activity under control conditions.

Compositions or methods “comprising” one or more recited elements may include other elements not specifically recited. For example, a composition that comprises a fibril component peptide encompasses both the isolated peptide and the peptide as a component of a larger polypeptide sequence. By way of further example, a composition that comprises elements A and B also encompasses a composition consisting of A, B and C.

The terms “comprising” means “including principally, but not necessary solely”. Furthermore, variation of the word “comprising”, such as “comprise” and “comprises”, have correspondingly varied meanings. The term “consisting essentially” means “including principally, but not necessary solely at least one”, and as such, is intended to mean a “selection of one or more, and in any combination.”

In this specification and the appended claims, the singular forms “a,” “an,” and “the” include plural references unless the context clearly dictates otherwise. Thus, for example, reference to a composition for delivering “a drug” includes reference to two or more drugs. In describing and claiming the present invention, the following terminology will be used in accordance with the definitions set out below.

This invention is further illustrated by the following example which should not be construed as limiting. The contents of all references cited throughout this application, as well as the figures and tables are incorporated herein by reference.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages can mean±1%.

### EXAMPLES

The examples presented herein relate to methods for diagnostic and prognostic methods of renal cell carcinoma (RCC) by analysis of gene group expression patterns in subjects. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Methods

Cell lines: The human RCC cell lines 786-0, UMRC2, UMRC3, UMRC6 (obtained through ATCC) lack wild-type VHL. Parental cell lines were used to derive isogenic clones 1) stably expressing HA-VHL30 or harboring empty pRC/CMV plasmid as a control and 2) stably inactivating HIF1a or HIF2a through shRNAi construct and their corresponding empty vector controls. Transfection were done with Lipofectamine 2000 using manufacturers instructions and cloned were generated by neomycin selection. Cells were grown in DMEM (Media Tech) with 10% Fetal Clone (Hyclone Laboratories) plus 1× penicillin-streptomycin-glutamine solution (100×, Invitrogen Life Sciences), supplemented with neomycin at the appropriate concentration for each cell line. Cells were grown to 80-90% confluency. These clones were labeled WT8 and PRC3 respectively. In addition, 780-0 clones stably expression an RNAi vector targeting HIF2a or empty vector control were also established (PTR and PTV respectively). To purify FABP6 we engineered U2OS cells stably expressing FABP6 tagged in the C-terminus with FLAG peptide or containing empty vector control. Cells were grown in DMEM (Media Tech) with 10% Fetal Clone (Hyclone Laboratories) supplemented with penicillin-streptomycin-glutamine solution (100×, Invitrogen Life Sciences). To condition tissue culture supernatant we applied 5 ml of phenol red free DMEM overnight onto the cells. Supernatant was collected, centrifuged at 2,000 RPM for 5 minutes and the aqueous phase transferred into a new vial and snap frozen.

Human Sample Collection and preparation: Patient samples were collected after informed consent of an IRB approved protocol was obtained. Part of each sample was frozen in liquid nitrogen immediately after surgery and stored at −80 C. The samples were made anonymous before the study.

Plasma, serum and urine from patients with renal cell carcinoma have been collected, under prior IRB approved protocol, before nephrectomy for localized disease and at regular intervals following nephrectomy. Blood samples are then spun at 3,500 rpm, aliquoted and stored at −80° C. until processing where they are thawed on ice. Tumor samples are also frozen and stored after pathology evaluation to determine histological subtyping. All patients have provided informed consent for tumor and blood sample collection.

RNA Extraction and QRT-PCR: Normal human RNA control samples were obtained from Stratagene. The tissues included kidney, liver, brain, skin, and spleen. RNA was isolated from cells at 80-90% confluence. Total RNA was isolated using TRIzol reagent (Life Technologies, Rockville, Md.) according to the manufacturer's instructions for cell lysis which was then transferred to 15 mL Falcon tubes and 1.2 mL of chloroform added. The mixture was centrifuged at 3000 rpm at 4° C. The aqueous fraction was collected and added to an equal volume of Diethyl Pyrocarbonate (DEPC)-treated 75% ETOH. RNA was extracted using RNeasy Mini Kit columns (Qiagen) according to the manufacturers' instructions and eluted into 30 μl DEPC-treated distilled water. RNA (1 μg total) was reverse transcribed to cDNA using Super Script III (RT Poly) according to Invitrogen's protocol.

QRT-PCR. Quantitative real-time PCR was performed per the manufacturer's recommendations using the Syber Green Detection System (BioRad). Intron spanning primers for CA9 were designed (forward: 5′-GAGGATCTACCTGGAGAGGA-3′ (SEQ ID NO:28); reverse: 5′-CTGGAAGCCCAGGAGTTCCA-3′ (SEQ ID NO:29)). Quantitation of B-globulin (forward 5′-TTT CAT CCA TCC GAC ATT GA-3′ (SEQ ID NO:30); reverse: 5′-ATC TTC AAA CCT CCA TGA TG-3′ (SEQ ID NO:31)) was performed and used for normalization of gene expression data. Each sample was run in triplicate. RNA sample without RT polymerase was used as a negative control. All PCR products were sequenced to confirm the identity of the amplified gene.

Microarray Analysis: Transcriptional profiling was done on HG-U133A Affymetrix GeneChips containing 22,283 genes. cDNA was prepared according to the manufacturer's protocol. Total RNA (8 Ag) was used in the first-strand cDNA synthesis with T7-(dT)24 primer (GGCCAGTGAATTGTAATACGACTCACTATAGGGAGGCGG-(dT)24 (SEQ ID NO: 13) and SuperScript II (Life Technologies). The second-strand cDNA synthesis was processed according to the manufacturer's protocol. Virtual northern blots were created for each potential biomarker candidate with the assistance of GeneCards (www.genecards.org) to query SAGE and UNIGENE databases.

Expression profiling and analysis: The cDNA samples, from three independent collections, were submitted for oligonucleotide array based analysis (Affymetrix). FILTER I refers to selection of candidate genes from cell culture gene expression analysis: specifically the inventors identified as candidate genes the genes which were upregulated (meaning selecting the genes that were above the standard deviation of their mean values) in cells lacking VHL as compared to their isogenic VHL reconstituted controls. Genes with higher fold upregulation got a higher score. FILTER I as used herein refers to the dependency of the gene regulation by VHL signaling (Signal dependent approach).

FILTER II refers to additional criteria by which the large number of genes generated by FILTER I was narrowed down. To narrow the pool of candidate biomarkers the inventors selected for the ones that expressed in a relatively restricted way in adult normal tissues. Specifically the inventors selected for the genes that are expressed significantly in two or less adult normal tissues (i.e. central nervous system and kidney only). This inventors used this second selection step in order to identify and select for genes with a restricted adult tissue expression pattern to enable easier identification of genes which will have a larger tumor-dependent incremental blood level changes and measurements of gene product changes (protein) in biological fluid obtained from patients and therefore will more accurately reflect changes attributed to RCC volume and/or activity. As used herein, this second selection step is termed this approach (FILTER II) organ restricted selection.

Validation of gene expression and QR-RTPCR. Normal human RNA control samples were obtained from Stratagene (kidney, liver, brain, skin, and spleen) and they were reversely transcribed to produce cDNA, as described above, for Quantitative real-time PCR (QRTPCR). Quantitative real-time PCR was performed per the manufacturer's recommendations using the Syber Green Detection System (BioRad). Intron spanning primers for the candidate markers were designed (reported biomarkers listed): CA12 (Forward—CTTGGCATCTGTATTGTGGT (SEQ ID NO:14); Reverse—GGGTGGCCATGGTCCCAAGG) (SEQ ID NO:15), EGLN3 (Forward—ATCAGCTTCCTCCTGTCCCT (SEQ ID NO:16), Reverse—GGGCTGCACTTCGTGTGGGT) (SEQ ID NO:17), FABP6 (Forward—TAGCAGACCC CTCAGCACCA (SEQ ID NO:18), Reverse—AGCTTCCCGCCCTCCATCTG) (SEQ ID NO:19), HIG2 (Forward—ACTCCTGCACGACCTGCTCC (SEQ ID NO:20), Reverse—TTATCACATGCTTCTGGATG (SEQ ID NO:21)), PMP22 (Forward—TCACTGGAATCTTCCAAATT (SEQ ID NO:22), Reverse—GTTTGAGTTTGGGATTTTGG (SEQ ID NO:23)), PNMA2 (Forward—ATGGATGCGGAGCGGAGGGC (SEQ ID NO:24), Reverse—CTCCACAGCCTCCTGGTCTC (SEQ ID NO:25)), and TNFSF7 (Forward—ATGCCGGAGG AGGGTTCGGG (SEQ ID NO:26), Reverse CGTAGCTGCCCCTTGTCCAG (SEQ ID NO:27)). Quantitation of β-globulin (Forward—TTTCATCCATCCGACATTGA (SEQ ID NO:30); Reverse—ATCTTCAAACCTCCATGATG; (SEQ ID NO:31)) was performed and used for normalization of gene expression data. Each sample was run in triplicate. Samples lacking RT polymerase during the RT reaction served as negative controls. All products of qRT-PCR reaction were initially identified by gel migration and further verified by sequencing.

Immunostaining. Formalin fixed and paraffin embedded human tissue was sectioned and the section was incubated with primary antibody at 1;1,000 dilution in PBS+0.5% Triton+1% albumin. Secondary goat-anti rabbit or goat-antimouse antibody from the DakoCytomation Envision anti-mouse system (DakoCytomation, Ely, UK) was applied for 30 min followed by five washes in PBS and developed by 5 min incubation in the presence of 3,30-diaminobenzidine (DAB [symbol] ) substrate. Positive staining results in a brown precipitate. Sections were counter-stained in Gills II haematoxylin (Surgipath Ltd, Richmond, Ill., USA) and mounted under glass coverslips using aqueous mounting medium (Faramount, DakoCytomation, Ely, UK).

Immunoprecipitations. Cells were lysed and proteins were immunoprecipitated as described before. FABP6-FLAG was immunoprecipitated using anti-FLAG antibody (M2, Sigma). Immunoprecipitates were washed with Tris/EDTA without detergent and FABP6 was eluted with 2.5 mM hydrochloric acid and neutralized and the eluate was neutralized with 0.1M Tris pH 8.0. Purified polyclonal antibodies recognizing human FABP6 were purchased from R&D, Minneapolis, Minn.

Western Blot Analysis. Cells were washed twice with ice cold Phosphate Buffered Saline (PBS) and lysed in RIPA buffer (50 mM Tris pH 8.0, 150 mM NaCl, 1% NP-40, 0.5% deoxycholate (DOC), 0.1% sodium dodecyl sulfate (SDS), and 0.02% NaN3) supplemented with proteinase inhibitors (20 ug/mL Trypsin Inhibitor, 10 μg/mL Leupeptin, 200 uM NaOrthovanadate, 5 μg/mL Pepstatin A, 20 μg/mL Aprotinin, 100 mM NaF and 200 μg/mL PMSF). Protein concentration was estimated by the Bradford method. Proteins were resolved (BioRad) by SDS-polyacrylamide gel electrophoresis (SDS-PAGE) and transferred onto PVDF membrane (BioRad) for immunoblotting.

Antibodies for Western Blot were used at indicated dilutions: anti-HIF-2a polyclonal (Novus NB100-122, 1:1,000), anti-actin monoclonal (Abcam Ab8226, 1:1,000), anti-HA monoclonal (12CA5, Abcam, 1:1,000), anti-carbonic anhydrase 9 monoclonal (M75, gift from Novartis, 1:3,000). Secondary horseradish peroxidase-conjugated antibodies to mouse or rabbit IgG were purchased from Pierce and detected by Western Lighting Chemiluminescence Reagent Plus (Perkin Elmer, NEL105) according to the manufacturer's protocol.

ELISA. ELISA of human plasma or serum for carbonic anhydrase 9 was performed in triplicate using the commercially available MN/CAIX ELISA kit (Siemens Diagnostics). Solid phase sandwich ELISA was performed per manufacture's protocol which has an analytic range of 0 pg/mL to 1500 pg/mL and a sensitivity of 2.5 pg/mL per product literature.

Selective Reaction Monitoring (SRM) for FABP6. Eluates from FABP6 containing immunoprecipitates were reduced and alkylate by adding Dithiothreitol (DTT) and Iodoacetamide and digested with trypsin. Peptides were desalted using a C18 reversed phase (RP) cartridge, eluted with 30% acetonitrile and concentrated by speed vacuum down to ˜20 microliters. Peptide mixture was fractionated by on-line nano flow LC/ESI-MS with 75 μm reversed-phase capillary columns containing a C18-HPLC packing material and sequenced by MS/MS data dependent acquisition using the LTQ-linear ion trap. To ID the peptides the inventors used the SEQUEST algorithm incorporated into the Bioworks software, version 3.2, (Thermo Electron, San Jose, Calif.). Search parameters included carbamidomethylation of cysteines, ±1.4 Daltons and ±1.0 Dalton tolerance for precursor and product ion masses, respectively. In an effort to minimize false positive identifications we used conservative criteria, and followed HUPO guidelines. SEQUEST results were ranked using correlation score (Xcorr) values: Xcorr 1.9, 2.2, 3.75 for singly, doubly and triply charged peptide ions, respectively, and all with dCn≦0.1. Furthermore, we validated the peptide identifications using peptide/protein Prophet™ (Keller, Nesvizhskii, 2002), a bayesian statistical algorithm that convert SEQUEST scores into probabilities. Peptide Identifications with high probability (≧95% confidence) were matched to proteins by searching the mouse and human databases.

Quantification of protein abundance. The relative abundance of a protein in a proteomic sample correlates well with the MS/MS spectra (=spectral count) acquired for the corresponding peptides; hence, initially relative protein quantification will be carried out based on the total number of spectral counts.

### Example 1

Identification of genes differentially expressed between and by differential gene expression analysis. The inventors identified a group of RCC biomarkers using differential gene expression analysis, and novel dual-selection criteria of selecting genes upregulated in RCC cell lines. The inventors compared the gene expression profile of human renal cell carcinoma cell lines 786-0, UMRC6 and 769-P, which are VHL-deficient human renal cell carcinoma cell lines (VHL deficiency is a hallmark of clear cell RCC) with human renal cell carcinoma cell lines in which have been transfected with a vector expressing the human VHL gene. The inventors compared the gene expression profile of the VHL-deficient RCC cell lines to those which express VHL and identified changes in the levels of gene expression (i.e. the gene expression profile) of a series of gene transcripts that were upregulated or increased in VHL-deficient human RCC cell lines as compared to RCC cell lines which express VHL.

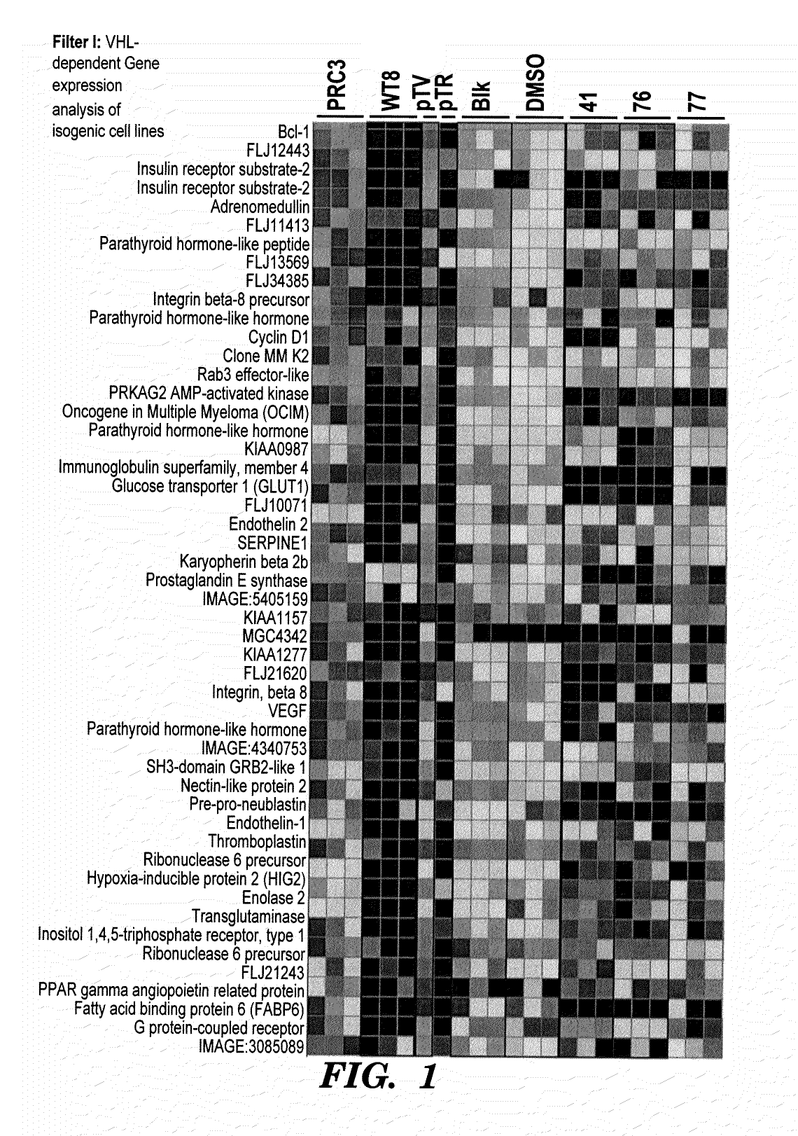

As disclosed herein, the first steps in identifying candidate RCC biomarkers required generating a master list of VHL regulated genes. To this end, the inventors compared the gene expression profile between the VHL deficient 786-O cells and their isogenic, VHL replete, counterparts (FIG. 1). The inventors initially selected the 100 genes that displayed the highest fold increase in the absence of VHL. Since loss-of-VHL is a hallmark of clear cell RCC the inventors selected polypeptides of these genes which are upregulated in RCC tumors.

The inventors then performed a second analysis by selecting genes that had a restricted normal adult tissue expression, to identify candidate biomarkers which are produced by RCC and may contribute significantly to their concentration in the blood. To apply this filter (Filter I) in the biomarker list, the inventors examined the relative tissue specificity of the VHL-dependent genes by querying the SAGE and UNIGENE databases, using the HUGO Gene nomenclature for the gene symbol. In addition, the inventors cross-referenced this information to the Novartis database. Thus, the inventors identified a subset of 12 candidate genes with relatively organ-restricted expression (significant expression in 2 or less organs other than kidney). Results from gene expression analysis, herein termed Filter I are shown in FIG. 1.

Accordingly to identify a group of genes that are specifically upregulated in RCC tissue samples the inventors applied two novel algorithms: the inventors selected genes that were (i) regulated by VHL and/or HIF and 2) have a restricted adult normal tissue expression pattern (i.e. restricted adult gene expression means that in normal adult tissue the gene is expressed only in a few organ types). This approach is different to the typical criteria used to select RCC biomarkers used previous studies, in which the selection criteria selects for genes encoding secreted proteins.

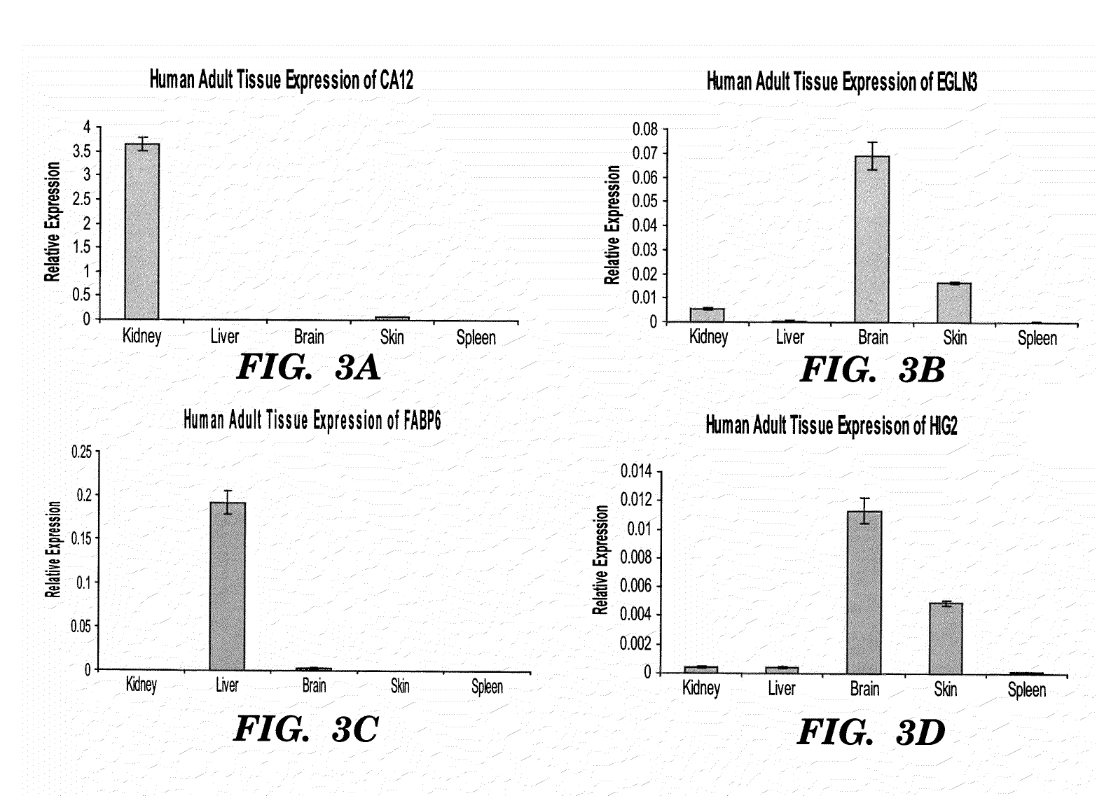

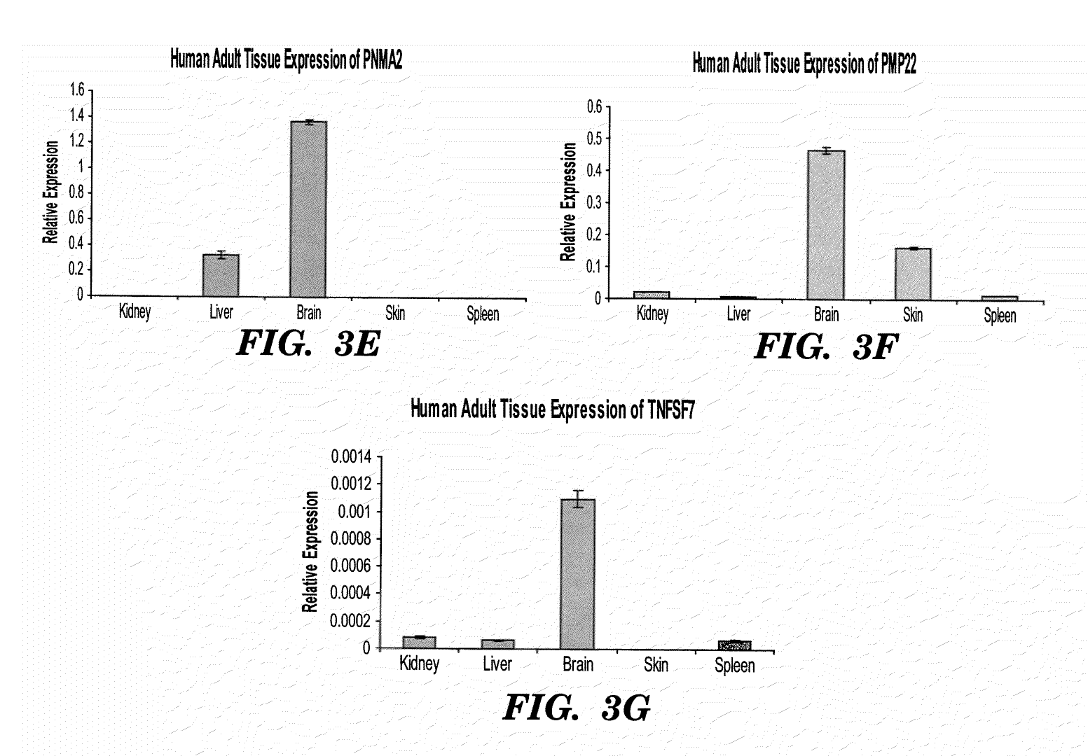

To further obtain experimental evidence for a restricted adult tissue expression of the candidate biomarkers the inventors examined their message in a panel of commercially available normal adult tissue total RNA collection (FIG. 3A-3G). This final criterion narrowed down the list to 6 candidate markers: Carbonic anyhdrase 12, EGLN homolog 3 (EGLN3), fatty acid binding protein 6 (FABP6), hypoxia inducible gene 2 (HIG2), peripheral myelin protein 22 (PMP22), paraneoplastic antigen 2 (PNMA2) and tumor necrosis factor (ligand) superfamily 7 (TNFSF7).

The inventors selected RCC biomarkers based on restricted tissue expression profile, as outlined as Filter II in the method section. For example, as shown in FIG. 3A-3G adult tissue expression of each of the 6 biomarkers examined and is limited only to a few tissue types of several tissue types examined, such as kidney, liver, brain and spleen. For example, EGLN3 is expressed significantly in adult brain and to a lesser extent in the skin (FIG. 3B). The relative expression of this marker in other tested tissues, including the liver (a usual site of pleiotropic gene expression) is very low. Therefore, the EGLN3 gene therefore fulfills the criteria for proposed biomarker; it is regulated by VHL-HIF axis (Filter I) and its adult normal expression is restricted to 2 or less tissues (Filter II).

By using the dual-selection criteria of selecting genes upregulated in RCC cell lines as compared to RCC cell lines comprising VHL, and selecting genes with restricted adult expression, the inventors identified a group of RCC biomarkers with clear target-to-noise ratio, as shown in Table 1.

### Example 2

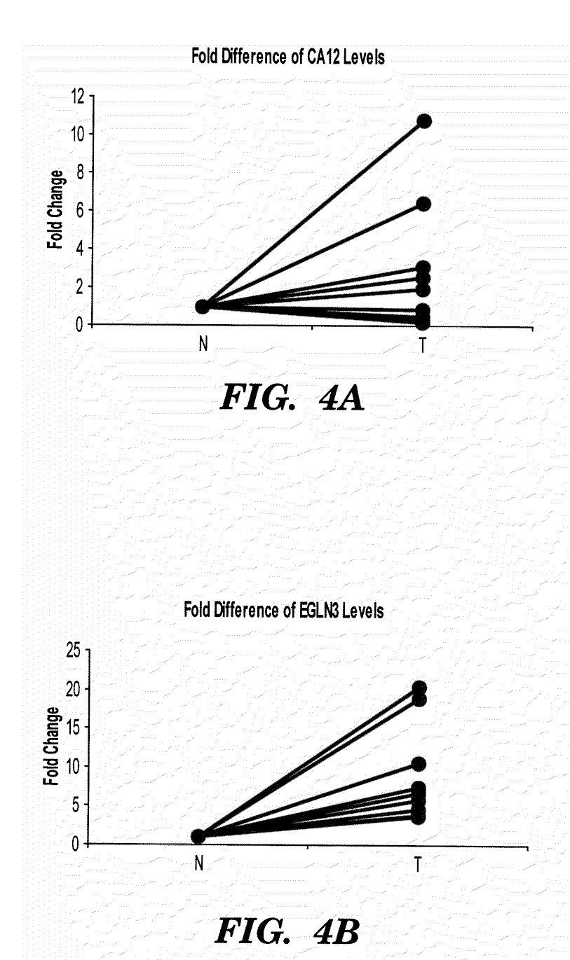

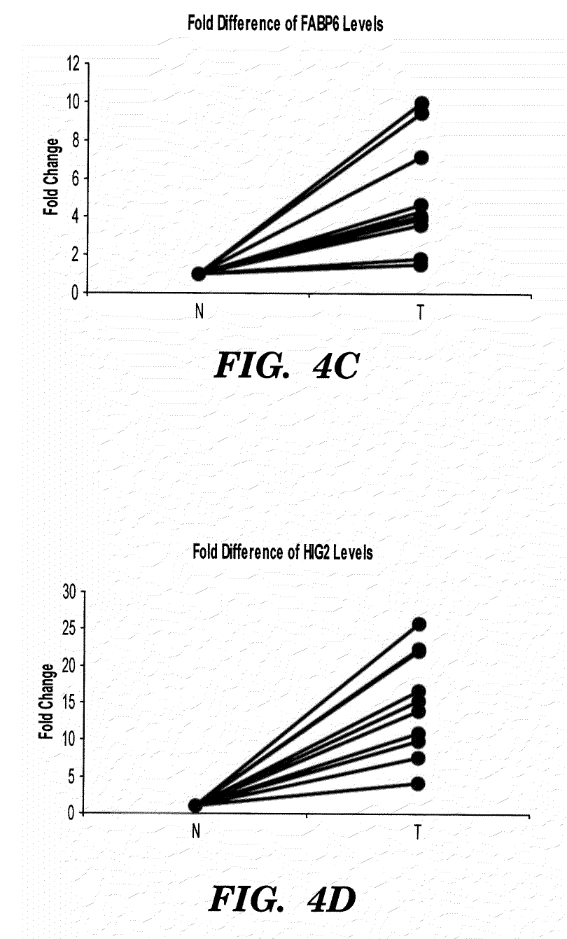

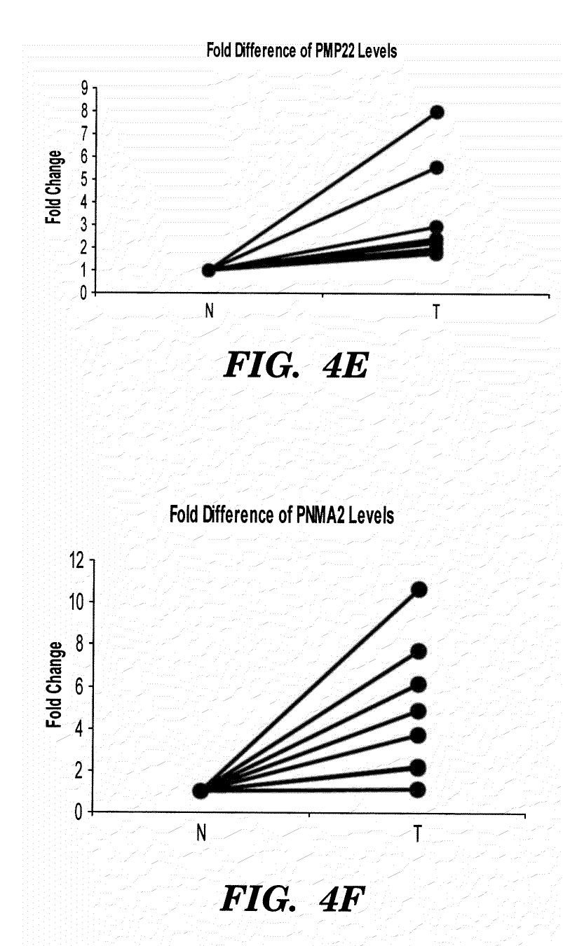

Validation of the group of RCC biomarkers. The inventors next assessed whether the expression of these selected genes (which had restricted adult tissue expression) was VHL-dependent in vitro and examined whether it was conferred through the VHL-regulated hypoxia inducible factor (HIF). For this purpose the inventors used the paired isogenic 786-O-derived cell lines in which VHL was replete or HIF2a inactivated through by specific shRNA (REF) and examined the expression of each candidate biomarker mRNA messages by qRT-PCR (FIGS. 4A-4F).

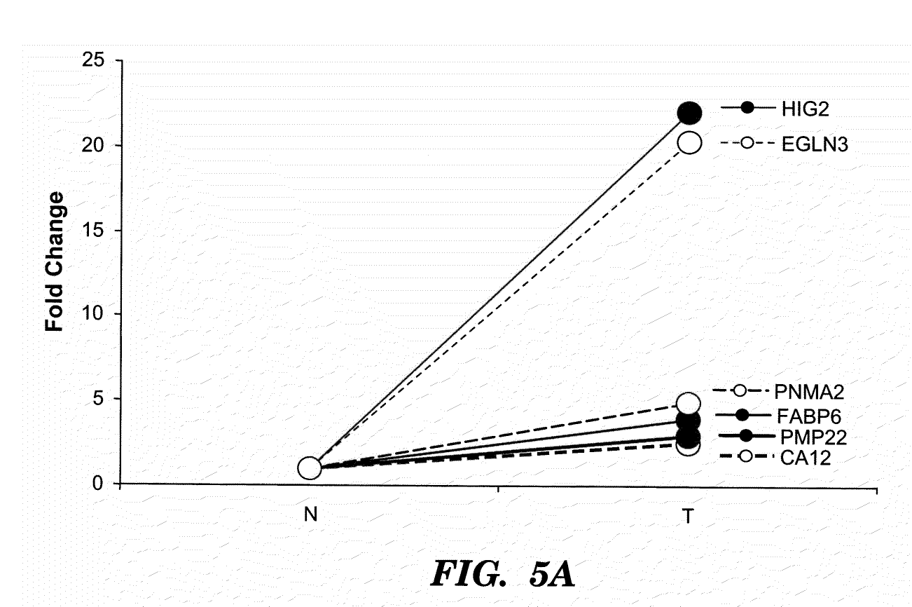

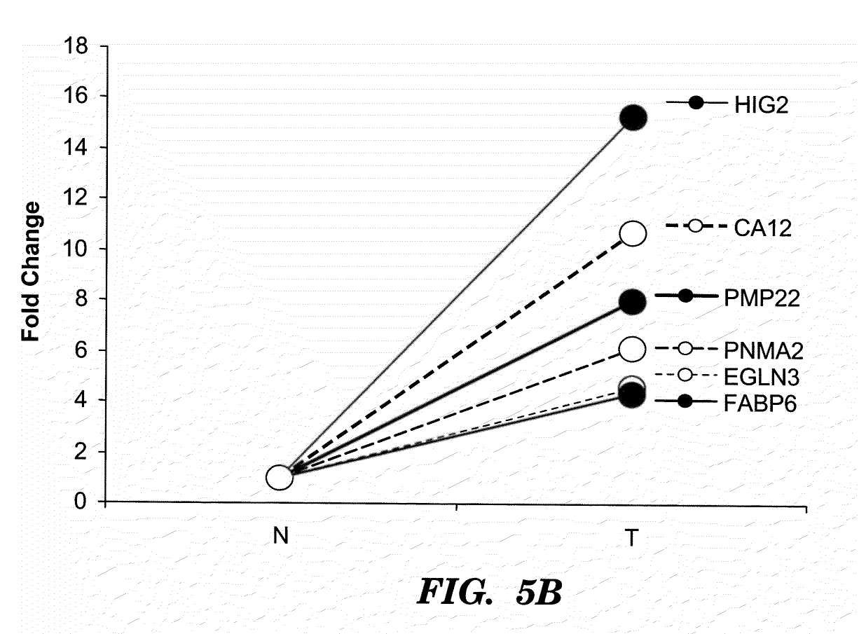

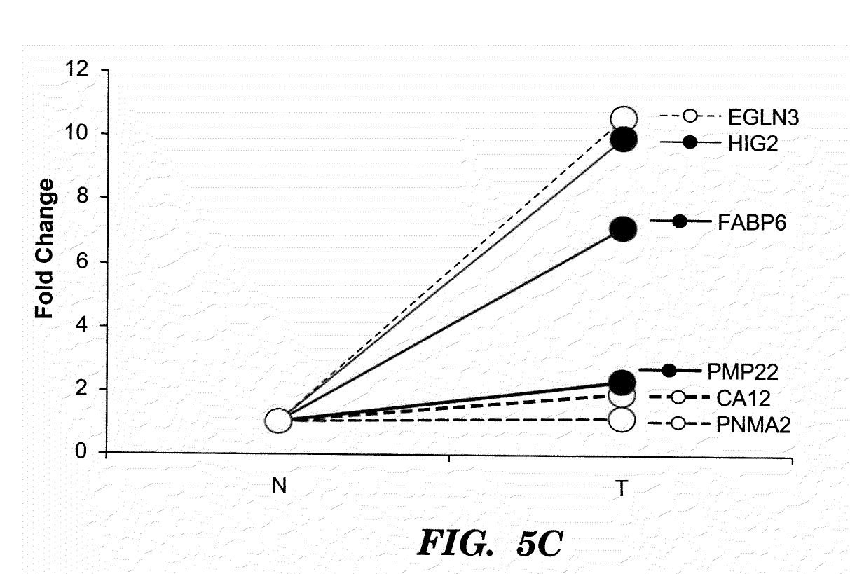

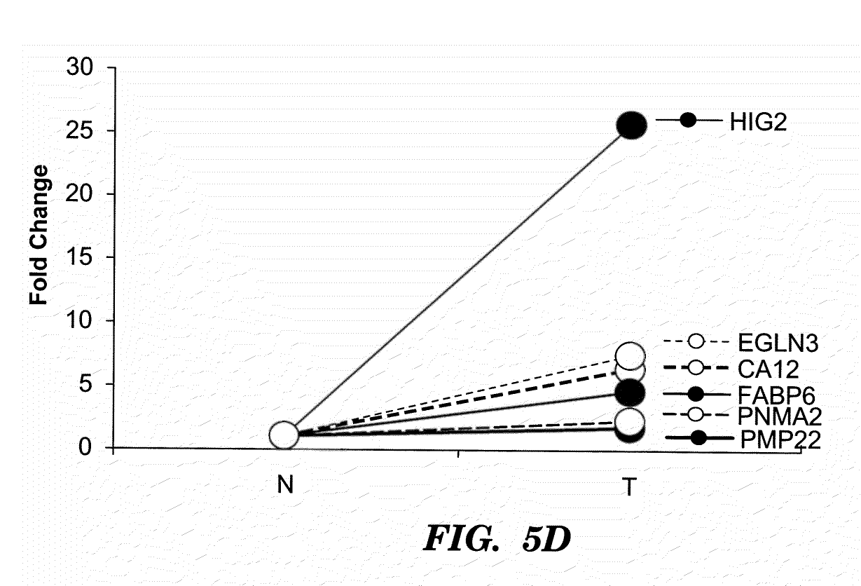

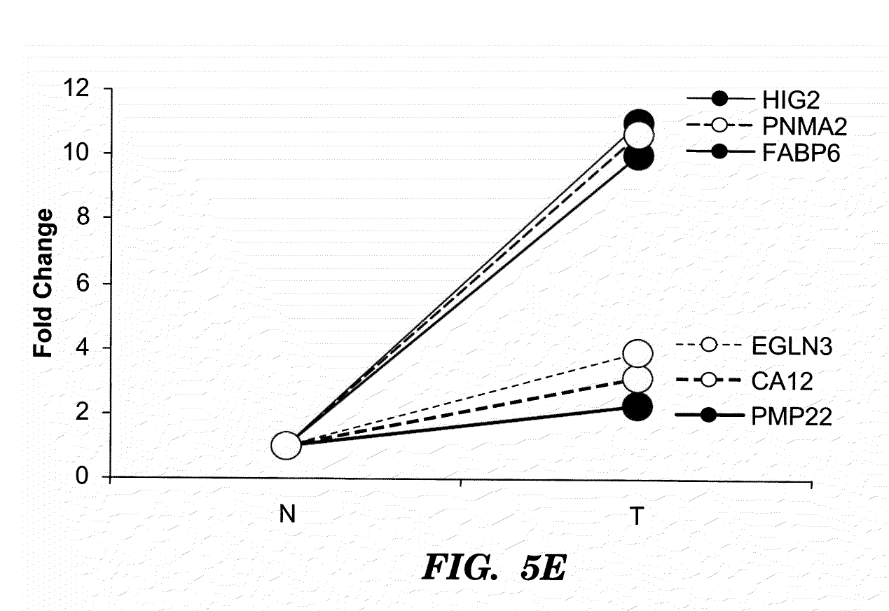

The inventors validated the RCC biomarkers in RCC tumor samples-paired expression analysis at the mRNA level between human RCC and normal renal parenchyma. The inventors validated the group of RCC biomarkers by assessing gene expression profile in normal kidney tissue and matched RCC tissue from the same subject (generated by Dr Towia Lieberman (BIDMC)). As shown in FIGS. 4 and 5, the inventors discovered that the mRNA expression of individual RCC biomarkers, such as PMP22 (FIG. 4E), PNMA2 (FIG. 4F), HIG (FIG. 4F), FABP6 (FIG. 4D), EGLN3 (FIG. 4C) and CA12 (FIG. 4A) is elevated in RCC samples as compared to matched normal kidney samples. The inventors discovered the RCC biomarkers are overexpressed in the majority of clear cell renal cell cancers tested.

The inventors further examined the 6 identified biomarkers in individual tumor samples and compared to normal matched tissue. Each tumor showed significant elevation in the majority of the candidate biomarkers but each tumor had a unique signature to the elevation pattern. HIG2 had the highest elevation in all but one tumor, which was notable for higher EGLN3 expression (FIG. 5). Expression of each biomarker using previously reported RCC Affymetrix GeneChip Data was examined in 10 clear cell RCC tumors and their matched normal tissue (FIG. 5). CA12 was significantly elevated in 6 out of 10 matched pairs with the highest fold increase greater than 10. PNMA2 was elevated in 9 out of 10 tumor/normal pairs with the highest demonstrating a 10-fold increase. TNFSF7 was elevated in 9 of the 10 pairs with 1.5-10 fold increase range. The remaining markers showed elevation in each matched pair although the magnitude of the change varied: EGLN3 (4-20× increase), FABP6 (1.5-10× increase), HIG2 (4-25× increase), and PMP22 (1.5-8× increase).

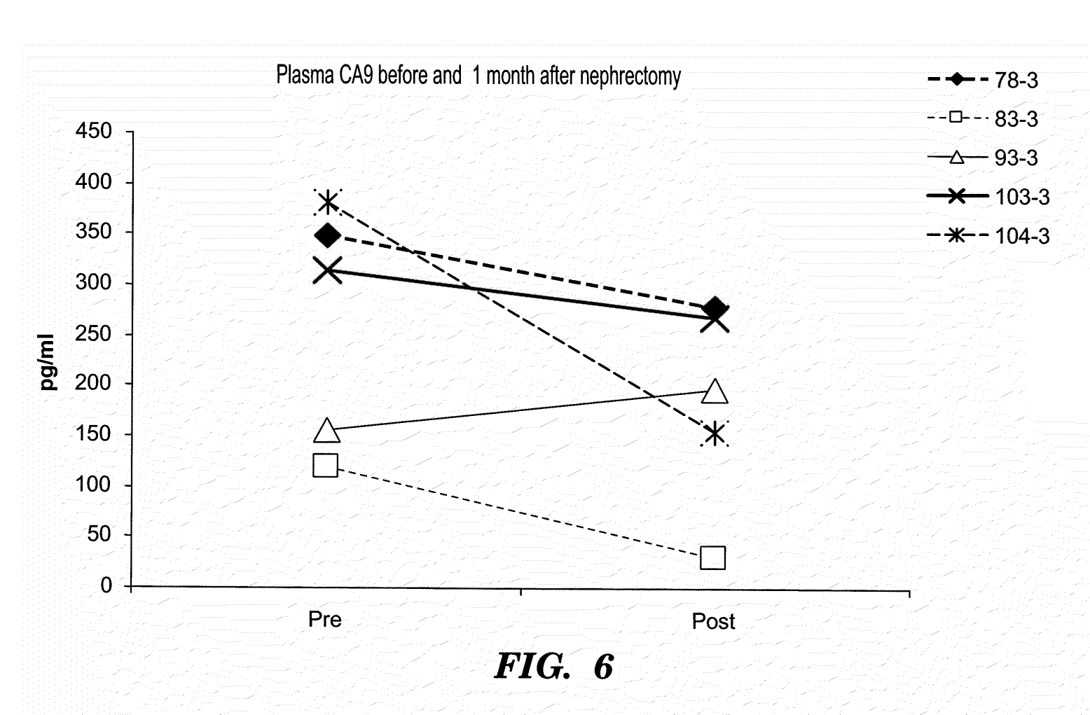

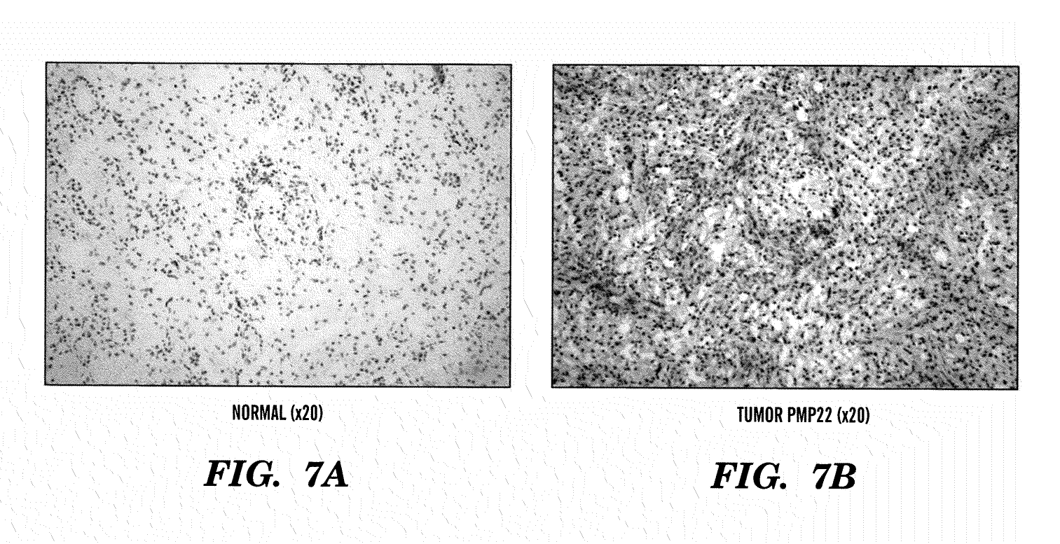

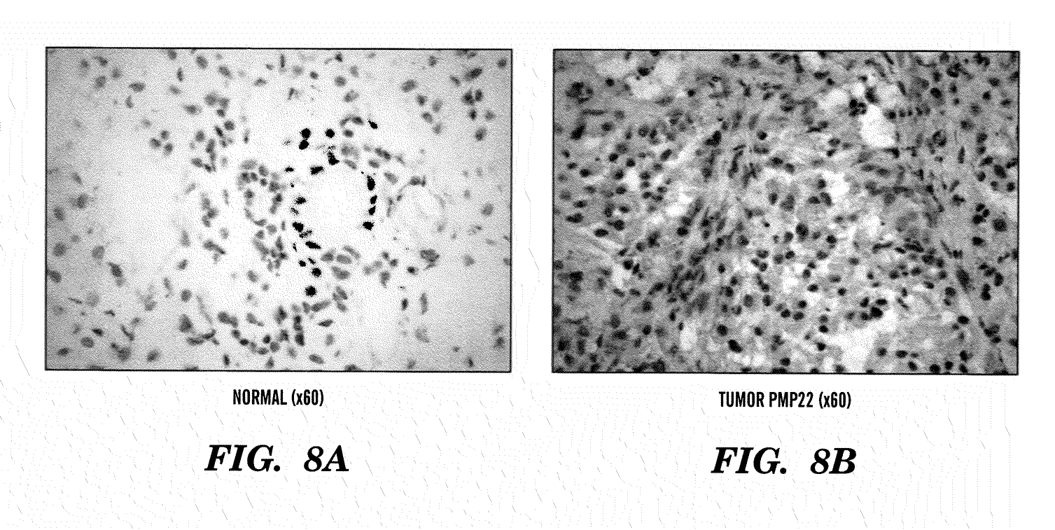

The inventors can also detect increased expression of RCC biomarkers in renal tissue from RCC tumor biopsy samples as compared to normal renal tissue, as shown by immunohistochemistry using an antibody against selective for the RCC biomarker PMP22 (FIGS. 7 and 8). The inventors also demonstrate that group RCC biomarkers are elevated in RCC (cRCC) samples as compared to matched normal (N) kidney samples, as shown in FIGS. 5 and 6.

The inventors then tested the protein levels of the one of the RCC biomarkers, CA9 in the plasma of patients with clear cell RCC before and following nephrectomy. As shown in FIG. 6, the presence of CA9 protein decreased in the plasma of a subjects following nephrectomy.

### Example 3

Validation of FABP6 as an exemplary RCC biomarkers. As demonstrated in Examples 1 and 2, the inventors have demonstrated that the panel of 6 biomarkers strongly indicates that the message of these 6 genes, initially identified in a signal dependent manner in cells, is upregulated in vitro (VHL deficient cell lines compared to VHL expressing) but also in vivo (human RCC tumors compared to matched controls).

The inventors then focused on fatty acid binding protein 6 (FABP6) as an exemplary biomarker for validation, and to further demonstrate that the identified biomarker polypeptides are useful biomarkers for RCC.

To evaluate FABP6 as a candidate biomarker the inventors accurately quantified its expression in RCC cell lines and in the plasma of patients with RCC. To be able to selectively tract and quantify FABP6 within complex biological fluids the inventors first obtained a peptide digest signature of the protein. Immunoprecipitated FABP6 was digested as described in the methods and the corresponding peptides identified by LC/ESI-MS. The inventors identified 7 corresponding peptides and selected 2 of them (based on favorable elution time and ionization properties) to track the protein (FIG. 5).

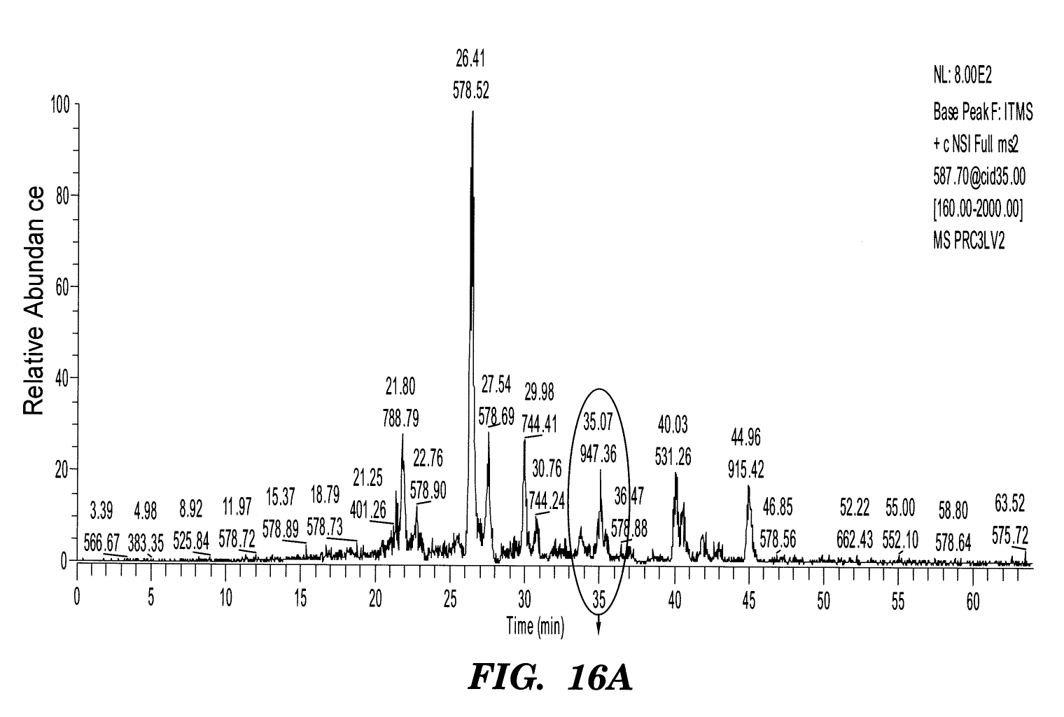

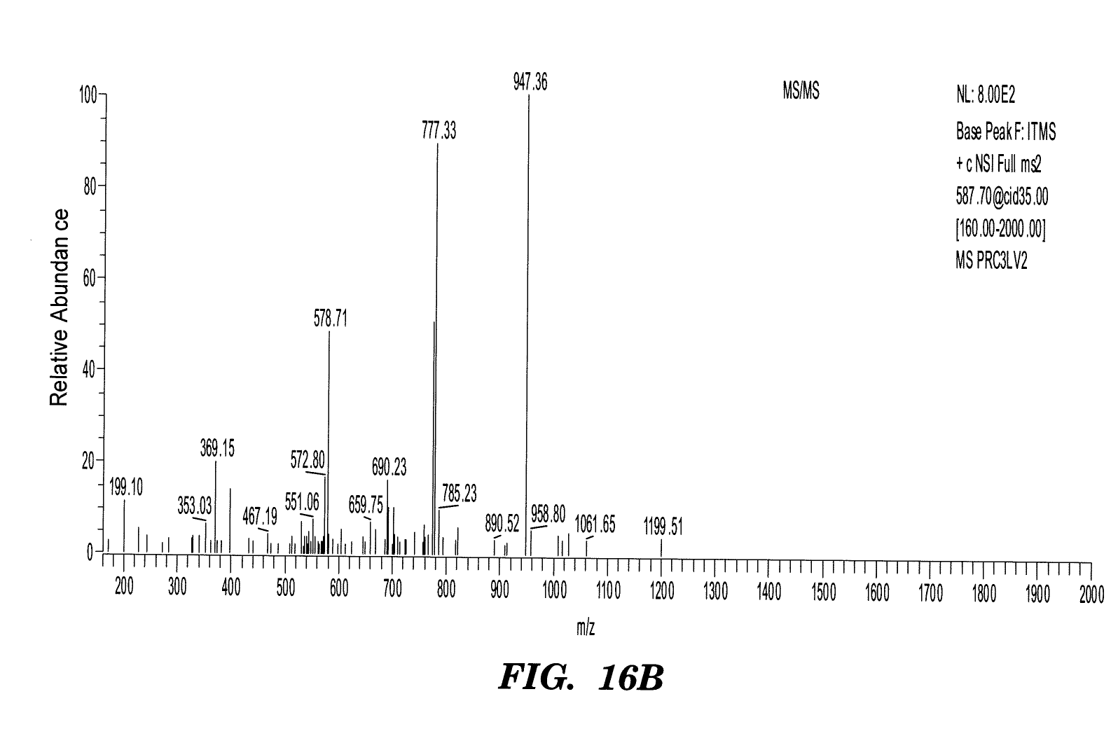

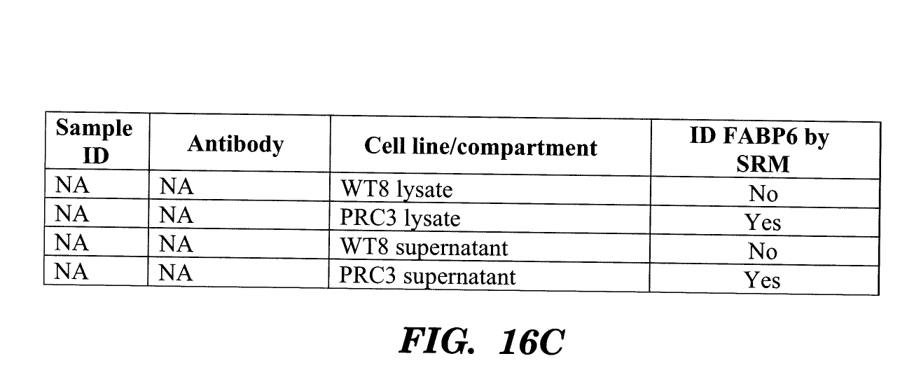

The inventors then applied this selected reaction monitoring (SRM) approach to the lysates of human RCC cell lines and to their corresponding conditioned tissue culture supernatant (FIG. 16). The inventors demonstrate that FABP6 is upregulated in the VHL deficient cells compared to their replete pair. In addition it is secreted in the tissue culture supernatant in a manner that reflects the intracellular differences. These observations confirm the expectation that message differences identified by the SIDOR approach are useful to identify differential expression of the biomarker at the protein level.

Lastly, to test whether in vitro regulation of FABP6 protein may support its value as a candidate biomarker, the inventors studied blood levels of FABP6 in RCC patients with localized disease prior to and after nephrectomy for localized disease. Application of FABP6 specific SRM in the plasma of these patients indicates that FABP6 blood levels are elevated in the presence of RCC (data not shown) and therefore strongly suggests that changes in FABP6 blood levels may reflect RCC activity. Sensitive detection of FABP6 by an immunometric assay may therefore serve as a peripheral blood test of RCC activity in specific populations at risk for developing the disease.

The inventors have demonstrated herein a translational approach to biomarker discovery. The inventors first profiled global gene changes induced by a disease-linked signal transduction pathway and we then selected the genes that have a relatively confined adult organ expression as potential biomarkers. The inventors validated the identified RCC biomarkers that are differentially regulated in RCC tumors. Finally, the inventors demonstrate by proteomic analysis for one of these candidate biomarkers, FABPF6, that the changes of FABPF6 protein in the blood of patients capture the activity of RCC tumor.

The inventors strategy is based on two postulates that constitute a novel approach to biomarker prioritization and identification: (1) Most of the proteins circulating in the plasma or secreted in the urine are not secreted proteins but are rather the product of cell death. It is of relevant interest that nuclear matrix proteins (NMPs) have been detected in the plasma of cancer patients and that the detection of one of them, NMP-22, in the urine is in clinical use for early diagnosis of bladder cancer (Black, 2006; Miller, 1992 #33) (2) proteins whose adult tissue expression is restricted mainly to the kidney may correspond to better changes in the plasma and or urine of ccRCC patients compared to proteins with pleiotropic tissue expression.

Loss-of-VHL tumor suppressor function and upregulation of its target transcription factor hypoxia inducible factor (HIF) is a hallmark of ccRCC. These are the earliest events detected during tumor development, even in premalignant lesions (Zhuang, 1995; Lubensky, 1996). Human VHL-deficient RCC cell lines recapitulate VHL-deficient renal cell carcinoma disease when injected in nude mice. The inventors have previously demonstrated that reintroduction of VHL into VHL-deficient cell lines or selective inactivation of HIF both lead to tumor suppression (Gnarra, 1996; Iliopoulos, 1995; Kondo, 2003; Kondo, 2002), and that VHL-dependent changes in these cell lines mimic the function of the VHL gene in vivo (Zimmer, 2004).

The inventors have identified 6 biomarkers for clear cell renal cell carcinoma via this SIDOR approach: Carbonic anyhdrase 12, EGLN homolog 3 (EGLN3), fatty acid binding protein 6 (FABP6), hypoxia inducible gene 2 (HIG2), peripheral myelin protein 22 (PMP22), paraneoplastic antigen 2 (PNMA2) and tumor necrosis factor (ligand) superfamily 7 (TNFSF7).

Upregulation of CA9 and HIG2 in RCC tumor tissue compared to normal kidney has been reported before but unlike the present invention, their value as diagnostic or prognostic biomarker when circulating in the plasma/and or urine of RCC patients was not known (Bui, 2004; Atkins, 2005; Liao, 1997).

Some of the RCC biomarkers identified by the inventors herein are linked to hypoxia-VHL-HIF signaling and/or cancer biology. Hypoxia inducible gene 2 (HIG2), a transcriptional target of HIF, is a secreted protein that activates cell growth and promotes transformation through stimulation of Wnt signaling (Kenny, 2005; Denko, 2000; Togashi, 2005). As demonstrated herein, HIG2 has been detected in the plasma of RCC patients. As demonstrated herein, HIG2 is one of a panel of RCC biomarkers which when assessed as a group or any combination of subgroup of RCC biomarkers as disclosed herein is useful for prognostic and diagnostic methods for subjects with RCC.

EGLN 3 is a human orthologue from the family of C. elegans EGL-9 oxygenases that prolylhydroxylate HIF (Bishop, 2004; Epstein, 2001). Human EGLN3 is also a HIF target through hypoxia response elements (HRE) located in the conserved region of the first intron (Taylor, 2001; Pescador, 2005; Aprelikova, 2004).

Fatty acid binding protein 6 (FABP6) is a direct target of HIF and was demonstrated to have one of the highest upregulation of expression in RCC cell lines. There is a leader peptide in the N-terminus of the protein and our data indicate that this protein is also actively secreted in the tissue culture supernatant. FABP6 is reported as a ileal protein with limited tissue expression (Fujita, 1995). FABP6 is over-expressed in early, primary colorectal tumors and adenomas but not in metastatic lymph nodes which may implicate an early role in carcinogenesis for FABP6 (Ohmachi, 2006). However, FABP6 has not been identified with RCC, nor has it been used as a biomarker for cancer in combination of any of the other RCC biomarkers as disclosed herein.

Peripheral myelin protein 22 (PMP22) is known to be involved in Charco-Marie-Tooth peripheral demyelinating diseases, and is expressed in adult peripheral and central nervous system (Warner, 1996; Roa, 1993). PMP22 is a HIF target localizing to the cytoplasm. Unlike the present application, comparative hybridization analysis (CGH) detected an increased copy number and amplification of PMP22 in osteosarcomas from 48 patients (Man, 2004), but PMP22 has not been identified to be upregulated in kidney cancer or RCC. The inventors have detected several isoforms of PMP22.

Paraneoplastic antigen 2 (PNMA2) is transmembrane protein involved in paraneoplastic limbic encephalopathy patients with breast and testicular cancer (Stich, 2007, Sutton, 2000). Unlike the present application, PNMA2 has not been identified with kidney cancer or RCC.

Tumor necrosis factor (ligand) superfamily 7 (TNFSF7) is a plasma circulating ligand for CD27 and exhibits a restricted adult tissue expression based on the inventors database search (Bowman, 1994; Hintzen, 1994; Goodwin, 1993). TNFSF7 has an important role in the renal cell carcinoma microenvironment, and has been previously reported to be up-regulated in RCC and hinders the lymphocyte response against tumor cells by inducing apoptosis in lymphocytes (Diegmann, 2006; Junker, 2005; Diegmann, 2005; Adam, 2006). However, TNFSF7 has not been used as a biomarker for RCC by determining its expression in biological samples from the subject (such as blood and urine), nor has it been used as a biomarker for RCC in combination of any of the other RCC biomarkers as disclosed herein.

The use of the SIDOR approach to identify a signal dependent (VHL signal pathway) and relative organ restricted panel of biomarkers may lead to biomarkers potentially sensitive and specific enough for clinical applications. The inventors have demonstrated and identified biomarkers for RCC.

### Example 4

CA9 as an exemplary example of the validation of a RCC Biomarker. Biomarkers for early detection of renal cell carcinoma (RCC) may help diagnose minimal residual disease in patients at risk for RCC, could guide anti-angiogenic therapy, may help identify patients for adjuvant therapy and could be used as surrogate markers of disease activity in Phase I/II trials. As disclosed herein, the inventors discovered that circulating blood levels of carbonic anhydrase 9 (CA9) correlates with RCC tumor burden and disease activity.

Efforts to discover and validate specific and sensitive biomarkers of disease activity in the blood and/or urine of RCC patients are underway. As disclosed herein, the inventors examined and validated the expression of carbonic anhydrase 9 (CA9) as a circulating blood biomarker of clear cell RCC activity. The majority of clear cell renal cell cancers are deficient in VHL function and they are characterized by upregulation of hypoxia inducible factors HIF1a and HIF2a. CA9 is a transmembrane glycoprotein involved in regulation of extracellular and intracellular pH. CA9 is a direct target of HIF1a, greatly upregulated in primary and metastatic RCC lesions [Liao, 1997, Wykoff, 2000]. CA9 has a restricted pattern of expression in normal tissues; it is detected in the epithelium of the stomach, gallbladder and exocrine pancreas. Retrospective analysis of patients treated with high dose IL2 indicated that CA9 may predict response to this therapy [Bui, 2004; Atkins, 2005; Li, 2007] and gene expression levels of CA9 in primary tumor may correlate with higher risk for metastatis [Li, 2007].

As CA9 is linked to hypoxia-HIF-VHL signaling and appears to have a restricted expression pattern in adult tissue, the inventors investigated its function as a blood circulating biomarker for RCC by measuring CA9 expression in the plasma/serum of patients with clinically localized disease pre- and post-nephrectomy. To the best of our knowledge, this is the first circulating biomarker reported to decrease in the blood of RCC patients after nephrectomy and/or to correlate with changes in tumor burden.

The inventors demonstrate that carbonic anhydrase 9 (CA) is a VHL-HIF target upregulated in clear cell RCC. The inventors used an anti-CA9 antibody (M75)-based ELISA test to measure CA9 levels in blood obtained before and after nephrectomy for clinically localized disease in: 1) Patients with clear cell RCC, 2) Patients with papillary and chromophobe RCC or oncocytoma, 3) Patients with benign kidney lesions and 4) Normal control individuals. The inventors discovered a significant decrease in the blood levels of CA9, after nephrectomy for localized disease, in the majority of patients with clear cell RCC (65% or 12/18). In contrast, patients with non-clear cell RCC, benign disease or undergoing debulking nephrectomy for metastatic disease had no decrease in CA9 blood levels after nephrectomy. Tumor volume correlated with pre-operative levels of CA9 (correlation coefficient r=0.77). Longitudinal follow up measurements of CA9 levels in a small group of patients indicated that rising CA9 levels correlate with disease progression. Plasma levels of CA9 in normal controls do overlap with pre-operative levels of CA9 in RCC patients prior to nephrectomy, indicating that it is unlikely that CA9 may be used as a single test for diagnosis of RCC in the general population.

Therefore, the inventors have discovered that plasma CA9 levels correlate with disease activity in clear cell RCC patients and can be used as a RCC biomarker development algorithms.

Loss-of-VHL function and constitutive upregulation of the transcription factor hypoxia inducible factor (HIF) is the earliest known molecular event in the majority of clear cell RCC. Reconstitution of the VHL function by stable reintroduction of VHL (either the 30 kDa or 19 kDa isoform) or inactivation of the HIF protein leads to growth suppression of these cell lines as tumors in the xenograft assay [Iliopoulos, 1995; Zimmer, 2004; Kondo, 2003]. These observations indicate that the fundamental signaling pathways implicated in renal carcinogenesis are intact in these cell lines.

In order to identify candidate biomarkers for RCC, the inventors compared the gene expression profile of human renal cell carcinoma cell lines that are deficient in VHL tumor suppressor protein to their isogenic counterparts that stably express VHL.

As disclosed in Example 1 and Example 2, one of the genes upregulated by loss-of-VHL function, as measured by DNA microarray analysis, was the transmembrane glycoprotein carbonic anhydrase 9 (CA9). To validate the microarray data, and to test whether differences at the mRNA level reflect differences in protein expression in various cell lines, the inventors examined CA9 mRNA and protein expression in human RCC cell lines under identical culture conditions.

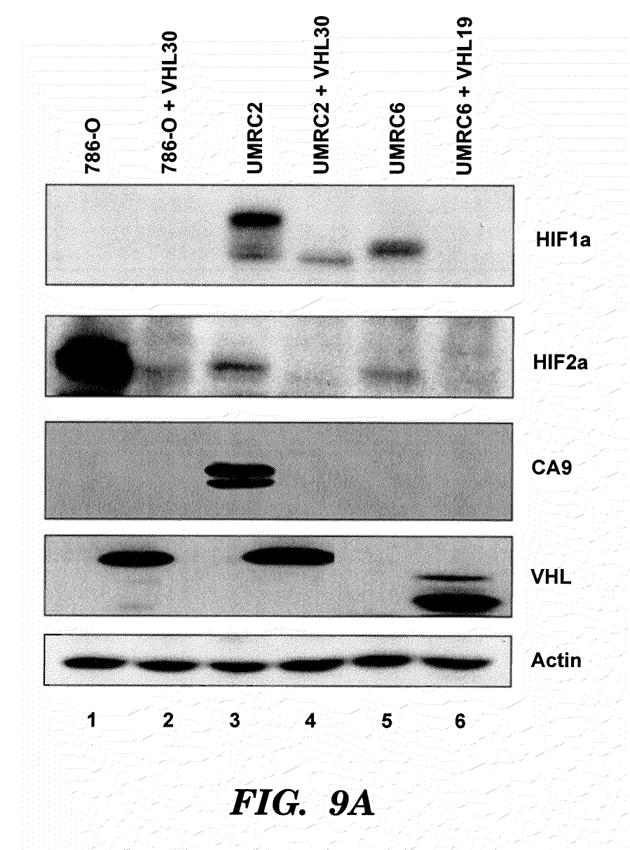

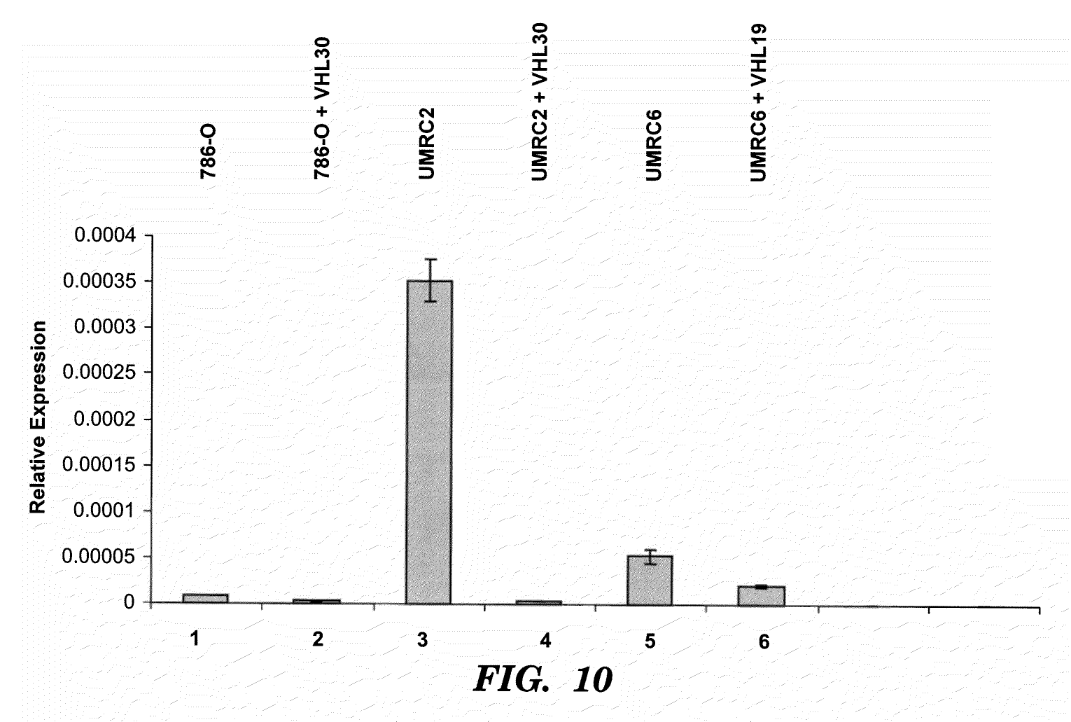

CA9 protein expression was analyzed by western blot (FIG. 9A) in VHL deficient cell lines 786-O, UMRC2 and UMRC6 (lanes 1, 3 and 5 of FIG. 9A) and their corresponding isogenic clones that express VHL30 or VHL19 (FIG. 9A, lanes 2, 4 and 6). CA9 mRNA expression was examined by qRT-PCR (FIG. 10). The high expression of the CA9 message in UMRC2 cells (FIG. 10, lane 3) corresponds to a robust signal for CA9 in western blot analysis (FIG. 9A, lane 3), while the much weaker mRNA expression in lines 786-O and UMRC6 (FIG. 10, lanes 1 and 5) resulted in no detectable protein (FIG. 9A, lanes 1 and 5). These experiments confirmed the inventors discovery can be used as a CA9 is a biomarker for RCC.

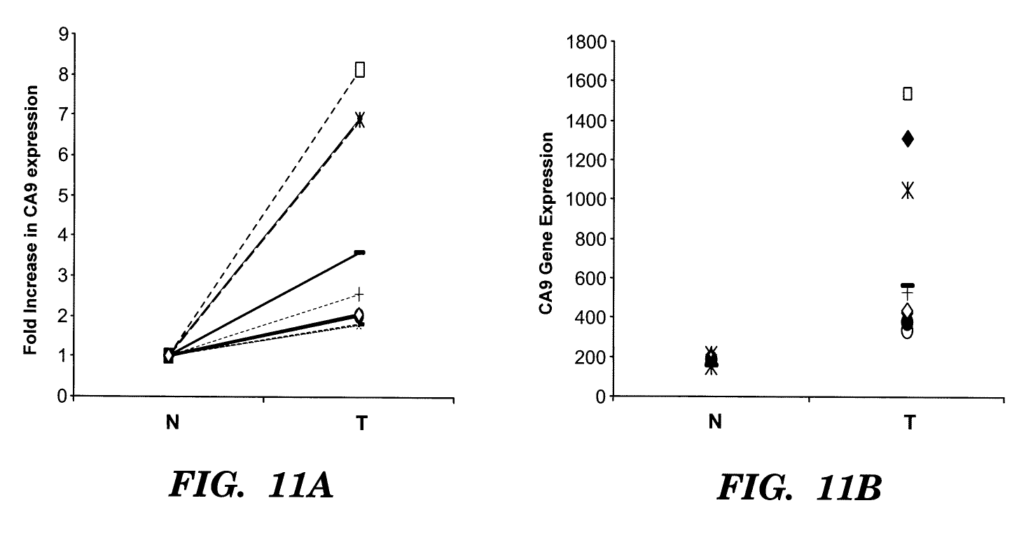

Next, the inventors assessed the expression of CA9 in matched tumor-normal tissue samples as well as adult normal tissues, by comparing the expression of CA9 by oligonucleotide microarray in 10 specimens of clear cell RCC tumor (T) to matched normal renal parenchyma (N) obtained from the same individuals. CA9 was discovered to be upregulated in all RCC specimens compared to normal matched tissue (FIG. 11A). Moreover, there was essentially no overlap between absolute values of mRNA signal detected in RCC tumors (T) compared to normal matched parenchyma (N) (FIG. 11B).

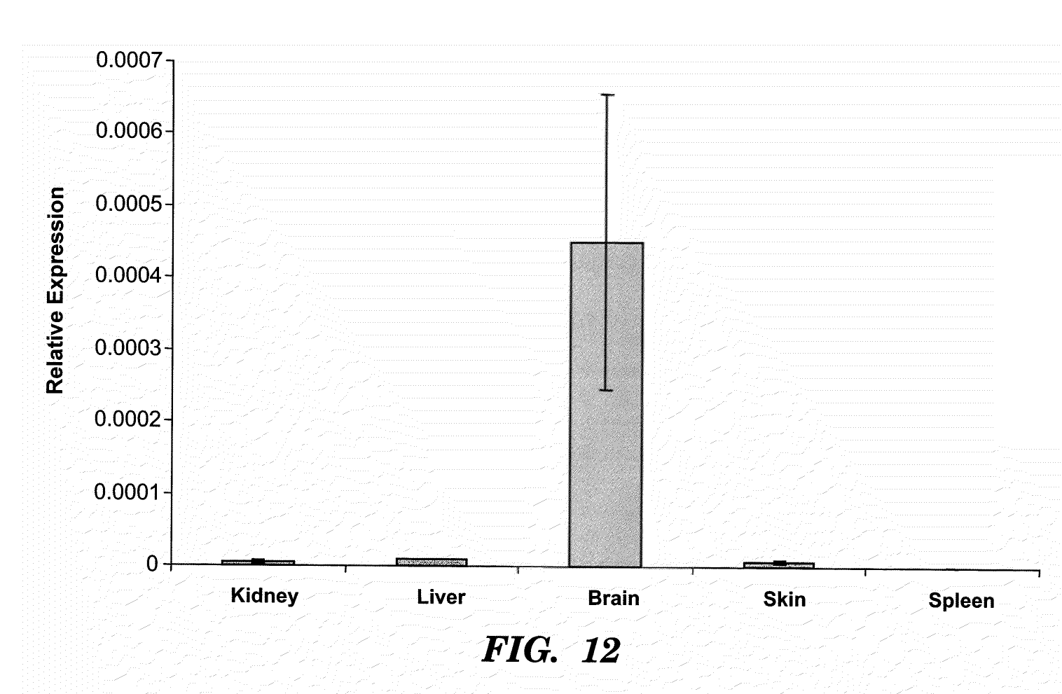

Changes in circulating levels of a biomarker attributed to the presence of RCC may be potentially masked because of its pleiotropic expression in other tissues. Organ-restricted expression of a candidate biomarker may allow for easier detection of incremental changes that correlate with tumor volume. The inventors therefore examined the expression of CA9 by qRT-PCR in total RNA extracted from various adult tissues (FIG. 12). CA9 was highly expressed in human adult brain compared to the other organs examined. Skin and liver, which are bulky organs, showed weak expression of CA9. In keeping with previous literature reports and our own microarray data, CA9 expression was very low in normal adult kidney.

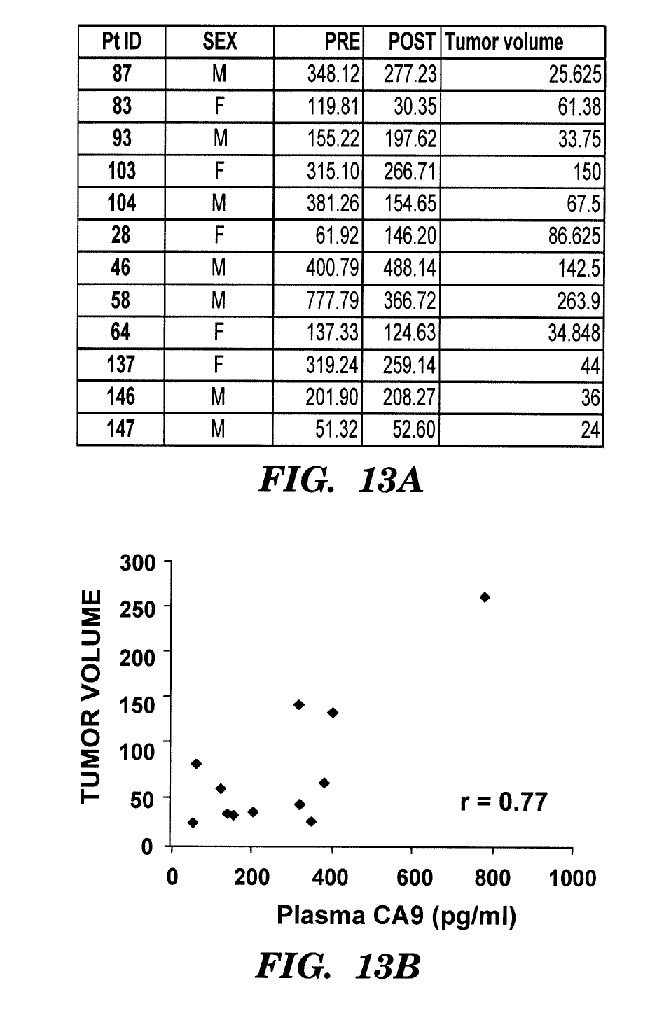

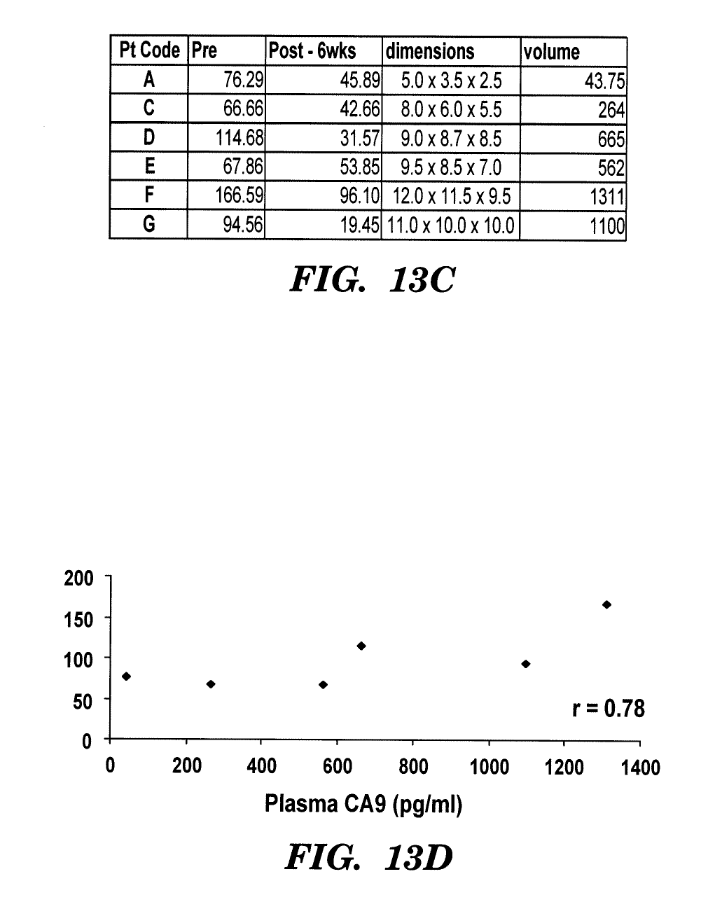

To examine weather circulating CA9 levels correlated with RCC disease activity, the inventors first measured the levels of this potential biomarker in the plasma of RCC patients with localized disease. Samples were obtained both prior to nephrectomy and between 6-8 weeks post surgery. FIG. 13A presents patient sex and tumor volume at a single institution (Massachusetts General Hospital) and circulating CA9 levels prior to (pre) and after (post) nephrectomy. Measurements were obtained with a commercially available CA9 ELISA kit (Siemens Diagnostics Inc.). The manufacturer reports, and the inventors independently confirmed, an 10% intra-assay variability. Thus, changes greater than 10% in either direction were regarded as significant. All measurements were done in triplicate and the reported values are the mean. All tumors reported in FIG. 11 are clear cell carcinomas. Six out of twelve patients (50%) had a decrease in post-operative values of CA9 (50%). In three out of twelve patients (25%), there was an increase, and the remaining 3 patients (25%) had no significant difference. Pre-operative values correlated with tumor volume (FIG. 12B, r=0.77).

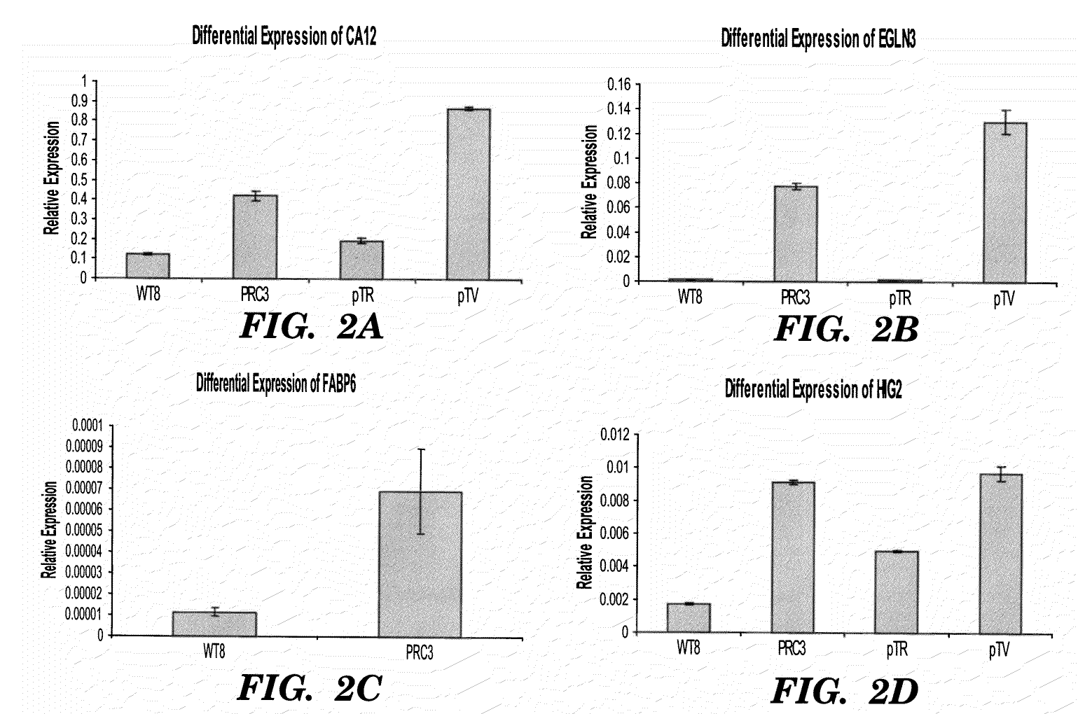

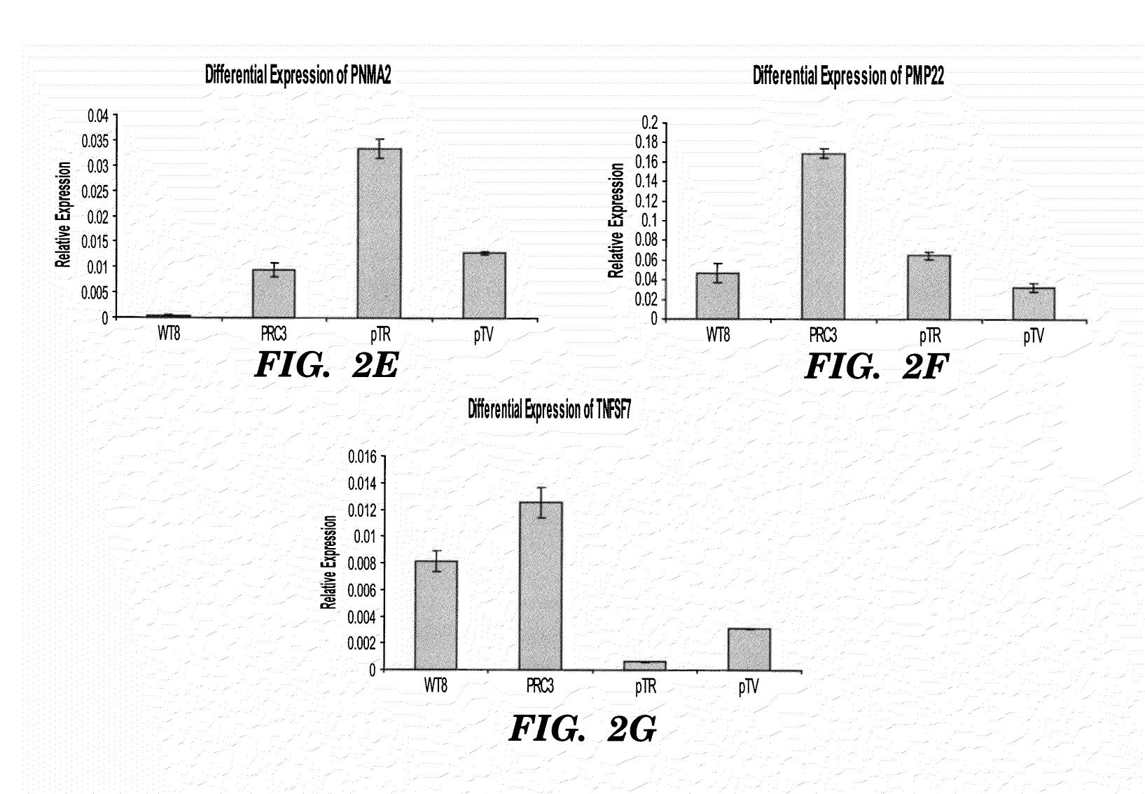

To test whether these changes reflected any institutional bias, the inventors obtained samples of patients independently collected using a similar protocol (the exception being that serum was banked instead of plasma) at a second institution (MD Anderson Cancer Center-MDACC). According to manufacturer of the assay (and also tested by the inventors independently) there is no difference between measurements of CA9 in plasma and serum. FIG. 12C describes the MDACC patient sample and the CA9 values prior to and after nephrectomy. Pre-operative values correlated with tumor volume (FIG. 2D, r=0.78). CA9 values decreased in all 6 patients post nephrectomy (100%). Analyzed together the data from both Institutions indicate that in a subset of patients with localized clear cell RCC (65.5%) undergoing curative nephrectomy, there is a decrease in the circulating levels of CA9 post-operatively.

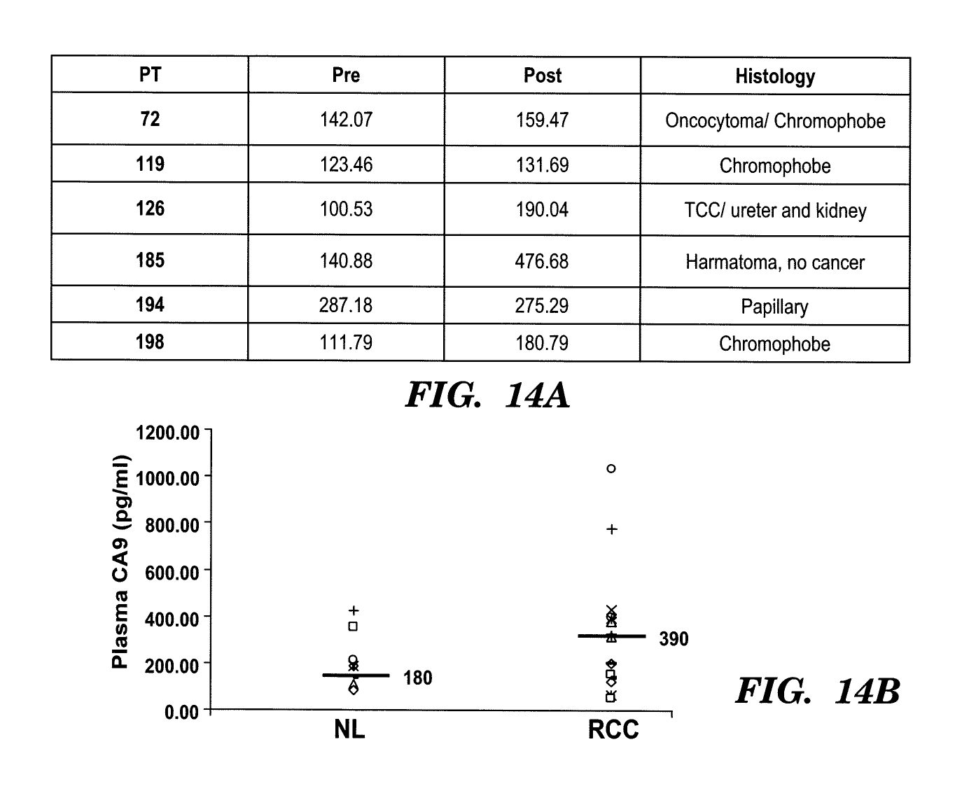

In contrast to patients with clear cell histology, nephrectomy for benign tumor or those of non-clear cell RCC histology did not result in a significant decrease in plasma CA9 levels (FIG. 14A). The inventors also measure CA9 in the plasma of patients without known RCC, as a sample of “normal control” individuals; there is significant overlap in the CA9 plasma values between normal controls and RCC patients prior to nephrectomy (FIG. 14B).

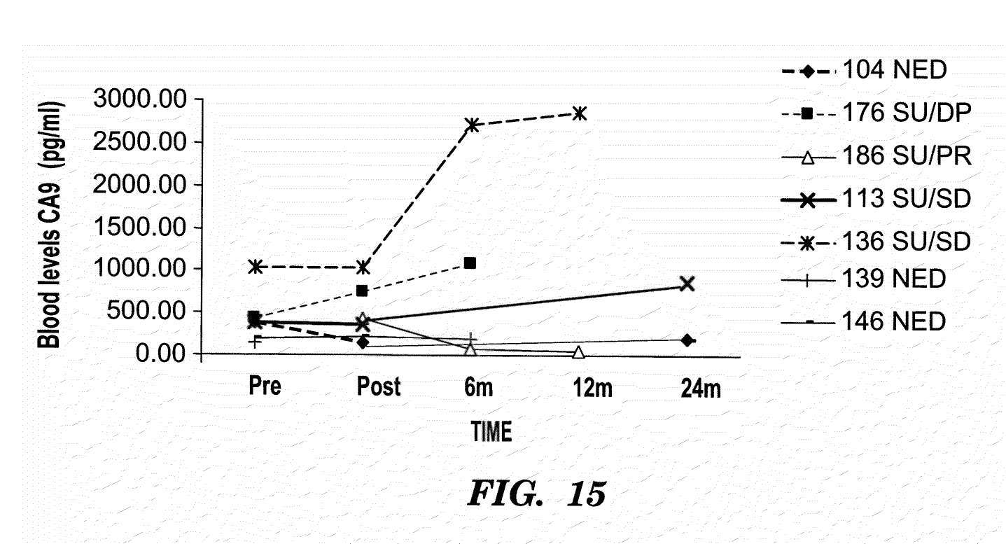

To examine whether blood levels of CA9 correlate with disease activity over time and/or herald a local or systemic disease relapse, the inventors measured and determined circulating CA9 levels in available plasma samples of clear cell RCC patients following curative or debulking nephrectomy (FIG. 15 and Table 4). Patients 104, 139 and 146 remained disease free at the indicated time of follow up and their longitudinal CA9 plasma levels did not rise above post or pre-operative levels. Patients 176, 186, 113 and 136 presented with metastatic disease and underwent debulking nephrectomy. It is notable that in the three patients that can be evaluated, the post-operative levels of CA9 did not decline. Patient 186 responded to treatment and the clinical response correlated with a marked decline in CA9 plasma levels. The remaining patients had either stable disease under treatment with anti-angiogenic agents or disease progression. In the latter case, plasma levels of CA9 rose steadily.

TABLE 4
Longitudinal measurements of plasma levels of CA9 in patients with clear cell
RCC undergoing curative or debulking nephrectomy.

| | | | | | 12 | | | FOLLOW |
|---|---|---|---|---|---|---|---|---|
| Pt | SEX | Pre | Post | 6 months | months | 2 years | STAGE | UP |
| 104 | M | 381.26 | 154.65 | | | 201.2812444 | pT1b Nx | NED |
| | | | | | | | Mx | May 10, 2007 |
| | | Apr. 21, 2005 | Jun. 23, 2005 | | | May 10, 2007 | | |
| 176 | M | 431.69 | 751.74 | 1087.08 | | | T1bN2M1 | Tx with |
| | | | | | | | | SU/DP |
| | | Pre | Post | Post | | | | |
| | | Dec. 13, 2006 | Feb. 15, 2007 | May 10, 2007 | | | | |
| 186 | M | NO | 430.28 | 70.55 | 52.43 | | T3bN2M0, | Tx with |
| | | PRE | | | | | DN | SU + G/PR |
| | | | | | | | Jan. 2007 | |
| | | | Post | Post | Post | | | |
| | | | Feb. 27, 2007 | Apr. 19, 2007 | May 10, 2007 | | | |
| 113 | M | 394.42 | 359.81 | | | 860.55 | T2N2M1 | Txwith |
| | | | | | | | on | SU/SD |
| | | | | | | | presentation | |
| | | Jul. 8, 2005 | Aug. 22, 2005 | | | Mar. 29, 2007 | | |
| 136 | F | 1032.19 | 1035.50 | 2711.44 | 2855.51 | | T3aN0M1 | SU |
| | | | | | | | | (June 2006)/SD |
| | | Mar. 30, 2006 | May 11, 2006 | Oct. 24, 2006 | Feb. 27, 2007 | | | |
| 139 | F | 143.28 | | 201.90 | | | T3aN0M0 | NED |
| | | | | | | | | (December 2006) |
| | | May 1, 2006 | | Dec. 19, 2006 | | | | |
| 146 | M | 201.90 | 208.27 | 203.17 | | | T1bN0M0 | NED |
| | | Jun. 8, 2006 | Aug. 24, 2006 | Jan. 16, 2007 | | | | |

SU = treatment with suten,
G = treatment with gemcitabine,
PR = partial response,
SD = stable disease,
DP = disease progression,
NED = no evidence of disease.
Specific values and times of blood collection are provided in Table 1.

As disclosed herein, the inventors have discovered that circulating blood levels of CA9, a transcriptional target of HIF activity, declined in 65% of clear cell RCC patients who underwent curative nephrectomy for organ confined disease. RCC patients with non-clear cell histology or benign tumors had no change in CA9 plasma levels. Pre-operative levels of CA9 correlated with tumor volume in patients with localized disease. None of the patients that presented with clinically overt metastatic disease and underwent debulking nephrectomy had a decrease in post-operative CA9 plasma levels. Moreover, follow up measurement of CA9 plasma levels correlated with tumor progression or response to therapy in the small group of patients the inventors examined.

CA9 levels were also discovered to be decreased post-operatively in a defined subset of patients with localized disease. Using the CA9 biomarker discovered by the inventors, patients with clear cell RCC can be divided in “high” and “low” expressers of CA9 by immunocytochemistry of the tumor.

The inventors also have discovered that CA12, another member of the CA family, is also overexpressed in clear cell RCC, albeit in a lower percentage of patients than CA9.

The inventors also discovered that all patients with progressing disease had increased CA9 plasma levels, above that observed post- and pre-operatively. One patient treated for systemic disease had a partial clinical response that correlated with reduced CA9 levels. Incidental timing of blood collection in one patient allowed the inventors to document an early rise in plasma levels of CA9 six months before detectable relapse, which demonstrated that in a given population of RCC patients, fluctuation in CA9 levels over time can be used as a biomarker for early detection of impeding disease relapse or progression.

Few other biomarkers for RCC have been proposed in small patient samples. Of those, kidney injury molecule-1 (KIM-1), a transmembrane glycoprotein upregulated in ischemic injury of the kidney epithelium, was reported to be elevated in RCC tumors and in the urine of RCC patients compared to normal controls [Han, 2002]. No measurements of KIM-1 in the blood of RCC patients before or following nephrectomy have been reported so far. Elevated levels of nuclear matrix protein 22 has been detected in the urine of patients with RCC and a follow up study indicated that specificity and sensitivity of RCC diagnosis ranged at 55% [Huang, 2000]. No data for blood levels of NMP22 have been reported. Matrix metalloproteinase activity has also been reported to be elevated in the urine of RCC patients [Sherief, 2003]. Other reported biomarkers of RCC in the blood include a tumor-specific isoform of Pyruvate Kinase (a HIF target) as well as plasma levels of IL-6, TNFa and circulating mRNA levels of prostate specific membrane antigen [Wechsel, 1999; Yoshida, 2002; Mulders, 2003; de la Taille, 2000]. Few of these biomarkers have limited specificity and may reflect a systemic response to tumor burden. Others such as KIM-1, TuPK and NMP22 could be incorporated in large clinical studies evaluating the performance of a group of disease specific biomarkers [Oremek, 2000].

In summary, the inventors have discovered, using gene expression comparison of signaling dependent human renal cell carcinoma cell lines, circulating biomarkers for clear cell RCC. As an exemplary example, the inventors tested one identified biomarker for RCC, CA9 and demonstrated that this biomarker correlates with the presence of the disease in a subset of clear cell RCC patients. To the best of our knowledge, this is the first report of blood changes in a potential RCC biomarker post nephrectomy for localize RCC disease.

### REFERENCES

The references cited herein and throughout the application are incorporated herein by reference.
Jemal, A., et al., Cancer statistics, 2007. CA Cancer J Clin, 2007. 57 (1): p. 43-66.
2. Motzer, R. J., N. H. Bander, and D. M. Nanus, Renal Cell Carcinoma. NEJM, 1996. 335 (12): p. 865-875.
3. Jones, J., et al., Gene signatures of progression and metastasis in renal cell cancer. Clin Cancer Res, 2005. 11 (16): p. 5730-9.
4. Black, P. C., G. A. Brown, and C. P. Dinney, Molecular markers of urothelial cancer and their use in the monitoring of superficial urothelial cancer. J Clin Oncol, 2006. 24 (35): p. 5528-35.
5. Miller, T. E., et al., Detection of nuclear matrix proteins in serum from cancer patients. Cancer Res, 1992. 52 (2): p. 422-7.
6. Zhuang, Z., et al., A microdissection technique for archival DNA analysis of specific cell populations in lesions <1 mm in size. Am J Pathol, 1995. 146 (3): p. 620-5.
7. Lubensky, I. A., et al., Allelic deletions of the VHL gene detected in multiple microscopic clear cell renal lesions in von Hippel-Lindau disease patients. American Journal of Pathology, 1996. 149: p. 2089-2094.
8. Kibel, A., et al., Binding of the von Hippel-Lindau tumor suppressor protein to elongin B and C. Science, 1995. 269: p. 1444-1446.
9. Jiang, Y., et al., Gene Expression Profiling in a Renal Cell Carcinoma Cell Line: Dissecting VHL and Hypoxia-Dependent Pathways. Mol Cancer Res, 2003. 1 (6): p. 453-62.
10. Ivan, M., et al., Biochemical purification and pharmacological inhibition of a mammalian prolyl hydroxylase acting on hypoxia-inducible factor. Proc Natl Acad Sci, 2002. 99 (21): p. 13459-64.
11. Mizukami, Y., et al., Hypoxia-inducible factor-1-independent regulation of vascular endothelial growth factor by hypoxia in colon cancer. Cancer Res, 2004. 64 (5): p. 1765-72.
12. Bui, M. H., et al., Prognostic value of carbonic anhydrase IX and KI67 as predictors of survival for renal clear cell carcinoma. J Urol, 2004. 171 ((6 Pt 1)): p. 2461-6.
13. Atkins, M., et al., Carbonic anhydrase IX expression predicts outcome of interleukin 2 therapy for renal cancer. Clin Cancer Res, 2005. 11 (10): p. 3714-21.
14. Liao, S. Y., et al., Identification of the MN/CA9 protein as a reliable diagnostic biomarker of clear cell carcinoma of the kidney. Cancer Res, 1997. 57 (14): p. 2827-31.
15. Kenny, P. A., T. Enver, and A. Ashworth, Receptor and secreted targets of Wnt-1/beta-catenin signalling in mouse mammary epithelial cells. BMC Cancer, 2005. 5: p. 3.
16. Denko, N., et al., Epigenetic regulation of gene expression in cervical cancer cells by the tumor microenvironment. Clin Cancer Res, 2000. 6 (2): p. 480-7.
17. Togashi, A., et al., Hypoxia-inducible protein 2 (HIG2), a novel diagnostic marker for renal cell carcinoma and potential target for molecular therapy. Cancer Res, 2005. 65 (11): p. 4817-26.
18. Bishop, T., et al., Genetic analysis of pathways regulated by the von Hippel-Lindau tumor suppressor in Caenorhabditis elegans. PLoS Biol, 2004. 2 (10): p. e289.
19. Epstein, A. C., et al., C. elegans EGL-9 and mammalian homologs define a family of dioxygenases that regulate HIF by prolyl hydroxylation. Cell, 2001. 107 (1): p. 43-54.
20. Taylor, M. S., Characterization and comparative analysis of the EGLN gene family. Gene, 2001. 275 (1): p. 125-32.
21. Pescador, N., et al., Identification of a functional hypoxia-responsive element that regulates the expression of the egl nine homologue 3 (egln3/phd3) gene. Biochem J, 2005. 390 (Pt 1): p. 189-97.
22. Aprelikova, O., et al., Regulation of HIF prolyl hydroxylases by hypoxia-inducible factors. J Cell Biochem, 2004. 92 (3): p. 491-501.
23. Fujita, M., et al., Molecular cloning, expression, and characterization of a human intestinal 15-kDa protein. Eur J Biochem, 1995. 233 (2): p. 406-13.
24. Ohmachi, T., et al., Fatty acid binding protein 6 is overexpressed in colorectal cancer. Clin Cancer Res, 2006. 12 (17): p. 5090-5.
25. Warner, L. E., B. B. Roa, and J. R. Lupski, Absence of PMP22 coding region mutations in CMT1A duplication patients: further evidence supporting gene dosage as a mechanism for Charcot-Marie-Tooth disease type 1A. Hum Mutat, 1996. 8 (4): p. 362-5.
26. Roa, B. B., et al., Charcot-Marie-Tooth disease type 1A. Association with a spontaneous point mutation in the PMP22 gene. N Engl J Med, 1993. 329 (2): p. 96-101.
27. Stewart, S. L., et al., Cancer mortality surveillance: United States, 1990-2000. MMWR Surveill Summ, 2004. 53 (3): p. 1-108.
28. Stich, O., et al., Specific antibody index in cerebrospinal fluid from patients with central and peripheral paraneoplastic neurological syndromes. J Neuroimmunol, 2007. 183 (1-2): p. 220-4.
29. Sutton, I., et al., Limbic encephalitis and antibodies to Ma2: a paraneoplastic presentation of breast cancer. J Neurol Neurosurg Psychiatry, 2000. 69 (2): p. 266-8.
30. Bowman, M. R., et al., The cloning of CD70 and its identification as the ligand for CD27. J Immunol, 1994. 152 (4): p. 1756-61.
31. Hintzen, R. Q., et al., Characterization of the human CD27 ligand, a novel member of the TNF gene family. J Immunol, 1994. 152 (4): p. 1762-73.
32. Goodwin, R. G., et al., Molecular and biological characterization of a ligand for CD27 defines a new family of cytokines with homology to tumor necrosis factor. Cell, 1993. 73 (3): p. 447-56.
33. Diegmann, J., et al., Immune escape for renal cell carcinoma: CD70 mediates apoptosis in lymphocytes. Neoplasia, 2006. 8 (11): p. 933-8.
34. Junker, K., et al., CD70: a new tumor specific biomarker for renal cell carcinoma. J Urol, 2005. 173 (6): p. 2150-3.
35. Adam, P. J., et al., CD70 (TNFSF7) is expressed at high prevalence in renal cell carcinomas and is rapidly internalised on antibody binding. Br J Cancer, 2006. 95 (3): p. 298-306.

1. (canceled)

2. A method for identifying a subject having increased likelihood of developing or having renal cell carcinoma (RCC) the method comprising:
(a) measuring the level of gene transcript expression or protein expression of a gene group wherein the gene group comprises at least three genes selected from a group of genes encoding; CA12; CA9; EGLN3; HIG2; TGFB3; NMU; PMP22; PNMA2; TNFRSF7; FABP6; CD70 (CD27L) or NPY1 in a biological sample obtained from a subject;
(b) comparing the level of gene transcript expression or protein expression of the same genes as measured in the biological sample from the subject in step (a) to a reference level;
wherein a higher level of the gene transcript expression or protein expression of the selected gene in the biological sample from the subject as compared the gene transcript expression or protein expression of the reference level indicates the subject is at increased risk of having or developing RCC.

3. A method for monitoring the progression of renal cell carcinoma (RCC) in a subject having, or likely of developing renal cell carcinoma (RCC), the method comprising:
(a) measuring the level of gene transcript expression or protein expression of a gene group wherein the gene group comprises of at least three genes selected from a group of genes comprising; CA12; CA9; EGLN3; HIG2; TGFB3; NMU; PMP22; PNMA2; TNFRSF7; FABP6; NPY1; CD70 (CD27L) in a biological sample obtained from a subject at a first time point;
(b) measuring the level of gene transcript expression or protein expression of at least three of the same genes as measured in step (a) in a biological sample obtained from a subject at a second time point;
(c) comparing the level of gene transcript expression or protein expression of the same genes as measured in the biological sample from the first time point with the level of gene transcript expression or protein expression in the biological sample from the second timepoint;
wherein a change in the level of the gene transcript expression or protein expression of at least three genes in the selected gene group in the biological sample from the subject at the first time point as compared to the level of gene transcript expression or protein expression of at least three of the same genes in the biological sample from the subject at the second timepoint indicates an alteration in the rate of progression of RCC in the subject.

4. The method of claim 3, wherein the change is decrease in the level of the gene transcript expression or protein expression from the first timepoint as compared to the second timepoint indicates in improved prognosis of RCC progression at the second timepoint as compared to the first timepoint.

5. The method of claim 3, wherein a change is an increase in the level of the gene transcript expression or protein expression from the first timepoint as compared to the second timepoint indicates in decreased prognosis of RCC progression at the second timepoint as compared to the first timepoint.

6. The method of claim 2, wherein the gene group comprises at least 3 sequences of genes selected from the group consisting of GenBank identification Nos. or Unigene identification Nos: NM_{—}001218///NM_{—}017689///AF051882 (SEQ ID NO:1); NM_{—}001216///X66839 (SEQ ID NO:2); NM_{—}022073///NM_{—}033344///AJ310545 (SEQ ID NO:3); NM_{—}013332 (SEQ ID NO:4); NM_{—}003239 (SEQ ID NO:5); NM_{—}006681///X76029 (SEQ ID NO:6); NM_{—}000304///D11428 (SEQ ID NO:7); NM_{—}007257///XM_{—}376764 (SEQ ID NO:8); M63928///NM_{—}001033126///XM_{—}284241 (SEQ ID NO:9); U19869///NM_{—}001040442///NM_{—}001445 (SEQ ID NO:10); NM_{—}000909 (SEQ ID NO:11) and NM_{—}001252///L08096 (SEQ ID NO:12).

7-17. (canceled)

18. The method of claim 2, wherein the biological sample is selected from the group of serum, blood, plasma, urine, a tissue sample, biopsy tissue sample, stool, spinal fluid, sputum, nipple aspirates, lymph fluid, external secretions of the skin, respiratory tract, intestinal and genitourinary tracts, bile, saliva, milk, tumors, organs and also samples of in vitro cell culture constituents.

19. (canceled)

20. (canceled)

21. The method of claim 2, wherein the protein expression is detected using an antibody, human antibody, humanized antibody, recombinant antibodies, monoclonal antibodies, chimeric antibodies, aptamer, peptide or analogues, or conjugates or fragments thereof.

22. The method of claim 21, wherein detection is by ELISA.

23. The method of claim 2, wherein the gene transcript expression is detected at the level of messenger RNA (mRNA).

24. The method of claim 23, wherein detection uses nucleic acid or nucleic acid analogues.

25. (canceled)

26. (canceled)

27. The method of claim 2, wherein a clinician directs the subject to be treated with an appropriate therapy if the subject has, or is at risk of developing RCC.

28. (canceled)

29. (canceled)

30. (canceled)

31. An array comprising a solid platform and attached the solid platform are protein-binding molecules, wherein the array comprises at most 100 different protein-binding molecules in known positions, wherein at least three of the 100 different protein-binding molecules have a specific binding affinity for proteins selected from the group of CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43) or fragments or functional variants or derivatives thereof.

32. The array of claim 31, wherein the array is used in the methods to identify a subject having increased likelihood of developing or having renal cell carcinoma (RCC) according to claim 2.

33.-42. (canceled)

43. The array of claim 31, wherein the array is an ELISA kit or a Multiplex Immunoassay.

44. The array of claim 31, wherein the array is a protein chip.

45. The array of claim 31, wherein the array comprises at the most 50 different protein binding molecules in known positions, wherein at least three of the 50 different protein-binding molecules have a specific binding affinity for proteins selected from the group of CA12 (SEQ ID NO: 32); CA9 (SEQ ID NO: 33); EGLN3 (SEQ ID NO: 34); HIG2 (SEQ ID NO: 35); TGFB3 (SEQ ID NO: 36); NMU (SEQ ID NO: 37); PMP22 (SEQ ID NO: 38); PNMA2 (SEQ ID NO: 39); TNFRSF7 (SEQ ID NO: 40); FABP6 (SEQ ID NO: 41); CD70 (CD27L) (SEQ ID NO: 42); NPY1 (SEQ ID NO: 43) or fragments or functional variants or derivatives thereof.

46. The method of claim 3, wherein the gene group comprises at least 3 sequences of genes selected from the group consisting of GenBank identification Nos. or Unigene identification Nos: NM_{—}001218///NM_{—}017689///AF051882 (SEQ ID NO:1); NM_{—}001216///X66839 (SEQ ID NO:2); NM_{—}022073///NM_{—}033344///AJ310545 (SEQ ID NO:3); NM_{—}013332 (SEQ ID NO:4); NM_{—}003239 (SEQ ID NO:5); NM_{—}006681///X76029 (SEQ ID NO:6); NM_{—}000304///D11428 (SEQ ID NO:7); NM_{—}007257///XM_{—}376764 (SEQ ID NO:8); M63928///NM_{—}001033126///XM_{—}284241 (SEQ ID NO:9); U19869///NM_{—}001040442///NM_{—}001445 (SEQ ID NO:10); NM_{—}000909 (SEQ ID NO:11) and NM_{—}001252///L08096 (SEQ ID NO:12).

47. The method of claim 3, wherein the biological sample is selected from the group of serum, blood, plasma, urine, a tissue sample, biopsy tissue sample, stool, spinal fluid, sputum, nipple aspirates, lymph fluid, external secretions of the skin, respiratory tract, intestinal and genitourinary tracts, bile, saliva, milk, tumors, organs and also samples of in vitro cell culture constituents.

48. The method of claim 3, wherein the protein expression is detected using an antibody, human antibody, humanized antibody, recombinant antibodies, monoclonal antibodies, chimeric antibodies, aptamer, peptide or analogues, or conjugates or fragments thereof.

49. The method of claim 48, wherein detection is by ELISA.

50. The method of claim 3, wherein the gene transcript expression is detected at the level of messenger RNA (mRNA).
